(19) United States
(12) Patent Application Publication
(10) Pub. No.: US 2021/0171437 A1
(43) Pub. Date: Jun. 10, 2021

(54) PEPTIDE NUCLEIC ACID (PNA) MONOMERS WITH AN ORTHOGONALLY PROTECTED ESTER MOIETY AND NOVEL INTERMEDIATES AND METHODS RELATED THERETO

(71) Applicant: VERA THERAPEUTICS, INC., South San Francisco, CA (US)

(72) Inventors: James M. Coull, Westford, MA (US); Brian D. Gildea, Bedford, MA (US)

(21) Appl. No.: 17/181,041

(22) Filed: Feb. 22, 2021

Related U.S. Application Data
(63) Continuation of application No. 16/037,953, filed on Jul. 17, 2018.
(60) Provisional application No. 62/634,680, filed on Feb. 23, 2018.
(60) Provisional application No. 62/533,582, filed on Jul. 17, 2017.

(51) Int. Cl. C07C 229/08; C07D 239/54; C07C 271/20; C07D 239/47; C07C 269/06; C07D 473/34; C07C 229/26; C07C 221/00; C07D 473/06; C07D 473/18; C07C 309/30; C07C 225/06; C07C 227/18; C07C 215/08; C07C 213/08

(52) U.S. Cl. CPC C07C 229/08; C07C 2603/18; C07C 271/20; C07D 239/47; C07C 269/06; C07D 473/34; C07C 229/26; C07C 221/00; C07D 473/06; C07D 473/18; C07C 309/30; C07C 225/06; C07C 227/18; C07C 215/08; C07C 213/08; C07D 239/54

(57) ABSTRACT

The present disclosure pertains to peptide nucleic acid (PNA) monomers and oligomers, as well as methods and compositions useful for the preparation of PNA monomer precursors (e.g. PNA Monomer Esters, Backbone Esters and Backbone Ester Acid Salts, as described below) that can be used to prepare PNA monomers wherein said PNA monomers can be used to prepare said PNA oligomers. In some embodiments, the disclosure features sulfonic acid salts of Backbone Ester compounds, which sulfonic acid salts generally tend to be crystalline and can be obtained in reasonably good yield, often without requiring any chromatographic purification of the reaction product of the Backbone Ester synthesis reaction. This disclosure also pertains to novel methods for the synthesis of said Backbone Ester compounds and novel methods for the formation of the related sulfonic acid salts. Exemplary ester groups include, but are not limited to, 2,2,2-trichloroethy-(TCE), 2,2,2-tribromoethyl-(TBE), 2-iodoethyl-groups (2-IE) and 2-bromoethyl-(2-BrE) as the ester group. These particular ester groups can be removed under conditions where both Boc and Fmoc protected amine groups are stable.

### CROSS REFERENCE TO RELATED APPLICATIONS

This application is a continuation of U.S. patent application Ser. No. 16/037,953, filed Jul. 17, 2018, which claims the benefit of U.S. Provisional Patent Application No. 62/533,582, filed on Jul. 17, 2017, and U.S. Provisional Patent Application No. 62/634,680, filed on Feb. 23, 2018. The disclosure of each of the foregoing applications is incorporated herein by reference in its entirety.

The section headings used herein are for organizational purposes only and should not be construed as limiting the subject matter described in any way.

### BRIEF DESCRIPTION OF DRAWINGS

The skilled artisan will understand that the drawings, described below, are for illustration purposes only. The drawings are not intended to limit the scope of the present teaching in any way. Drawings are not necessarily presented in any scale and should not be interpreted as implying any scale. In various figures and chemical formulas, a point of attachment to another moiety is sometimes illustrated for clarity.

FIG. 1 is an illustration of a classic peptide nucleic acid (PNA) monomer subunit (of a PNA oligomer) with its various subgroups identified.

FIG. 2 is an illustration of several common (but non-limiting) unprotected nucleobases (identified as ‘B’ in FIG. 1) that can be linked to a PNA monomer (or subunit of a polymer/oligomer). For those nucleobases with an exocyclic amine moiety, said exocyclic amine can be protected with a protecting group. In some embodiments, lactam and/or ring nitrogen groups of the nucleobase can be protected. In some embodiments, other groups or atoms (e.g. sulfur) of the nucleobase can optionally be protected.

FIG. 3 is an illustration of various exemplary nucleobases commonly used in PNA synthesis. For those nucleobases with an exocyclic amine moiety, said exocyclic amine can be protected with a protecting group. In some embodiments, lactam and/or ring nitrogen groups of the nucleobase can be protected. In some embodiments, other groups or atoms (e.g. sulfur) of the nucleobase can optionally be protected.

FIG. 4 is an illustration of several exemplary base-labile N-terminal amine protecting groups that can be used in an orthogonal protection scheme for the N-terminal amine group of PNA monomers or PNA Monomer Esters (e.g., as described herein) as contemplated by some embodiments of the present invention.

FIG. 5 an illustration of several exemplary acid-labile N-terminal amine protecting groups that can be used in an orthogonal protection scheme for the N-terminal amine group of PNA monomers or PNA Monomer Esters (e.g., as described herein) as contemplated by some embodiments of the present invention.

FIG. 6a is an illustration of several exemplary base-labile exocyclic amine protecting groups that can be used in an orthogonal protection scheme for the nucleobases of PNA monomers or PNA Monomer Esters (e.g., as described herein) as contemplated by some embodiments of the present invention.

FIG. 6b is an illustration of several exemplary acid-labile exocyclic amine protecting groups (or protecting group schemes such as Bis-Boc) that can be used in an orthogonal protection scheme for the nucleobases of PNA monomers or PNA Monomer Esters (e.g., as described herein) as contemplated by some embodiments of the present invention.

FIG. 6c is an illustration of several exemplary imide and lactam protecting groups that can be used in an orthogonal protection scheme for the nucleobases of PNA monomers or PNA Monomer Esters as contemplated by some embodiments of the present invention.

FIG. 7 is an illustration of several exemplary groups/moieties that can be present as a side chain linked to an α, and/or γ carbon of the backbone of PNA monomers or PNA Monomer Esters (e.g., as described herein) as contemplated by some embodiments of the present invention. Because they only comprise carbon and hydrogen, moieties IIIa, IIIb, IIIc, IIId, IIIe, IIIf, IIIg and IIIh are generally considered to be unreactive and therefore not typically in need of a protecting group. Because they comprise an amine function, moieties IIIi, IIIj, IIIk and IIIm can be protected with an amine protecting group in PNA monomers or PNA Monomer Esters as contemplated by some embodiments of the present invention (See for example: FIGS. 9a and 9b, below). Because they comprise a sulfur atom, moieties IIIn, IIIo, and IIIp can be protected with a sulfur protecting group in the PNA monomers or PNA Monomer Esters as contemplated by some embodiments of the present invention (See for example: FIGS. 13a and 13b, below). Because they comprise a hydroxyl group, moieties IIIq, IIIr and IIIs can be protected with a hydroxyl protecting group in PNA monomers or PNA Monomer Esters as contemplated by some embodiments of the present invention (See for example: FIGS. 16a, 16b, 17a and 17, below).

FIG. 8 is an illustration of several exemplary groups/moieties that can be present as a side chain linked to an α, and/or γ carbon of the backbone of a PNA monomers or PNA Monomer Esters as contemplated by some embodiments of the present invention. Because they comprise a carboxylic acid function, moieties IIIt and IIIu can be protected with a carboxylic acid protecting group in the PNA monomers or PNA Monomer Esters as contemplated by some embodiments of the present invention (See for example: FIGS. 10a and 10b, below). Because they comprise an amide function, moieties IIIy and IIIw can be protected with an amide protecting group in the PNA monomers or PNA Monomer Esters as contemplated by some embodiments of the present invention (See for example: FIG. 11, below). Similarly, groups IIIx, IIIy and IIIz may comprise a protecting group suitable for said arginine, tryptophan and histidine side chains in the PNA monomers or PNA Monomer Esters as contemplated by some embodiments of the present invention (See FIGS. 12a, 12b, 14a, 14b, 15a and 15b, respectively). Preferred embodiments of a miniPEG side chain in the PNA monomers or PNA Monomer Esters as contemplated by some embodiments of the present invention are also illustrated as formula IIIaa or as a side chain of formula IIIab (wherein R₁₆ and n are defined below).

FIG. 9a is an illustration of several exemplary acid-labile protecting groups that can be used, inter alia, to protect amine containing side chain moieties such as those of formulas: IIIi, IIIj, IIIk and IIIm.

FIG. 9b is an illustration of several exemplary base-labile protecting groups that can be used, inter alia, to protect amine containing side chain moieties such as those of formulas: IIIi, IIIj, IIIk and IIIm.

FIG. 10a is an illustration of several exemplary acid-labile protecting groups that can be used, inter alia, to protect carboxylic acid containing side chain moieties such as those of formulas: IIIt and IIIu.

FIG. 10b is an illustration of several exemplary base-labile protecting groups that can be used, inter alia, to protect carboxylic acid containing side chain moieties such as those of formulas: IIIt and IIIu.

FIG. 11 is an illustration of several exemplary acid-labile protecting groups that can be used, inter alia, to protect amide containing side chain groups such as those of formulas: IIIy and IIIw.

FIG. 12a is an illustration of several exemplary acid-labile protecting groups that can be used, inter alia, to protect guanidinium containing side chain moieties such as those of formula: IIIx.

FIG. 12b is an illustration of an exemplary base-labile protecting group that can be used, inter alia, to protect guanidinium containing side chain moieties such as those of formula: IIIx.

FIG. 13a is an illustration of several exemplary acid-labile protecting groups that can be used, inter alia, to protect thiol containing side chain moieties such as those of formula: IIIn.

FIG. 13b is an illustration of several exemplary base-labile protecting groups that can be used, inter alia, to protect thiol containing side chain moieties such as those of formula: IIIn.

FIG. 14a is an illustration of several exemplary acid-labile protecting groups that can be used, inter alia, to protect indole side chain moieties such as those of formula: IIIy.

FIG. 14b is an illustration of an exemplary other protecting group that can be used, inter alia, to protect indole side chain moieties such as those of formula: IIIy.

FIG. 15a is an illustration of several exemplary acid-labile protecting groups that can be used, inter alia, to protect imidazole side chain moieties such as those of formula: IIIz.

FIG. 15b is an illustration of several exemplary base-labile protecting groups that can be used, inter alia, to protect imidazole side chain moieties such as those of formula: IIIz.

FIG. 16a is an illustration of several exemplary acid-labile protecting groups that can be used, inter alia, to protect hydroxyl containing moieties such as those of formulas: IIIq and IIIr.

FIG. 16b is an illustration of several exemplary other protecting groups that can be used, inter alia, to protect hydroxyl containing moieties such as those of formulas: IIIq and IIIr.

FIG. 17a is an illustration of several exemplary acid-labile protecting groups that can be used, inter alia, to protect phenolic containing moieties such as those of formula: IIIs.

FIG. 17b is an illustration of several exemplary other protecting groups that can be used, inter alia, to protect phenolic containing moieties such as those of formula: IIIs.

FIG. 18a is an illustration of various examples of suitable nucleobases (in unprotected form) that can be used in some of the novel PNA Monomer Ester embodiments of the present invention.

FIG. 18b is an illustration of various examples of suitable protected forms of the nucleobases illustrated in FIG. 18a that can be used in some of the novel PNA Monomer Ester embodiments of the present invention.

FIG. 19 is an illustration of exemplary methods for the preparation of various Amino Acid Ester and Amino Acid Ester Acid Salt compositions used in some embodiments of the present invention. In the illustration PgX represents an amine protecting group, PgA represents an acid-labile amine protecting group (e.g. Boc) and PgB represents a base-labile amine protecting group (e.g. Fmoc). Groups R₅, R₆, R₁₁, R₁₂, R₁₃, R₁₄ and Y⁻ are defined below.

FIG. 20 is an illustration of several exemplary synthetic paths to aldehyde compositions useful in the preparation of novel Backbone Ester (e.g., as described herein) and Backbone Ester Acid Salt (e.g., as described herein) compositions as contemplated by some embodiments of the present invention. Groups Pg₁, R₂, R₃ and Ra are as defined below.

FIG. 21 is an illustration of one (of several) possible synthetic routes to novel Backbone Ester and Backbone Ester Acid Salt compositions as contemplated by some embodiments of the present invention. Groups Pg₁, R₂, R₃, R₄, R₅, R₆, R₁₁, R₁₂, R₁₃, R₁₄ and Y⁻ are defined below.

FIG. 22 is an illustration of some possible methods for converting Backbone Ester and Backbone Ester Acid Salt compositions into PNA Monomer Ester compositions as contemplated by some embodiments of the present invention. Groups Pg₁, R₂, R₃, R₄, R₅, R₆, R₉, R₁₀, R₁₁, R₁₂, R₁₃, R₁₄ and Y⁻ are defined below. B is a nucleobase.

FIG. 23 is an illustration of some possible (non-limiting) methods for converting PNA Monomer Ester compositions into PNA Monomer (e.g., as described herein) compositions as contemplated by some embodiments of the present invention.

FIG. 24a is an image of overlaid HPLC traces showing the conversion of an exemplary PNA Monomer Ester composition into a PNA Monomer composition under certain conditions (See: Example 12).

FIG. 24b is an image of overlaid HPLC traces showing the conversion of an exemplary PNA Monomer Ester composition into a PNA Monomer composition under certain conditions (See: Example 12).

FIG. 25 is an image of overlaid HPLC traces showing the conversion of an exemplary PNA Monomer Ester composition into a PNA Monomer composition under certain conditions (See: Example 13).

FIG. 26a is an image of overlaid HPLC traces showing the conversion of an exemplary PNA Monomer Ester composition into a PNA Monomer composition under certain conditions (See: Example 13).

FIG. 26b is an image of overlaid HPLC traces showing the conversion of an exemplary PNA Monomer Ester composition into a PNA Monomer composition under certain conditions (See: Example 13).

FIG. 27A is an illustration of a novel method for the production of Backbone Ester Acid Salt compositions.

FIG. 27B is an illustration of a novel method for the production of Backbone Ester Acid Salt compositions.

FIG. 27C is an illustration of a way to convert commercially available N-Boc-ethylene diamine to a derivative of ethylene diamine comprising base-labile protecting group such as Fmoc.

FIG. 28A is an illustration of several exemplary Backbone Ester Acid Salt compositions.

FIG. 28B is an illustration of several exemplary Backbone Ester Acid Salt compositions.

FIG. 28C is an illustration of several exemplary Backbone Ester Acid Salt compositions.

All literature and similar materials cited in this application, including but not limited to patents, patent applications, articles, books and treatises, regardless of the format of such literature or similar material, are expressly incorporated by reference herein in their entirety for any and all purposes.

### DESCRIPTION

### 1. Field

The present disclosure pertains to peptide nucleic acid (PNA) monomers and oligomers, as well as methods and compositions useful for the preparation of PNA monomer precursors (e.g. PNA Monomer Esters, Backbone Esters and Backbone Ester Acid Salts, as described below) that can be used to prepare PNA monomers wherein said PNA monomers can be used to prepare said PNA oligomers.

### 2. Introduction

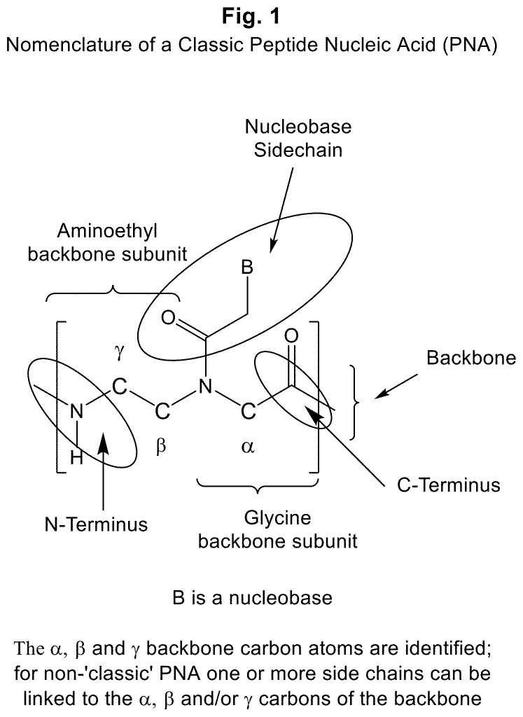

Peptide nucleic acid (PNA) oligomers are polymeric nucleic acid mimics that can bind to nucleic acids with high affinity and sequence specificity (See for example Ref A-1, B-1 and B-2). Despite its name, a peptide nucleic acid is neither a peptide, nor is it a nucleic acid. PNA is not a peptide because its monomer subunits are not traditional/natural amino acids or any amino acid that is found in nature (albeit natural amino acids and their side chains can, in some embodiments, be incorporated as subcomponent of a PNA monomer). PNA is not a nucleic acid because it is not composed of nucleoside or nucleotide subunits and is not acidic. A PNA oligomer is a polyamide. Accordingly, a PNA backbone typically comprises an amine terminus at one end and a carboxylic acid terminus at the other end (See: FIG. 1).

PNA oligomers are typically (but not exclusively) constructed by stepwise addition of PNA monomers. Each PNA monomer typically (but not necessarily) comprises both an N-terminal protecting group, a different/orthogonal protecting group for its nucleobase side chain that comprises an exocyclic amine (n.b. thymine and uracil derivatives usually don't require a protecting group) and a C-terminal carboxylic acid moiety. In some cases, other protecting groups are needed; for example, when a PNA monomer comprises an alpha, beta or gamma substituent having a nucleophilic, electrophilic or other reactive moiety (e.g. lysine, arginine, serine, aspartic acid or glutamic acid side chain moiety). See FIG. 1 for an illustration and nomenclature of the various subcomponents of a typical PNA subunit of a PNA oligomer.

Though not the only option, because PNA is a polyamide (as is a peptide), PNA oligomer synthesis has traditionally utilized much of the synthetic methodology and protecting group schemes developed for peptide chemistry, thereby facilitating its adaptation to automated instruments used for peptide synthesis. For example, the first commercially available PNA monomers were constructed using what is commonly referred to as Boc-benzyl (Boc/Cbz) chemistry (See for example Ref B-1 and B-2). More specifically, these PNA monomers (which were largely based on an aminoethylglycine backbone) utilized an N-terminal tert-butyloxycarbonyl (Boc or t-Boc group) to protect the terminal amine group and a benzyloxycarbonyl (Cbz or Z group) to protect the exocyclic amine groups of the adenine (A), cytosine (C) and guanine (G) nucleobases (i.e. thymine and uracil nucleobases typically do not comprise protecting groups). These PNA monomers are commonly referred to as ‘Boc/Z’ or ‘Boc/Cbz’ PNA monomers. While this protection scheme is workable (and typically produces products of superior purity as compared with Fmoc chemistry), because the boc group requires delivery of a strong acid such as trifluoroacetic acid (TFA) to the column at each synthetic cycle, this requirement makes this methodology less attractive to automation. It is noteworthy that the ‘Boc/Z’ or ‘Boc/Cbz’ PNA monomers are not truly orthogonal because both the Boc and Cbz groups are acid-labile, albeit true that the Cbz group requires significantly stronger acid for its removal as compared with the Boc protecting group.

To avoid the use of TFA, the base-labile Fluorenylmethoxycarbonyl (Fmoc) group is often used in peptide synthesis, including automated peptide synthesis. Today, most PNA oligomers are prepared from PNA monomers comprising the base-labile Fmoc group as the N-terminal amine protecting group of the PNA monomer. For the exocyclic amine groups of nucleobases, the acid-labile protecting groups benzhydroloxycarbonyl (Bhoc) and t-Boc (or Boc) have been used (See discussion in Example 11 and Table 11B, below). Accordingly, these PNA monomers are often referred to as Fmoc/Bhoc PNA monomers or Fmoc/t-Boc (or Fmoc/Boc) PNA monomers depending on the nature of the protecting group used on the exocyclic amine groups of the nucleobases.

Regardless of the nature of the N-terminal protecting group methodology employed, PNA monomers are most often prepared by saponification of a C-terminal methyl or ethyl ester with a strong base (such as sodium hydroxide or lithium hydroxide) followed by acidification to thereby produce a C-terminal carboxylic acid moiety (See for example Refs A-2, A-3 and B-3). For the Boc/Z protection methodology, this saponification process works well to thereby produce PNA monomers in high yield and high purity because neither the Boc group nor the Cbz group is base labile. However, if the PNA monomer precursor comprises a base-labile protecting group (e.g. Fmoc), this process generally leads to poor yields (typically less than 50% after column purification) of PNA monomer (especially as scale increases) that is often of inferior purity as compared with the Boc/Z PNA monomer counterparts.

Recently, the use of hydrogenation of PNA monomer benzyl esters has been employed to improve yield and purity (See: Ref C-27). As currently described, this process requires large quantities of solvent and there is a risk of hydrogenation of the double bond in the cytosine ring of the C-monomers.

The use of allyl esters has also been used as precursors in the preparation of PNA monomers (See: Ref C-36). As described, the allyl ester is removed by use of expensive palladium catalysts.

### 3. Definitions & Abbreviations

For the purposes of interpreting of this specification, the following definitions will apply and whenever appropriate, terms used in the singular will also include the plural and vice versa. In the event that any definition set forth below conflicts with the usage of that word in any other document, the definition set forth below shall always control for purposes of interpreting the scope and intent of this specification and its associated claims. Notwithstanding the foregoing, the scope and meaning of a word contained any document incorporated herein by reference should not be altered (for purposes of interpreting said document) by the definition presented below. Rather, said incorporated document (and words found therein) should be interpreted as it/they would be by the ordinary practitioner at the time of its publication based on its content and disclosure and when considered in terms of the context of the content of the description provided herein.

The use of “or” means “and/or” unless stated otherwise or where the use of “and/or” is clearly inappropriate. The use of “a” means “one or more” unless stated otherwise or where the use of “one or more” is clearly inappropriate. The use of “comprise,” “comprises,” “comprising”, “having”, “include,” “includes,” and “including” are interchangeable and not intended to be limiting.

Compounds described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

a. Abbreviations:

As used herein, the abbreviations for any protective groups, amino acids, reagents and other compounds are, unless clearly stated otherwise herein (e.g. in the Abbreviations Table below), in accord with their common usage, or the IUPAC-IUB Commission on Biochemical Nomenclature, Biochem., 11:942-944 (1972). The following abbreviations set forth in the Abbreviations Table supersede any other reference sources for purposes of interpreting this specification:

Abbreviations Table:

| Abbreviation | Compound Name |
|---|---|
| Ac | Acetyl |
| ACN | Acetonitrile |
| 1-Ada | 1-adamantyl |
| aeg | aminoethylglycine |
| Al | Allyl |
| Alloc | allyloxycarbonyl |
| Azoc | azidomethyloxycarbonyl |
| Bn | Benzyl |
| Boc or t-Boc | tert-butyloxycarbonyl |
| Bom | benzyloxymethyl |
| Bpoc | 2-(4-biphenyl) isopropoxycarbonyl |
| 2-BE | 2-bromoethyl |
| BrBn | 2-bromobenzyl |
| BrPhF | 9-(4-bromophenyl)-9-fluorenyl |
| BrZ | 2-bromobenzyloxycarbonyl |
| Bsmoc | 1,1-dioxobenzo[b]thiophene-2-ylmethyloxycarbonyl |
| Bum | tert-butoxymethyl |
| Cam | carbamoylmethyl |
| cHx | Cyclohexyl |
| 2-CE | 2-chloroethyl |
| Cl-Z | 2-chlorobenzyloxycarbonyl |
| Cys | Cysteine |
| D | Deuterium |
| Dab | diaminobutyric acid |
| Dap | diaminopropionic acid |
| Dcb | 2,6-dichlorobenzyl |
| DCC | N,N′-dicyclohexylcarbodiimide |
| DCM | Dichloromethane |
| DCU | N,N′-dicyclohexylurea |
| Dde | (1-(4,4-dimethyl-2,6-dioxocyclohex-1-ylidene)-3- |
| | ethyl) |
| Ddz | α,α-dimethyl-3,5-dimethoxybenyloxycarbonyl |
| dio-Fmoc | 2,7-diisooctyl-Fmoc |
| DIPEA or | N,N-diisopropylethylamine |
| DIEA | |
| Dma | 1,1-dimethylallyl |
| Dmab | 4-(N-[1-(4,4-dimethyl-2,6-dioxocyclohexylidene)-3- |
| | methylbutyl]amino)benzyl |
| DMAP | N,N-dimethyl-4-aminopyridine |
| Dmb | 2,4-dimethoxybenzyl |
| Dmcp | Dimethylcyclopropylmethyl |
| DME | 1,2-dimethoxyethane |
| DMF | N,N-dimethylformamide |
| Dmnb | 4,5-dimethoxy-2-nitrobenzyloxycarbonyl |
| DMSO | dimethylsulfoxide |
| dNBS | 2,4-dinitrobenzenesulfonyl |
| Dnp | 2,4-dinitrophenyl |
| Dnpe | 2-(2,4-dinitrophenyl)ethyl |
| Doc | 2,4-dimethylpent-3-yloxycarbonyl |
| Dts | dithiasuccinoyl |
| DTT | Dithiothreitol |
| EDC | 1-Ethyl-3-(3-dimethylaminopropyl)carbodiimide |
| Esc | Ethanesulfonylethoxycarbonyl |
| EtOAc | Ethyl acetate |
| Fm | 9-fluorenylmethyl |
| Fmoc | 9-fluorenylmethoxycarbonyl |
| Fmoc(2F) | 2-fluoro-Fmoc |
| Fmoc* | 2,7-di-tert-butyl-Fmoc |
| For | Formyl |
| HATU | 1-[Bis(dimethylamino) methylene]-1H-1,2,3-triazolo[4,5- |
| | b]pyridinium 3-oxide hexafluorophosphate |
| HBTU | 3-[Bis(dimethylamino) methyliumyl]-3H-benzotriazol-1- |
| | oxide hexafluorophosphate |
| Hmb | 2-hydroxy-4-ethoxybenzyl |
| Hoc | cyclohexyloxycarbonyl |
| HOBt | 1-hydroxybenzotriazole |
| 2-IE | 2-iodoethyl |
| ivDde | 1-(4,4-dimethyl-2,6-dioxocyclohex-1-ylidene)-3- |
| | methylbutyl |
| Mbh | 4,4′-dimethoxybenzhydryl |
| Meb | p-methylbenzyl |
| Men | β-menthyl |
| MeSub | 2-methoxy-5-dibenzosuberyl |
| Met | Methionine |
| MIM | 1-methyl-3-indolylmethyl |
| Mio-Fmoc | 2-monoisooctyl-Fmoc |
| MIS | 1,2-dimethylindole-3-sulfonyl |
| Mmt | monomethoxytrityl |
| Mob | p-methoxybenzyl |
| Mpe | β-3-methylpent-3-yl |
| Msc | 2-(methylsulfonyl) ethoxycarbonyl |
| Mtr | 4-methoxy-2,3,6-trimethylphenylsulfonyl |
| Mts | mesitylene-2-sulfonyl |
| Mtt | 4-methyltrityl |
| NMM | N-methylmorpholine |
| NMP | N-methylpyrrolidone |
| NPPOC | 2-(2-nitrophenyl) propyloxycarbonyl |
| Nps | 2-nitrophenylsulfanyl |
| Npys | 3-nitro-2-pyridinesulfenyl |
| Nsc | 2-(4-nitropheylsulfonyl)ethoxycarbonyl |
| α-Nsmoc | 1,1-dioxonaphtho[1,2-b]thiophene |
| NVOC | 6-nitroveratryloxycarbonyl |
| oNBS | o-nitrobenzenesulfonyl |
| oNZ | o-nitrobenzyloxycarbonyl |
| Orn | ornithine |
| Pac | phenacyl |
| Pbf | pentamethyl-2,3-dihydrobenzofuran-5-sulfonyl |
| PhAcm | phenylacetamidomethyl |
| Phdec | phenyldithioethyloxycarbonyl |
| 2-PhⁱPr | 2-phenylisopropyl |
| pHP | p-hydroxyphenacyl |
| Pmbf | 2,2,4,6,7-pentamethyl-5-dihydrobenzofuranyl-methyl |
| Pmc | 2,2,5,7,8-pentamethylchroman-6-sulfonyl |
| Pms | 2-[phenyl(methyl)sulfonio]ethyloxycarbonyl |
| | tetrafluoroborate |
| pNB | p-nitrobenzyl |
| pNBS | p-nitrobenzenesulfonyl |
| pNZ | p-nitrobenzyloxycarbonyl |
| Poc | propargyloxycarbonyl |
| Pydec | 2-pyridyldithioethyloxycarbonyl |
| Sps | 2-(4-sulfophenylsulfonyl)ethoxycarbonyl |
| S-Pyr | 2-pyridinesulfenyl |
| S^{t}Bu | tert-butylmercapto |
| Sub | 5-dibenzosuberyl |
| Suben | ω-5-dibenzosuberenyl |
| TBDMS | tert-butyldimethylsilyl |
| TBDPS | tert-butyldiphenylsily |
| ^{t}Bu | tert-butyl |
| Tbe | 2,2,2-tribromoethyl |
| TBP | Tri-n-butyl-phosphine |
| TCE | 2,2,2-trichloroethyl |
| TCP | tetrachlorophthaloyl |
| TEA | trimethylamine |
| TFA | trifluoroacetic acid |
| TFMSA | trifluoromethanesulfonic acid |
| THF | tetrahydrofuran |
| TMAC | trimethylacetyl chloride |
| Tmob | 2,4,6-trimethoxybenzyl |
| TMSE | trimethylsilylethyl |
| Tmsi | 2-(trimethylsilyl)isopropyl |
| Ts | Tosyl (a.k.a. p-toluenesulfonyl) |
| Troc | 2,2,2-trichloroethyloxycarbonyl |
| Trp | tryptophan |
| Trt | trityl |
| Xan | 9-xanthenyl |
| Z or cbz/Cbz | benzyloxycarbonyl |

b. Technology Specific Definitions

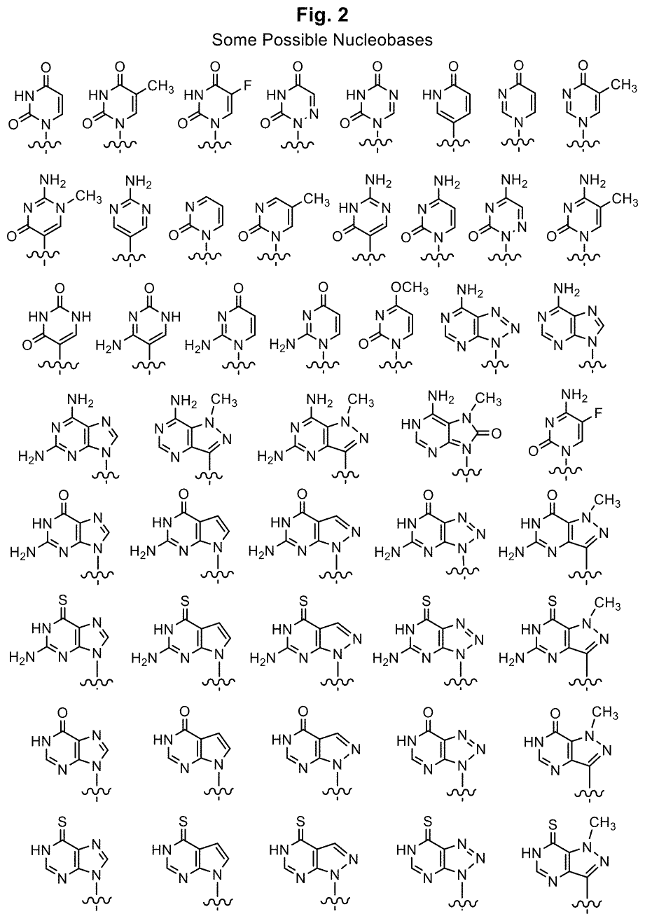

As used herein, the term “nucleobase” means those naturally occurring and those non-naturally occurring cyclic moieties used to thereby generate polymers that sequence specifically hybridize/bind to nucleic acids by any means, including without limitation through Watson-Crick and/or Hoogsteen binding motifs. Some non-limiting examples of nucleobases can be found in FIGS. 2, 3, 6c, 18a and 18b.

As used herein, the term “orthogonal protection” refers a strategy of allowing the deprotection of multiple protective groups one at a time each with a dedicated set of reaction conditions without affecting the other protecting groups or the functional groups protected thereby.

As used herein, the term “pharmaceutically acceptable salt” refers to salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention. In some embodiments, a pharmaceutically acceptable salt is a benzenesulfonic acid salt, a p-tosylsulfonic acid salt, or a methanesulfonic acid salt.

As used herein, the term “protecting group” refers to a chemical group that is reacted with, and bound to (at least for some period of time), a functional group in a molecule to prevent said functional group from participating in reactions of the molecule but which chemical group can subsequently be removed to thereby regenerate said functional group. Additional reference is made to: Oxford Dictionary of Biochemistry and Molecular Biology, Oxford University Press, Oxford, 1997 as evidence that protecting group is a term well-established in field of organic chemistry.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like.

As used herein, the term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0.

As used herein, the term “tautomer” as used herein refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

c. Chemical Definitions:

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 8 carbon atoms (“C₁-C₈ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is substituted C₁₋₆ alkyl.

As used herein, “alkenyl” refers to a radical of a straight-chain or branched hydrocarbon group having from 2 to 10 carbon atoms, one or more carbon-carbon double bonds, and no triple bonds (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 to 5 carbon atoms (“C₂-C₅ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₆ alkenyl.

As used herein, the term “alkynyl” refers to a radical of a straight-chain or branched hydrocarbon group having from 2 to 10 carbon atoms, one or more carbon-carbon triple bonds (“C₂-C₂₄ alkenyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 to 5 carbon atoms (“C₂-C₅ alkynyl”). In some embodiments, an alkynyl group has 2 to 4 carbon atoms (“C₂-C₄ alkynyl”). In some embodiments, an alkynyl group has 2 to 3 carbon atoms (“C₂-C₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, the terms “arylene” and “heteroarylene,” alone or as part of another substituent, mean a divalent radical derived from an aryl and heteroaryl, respectively. Each instance of an arylene or heteroarylene may be independently optionally substituted, i.e., unsubstituted (an “unsubstituted arylene”) or substituted (a “substituted heteroarylene”) with one or more substituents.

As used herein, the term “arylalkyl” refers to an aryl or heteroaryl group that is attached to another moiety via an alkylene linker. As used herein, the term “arylalkyl” refers to a group that may be substituted or unsubstituted. The term “arylalkyl” is also intended to refer to those compounds wherein one or more methylene groups in the alkyl chain of the arylalkyl group can be replaced by a heteroatom such as —O—, —Si— or —S—.

As used herein, “cycloalkyl” refers to a radical of a non-aromatic cyclic hydrocarbon group having from 3 to 7 ring carbon atoms (“C₃-C₇ cycloalkyl”) and zero heteroatoms in the non-aromatic ring system. In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 7 ring carbon atoms (“C₅-C₇ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₇ cycloalkyl groups include, without limitation, the aforementioned C₃-C₆ cycloalkyl groups as well as cycloheptyl (C₇), cycloheptenyl (C₇), cycloheptadienyl (C₇), and cycloheptatrienyl (C₇), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

As used herein, the term “heteroalkyl” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CHs, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. The term “alkenylene,” by itself or as part of another substituent, means, unless otherwise stated, a divalent radical derived from an alkene. An alkylene, alkenylene, alkynylene, or heteroalkylene group may be described as, e.g., a C₁-C₆-membered alkylene, C₁-C₆-membered alkenylene, C₁-C₆-membered alkynylene, or C₁-C₆-membered heteroalkylene, wherein the term “membered” refers to the non-hydrogen atoms within the moiety. In the case of heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—. Each instance of an alkylene, alkenylene, alkynylene, or heteroalkylene group may be independently optionally substituted, i.e., unsubstituted (an “unsubstituted alkylene”) or substituted (a “substituted heteroalkylene) with one or more substituents.

As used herein, the term “heteroaryl,” refers to an aromatic heterocycle that comprises 1, 2, 3 or 4 heteroatoms selected, independently of the others, from nitrogen, sulfur and oxygen. As used herein, the term “heteroaryl” refers to a group that may be substituted or unsubstituted. A heteroaryl may be fused to one or two rings, such as a cycloalkyl, an aryl, or a heteroaryl ring. The point of attachment of a heteroaryl to a molecule may be on the heteroaryl, cycloalkyl, heterocycloalkyl or aryl ring, and the heteroaryl group may be attached through carbon or a heteroatom. Examples of heteroaryl groups include imidazolyl, furyl, pyrrolyl, thienyl, thiazolyl, isoxazolyl, isothiazolyl, thiadiazolyl, oxadiazolyl, pyridinyl, pyrimidyl, pyrazinyl, pyridazinyl, quinolyl, isoquinolinyl, indazolyl, benzoxazolyl, benzisooxazolyl, benzofuryl, benzothiazolyl, indolizinyl, imidazopyridinyl, pyrazolyl, triazolyl, oxazolyl, tetrazolyl, benzimidazolyl, benzoisothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidyl, pyrazolo[3,4]pyrimidyl or benzo(b)thienyl, each of which can be optionally substituted.

As used herein, the term “heterocyclic ring” refers to any cyclic molecular structure comprising atoms of at least two different elements in the ring or rings. Additional reference is made to: Oxford Dictionary of Biochemistry and Molecular Biology, Oxford University Press, Oxford, 1997 as evidence that heterocyclic ring is a term well-established in field of organic chemistry.

d. Stereochemistry Considerations

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. In some embodiments, ‘substantially free’, refers to: (i) an aliquot of an “R” form compound that contains less than 2% “S” form; or (ii) an aliquot of an “S” form compound that contains less than 2% “R” form. The term “enantiomerically pure” or “pure enantiomer” denotes that the compound comprises more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure “R” form compound can comprise, for example, about 90% excipient and about 10% enantiomerically pure “R” form compound. In certain embodiments, the enantiomerically pure “R” form compound in such compositions can, for example, comprise, at least about 95% by weight “R” form compound and at most about 5% by weight “S” form compound, by total weight of the compound. For example, a pharmaceutical composition comprising enantiomerically pure “S” form compound can comprise, for example, about 90% excipient and about 10% enantiomerically pure “S” form compound. In certain embodiments, the enantiomerically pure “S” form compound in such compositions can, for example, comprise, at least about 95% by weight “S” form compound and at most about 5% by weight “R” form compound, by total weight of the compound. In certain embodiments, the active ingredient can be formulated with little or no excipient or carrier.

### 4. General

Herein described are alternative methods and compositions that can be used to produce PNA Monomer Esters that can, in a process that is amenable to scaling, yield PNA monomers (as free carboxylic acids) in high yield and high purity without regard to the presence of a base-labile protecting group such as Fmoc.

I. Nomenclature of a PNA Monomer, PNA Subunits, & PNA Oligomers

With reference to FIG. 1, a single subunit of a ‘classic’ PNA oligomer is illustrated within the bracketed region. By ‘classic’ we mean a PNA comprising an unsubstituted aminoethylglycine backbone (i.e. the —N—C—C—N—C—C(═O)—), wherein the aminoethyl subunit/group and the glycine subunit/group are called out and the α, β and γ carbon atoms of this aminoethylglycine backbone are identified. Because PNA is a polyamide, each subunit (and the oligomer also) comprises an amine terminus (i.e. N-terminus) and a carboxyl terminus (i.e. C-terminus). Each PNA subunit also comprises a nucleobase side chain, wherein the nucleobase (referred to in the illustration as B) is often (but not exclusively) attached to the backbone through a methylene carbonyl linker (as depicted).

Though a ‘classic’ PNA subunit is illustrated in FIG. 1, PNA subunits can comprise linked moieties at their α, β and/or γ carbon atoms and these linked moieties are also called side chains (or substituents) or more specifically, an α-sidechain (or α-substituent), a β-sidechain (or β-substituent) or a γ-sidechain (or γ-substituent). When substituted at its α, β or γ carbon atoms, the PNA subunit (or oligomer) is no longer referred to as ‘classic’.

As used herein, a PNA oligomer is any polymeric composition of matter comprising two or more PNA subunits of formula XV:

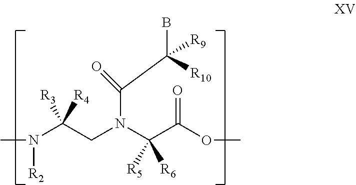

wherein, B, R₂, R₃, R₄, R₅, R₆, R₉ and R₁₀ are as defined anywhere herein and the points of attachment of the subunit within the polymer are as illustrated. In some embodiments, the PNA subunits are directly linked to one more other PNA subunits. In some embodiments, the two or more PNA subunits are not directly linked to another PNA subunit.

II. Backbone

Because of the availability of naturally occurring L-amino acids (and the counterpart non-naturally occurring D-amino acids and some of the methodologies available for producing the PNA backbone (as illustrated herein and demonstrated in the Examples below), substitution at the α-carbon and the γ-carbon of a PNA backbone with one or more amino acid side chain moieties can be readily accomplished by judicious selection of the input starting materials. Thus, myriad modifications of the ‘classic’ PNA subunit/backbone are possible.

Though many side-chain modifications (i.e. moieties linked at the α, β and/or γ carbon atoms of the aminoethylglycine unit) are possible without significantly inhibiting hybridization properties, alteration of the basic six atoms along of the PNA backbone (i.e. the carbon and nitrogen atoms making up the aminoethylglycine unit (i.e. —N—C—C—N—C—C(═O)—) generally has been shown to destroy (or substantially lower) hybridization potential of the resulting oligomer. Thus, aminoethylglycine unit (i.e. —N—C—C—N—C—C(═O)—, whether substituted or unsubstituted) is a feature of the most commonly used/described PNA oligomers. Furthermore, like the repeating sugar-phosphodiester backbone of a DNA or RNA, the repeating aminoethylglycine backbone of a PNA is the scaffold to which the nucleobases are linked in a way that provides for the just the right spacing, flexibility and orientation to permit sequence specific Watson-Crick and Hoogsteen binding/hybridization of these polymers to other PNA oligomers and to complementary DNA and RNA molecules.

III. Nucleobases

As noted above, a nucleobase is commonly attached to the backbone of each PNA subunit, typically via a methylene carbonyl linkage (See: FIG. 1). Nucleobases that can be attached to a PNA are generally not limited in any particular way except by their availability or by their inherent properties for binding to their complementary nucleobase in a binding motif. As is well known, nucleobases are generally either purines or pyrimidines, wherein (in Watson-Crick binding) the purines bind to complementary pyrimidines by hydrogen bonding (and base stacking) interactions.

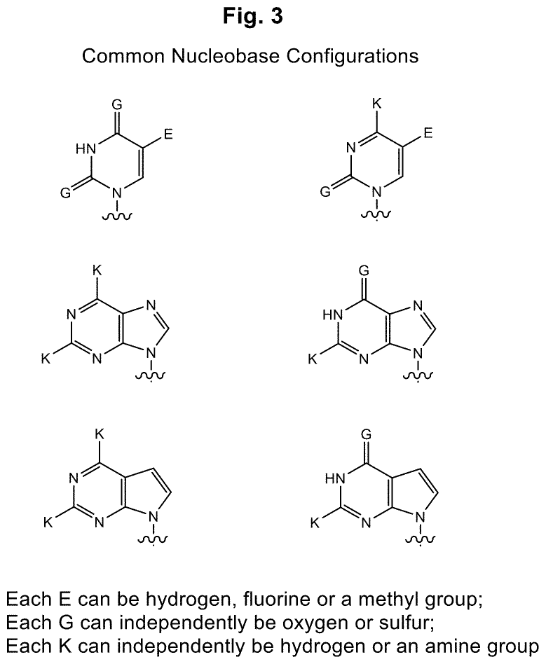

There are many modified nucleobases that have been developed over time and tested for function or unique binding or other properties in nucleic acid chemistry. These modified nucleobases are equally interesting as candidates for experimentation in PNA oligomers. Consequently, FIG. 2 provides an illustration of numerous nucleobases that can be incorporated into a PNA monomer to thereby produce a PNA subunit comprising said nucleobase, wherein the point of attachment to the PNA subunit is depicted. Some of the more common nucleobases are illustrated in FIG. 3, wherein the point of attachment to the PNA subunit is depicted. Methodologies for producing the nucleobase acids (e.g., nucleobase acetic acids) that can be linked to the backbone (for example, as described herein in Example 10) are well known (See for example: Refs: A-1, A-2, A-3, A-4, B-1, B-2 and C-27). All these embodiments of nucleobases (and any others that can be used in nucleic acid chemistry) are considered as useful for (and within the scope of all) embodiments of the present invention. In some embodiments, the nucleobases used can comprise one or more protecting groups.

A non-limiting list of nucleobases includes: adenine, guanine, thymine, cytosine, uracil, pseudoisocytosine, 2-thiopseudoisocytosine, 5-methylcytosine, 5-hydroxymethyl cytosine, xanthine, hypoxanthine, 2-aminoadenine (a.k.a. 2,6-diaminopurine), 2-thiouracil, 2-thiothymine, 2-thiocytosine, 5-chlorouracil, 5-bromouracil, 5-iodouracil, 5-chlorocytosine, 5-bromocytosine, 5-iodocytosine, 5-propynyl uracil, 5-propynyl cytosine, 6-azo uracil, 6-azo cytosine, 6-azo thymine, 7-methylguanine, 7-methyladenine, 8-azaguanine, 8-azaadenine, 7-deazaguanine, 7-deazaadenine, 3-deazaguanine, 3-deazaadenine, 7-deaza-8-aza guanine, 7-deaza-8-aza adenine, 5-propynyl uracil and 2-thio-5-propynyl uracil, including tautomeric forms of any of the foregoing.

IV. PNA Monomers and PNA Oligomer Synthesis

PNA oligomers are often prepared by stepwise addition of PNA monomers to form a growing polyamide chain, or by coupling smaller fragments of PNA together to generate the desired PNA oligomer. Synthesis of a PNA oligomer may make use of solid phase or solution phase techniques. In some embodiments, a PNA oligomer is prepared on a solid support, in which the first step entails linking a first PNA monomer to a resin bound linker. Synthesis is usually performed on a solid support using an automated instrument that delivers reagents to the support in a stepwise (and/or serial) fashion, but synthesis can be carried out in solution if so desired. In short, PNA synthesis generally mirrors peptide synthesis albeit with PNA monomers used as a substitute for the standard amino acid monomers. In this method, each PNA monomer adds a PNA subunit to the growing polyamide. Because PNA is a polyamide (like a peptide), many of the protecting group schemes, methodologies, resins, coupling agents, linkers and protecting groups have been adopted from standard peptide synthesis regimens. Thus, a PNA monomer generally mimics a protected amino acid suitable for use in peptide synthesis. In fact, because of the similarities, PNA monomers and protected amino acids are often used in the same protocols to produce hybrid oligomers that comprise both PNA subunits and amino acid subunits. For a more in-depth review of PNA synthesis methodologies and protection schemes, please see: Peptide Nucleic Acids, Protocols and Applications, Second Edition, Edited by Peter E. Nielsen, Horizon Bioscience, 2004 (ISBN 0-9545232-5), incorporated herein by reference.

V. N-terminal Protecting Groups

The N-terminus of a PNA monomer generally comprises an appropriate amine protecting group. In standard PNA synthesis (as in peptide synthesis), this group protects the terminal amine (i.e. in PNA synthesis—the nitrogen in bold underline of the aminoethylglycine unit (—N—C—C—N—C—C(═O)—) from reaction during coupling of the PNA monomer to the growing polyamide (or to the support, as the case may be); wherein said coupling is effected by amide bond formation through reaction of a resin bound amine group with the carboxylic acid function of the PNA monomer.

By judicious choice of protecting groups for the amino acid monomers, peptide synthesis has been shown to proceed by use of both acid-labile and base-labile protecting groups for the N-terminal amine (See: Ref: C-11 entitled: Amino Acid-Protecting Groups, and references cited therein; which reference provides a comprehensive review of protecting groups used in amino acid synthesis). By analogy, the use of both acid-labile protection of the N-terminal amine (See: Refs A-4, A-4, B-1, B-2, B-4) and base-labile protection (See: Refs A-2, A-5, B-3 and B-5) of the N-terminal amine of PNA monomers has been successfully used in PNA oligomer synthesis.

Therefore, as used herein, the abbreviation Pg₁ or PgX is used to denote an N-terminal amine protecting group that can be acid-labile or that can be base-labile. When intended to signify that the N-terminal amine protecting group is acid-labile, the abbreviation, PgA is used. When intended to signify that the N-terminal amine protecting group is base-labile, the abbreviation, PgB is used.

Non-limiting examples of suitable base-labile N-terminal amine protecting groups (i.e. PgB) that can be used in PNA monomers according to embodiments of this invention include: Fmoc, Nsc, Bsmoc, Nsmoc, ivDde, Fmoc*, Fmoc(2F), mio-Fmoc, dio-Fmoc, TCP, Pms, Esc, Sps and Cyoc. These base-labile protecting groups are illustrated in FIG. 4 and can be removed under conditions described in Ref. A-4 and Ref. C-11 and references cited therein.

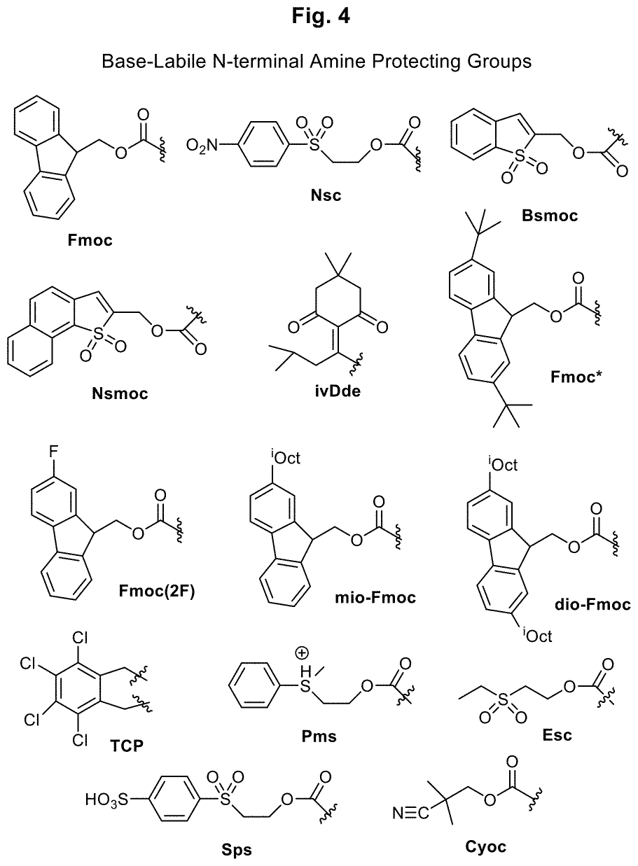

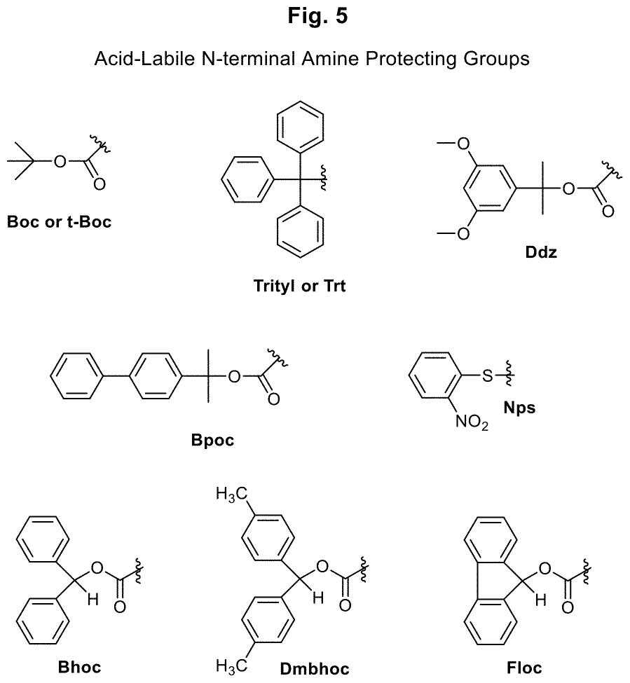

Non-limiting examples of suitable acid-labile N-terminal amine protecting groups (i.e. PgA) that can be used in PNA monomers according to embodiments of this invention include: Boc (or Boc), Trt, Ddz, Bpoc, Nps, Bhoc, Dmbhoc and Floc. These groups are illustrated in FIG. 5 and can be removed under conditions described in Ref. A-4 and Ref. C-11 and references cited therein.

VI. Nucleobases and Nucleobase Protecting Groups

As in chemical DNA synthesis, certain of the functional groups of nucleobases (of the PNA monomers and growing PNA oligomers) are best protected during PNA synthesis. However, there are reports of performing PNA synthesis without nucleobase protection (See for example: Ref. B-5) and such embodiments are also within the scope of the present invention. For this reason, the nucleobases are said to ‘optionally comprise one or more protecting groups’. Because of the long and well-developed history of nucleic acid synthesis chemistry, there are numerous existing nucleobase protecting groups that exist in the chemical literature. Generally, these are compatible with PNA synthesis. For a list of various known nucleobase protecting groups known in the nucleic acid field, please see Ref. C-13, and references cited therein. Various other nucleobase protecting groups that have been used in PNA synthesis can be found in Refs. A-1 to A-5 and B-1 to B-5).

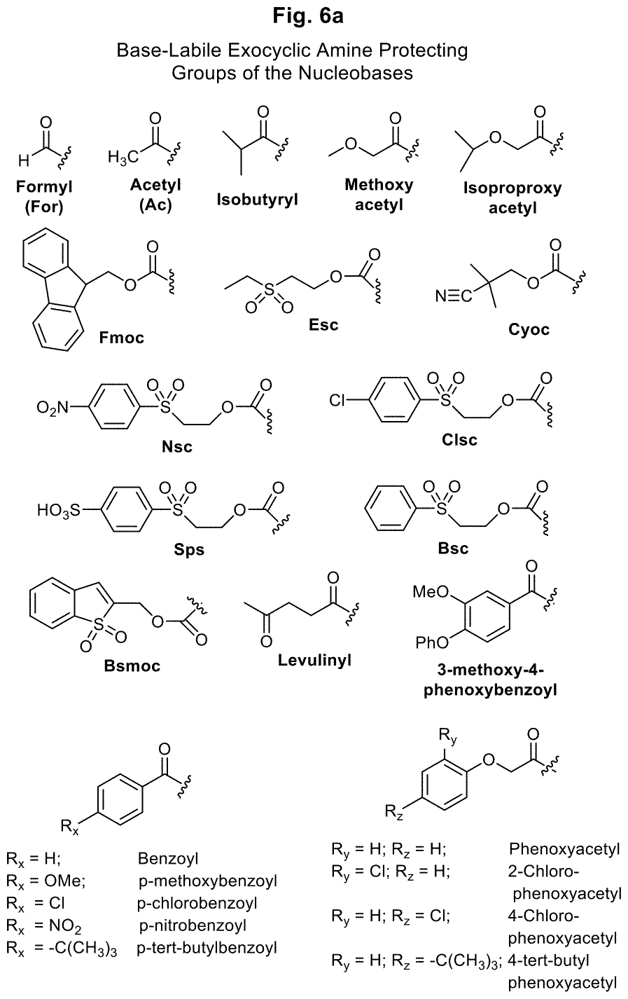

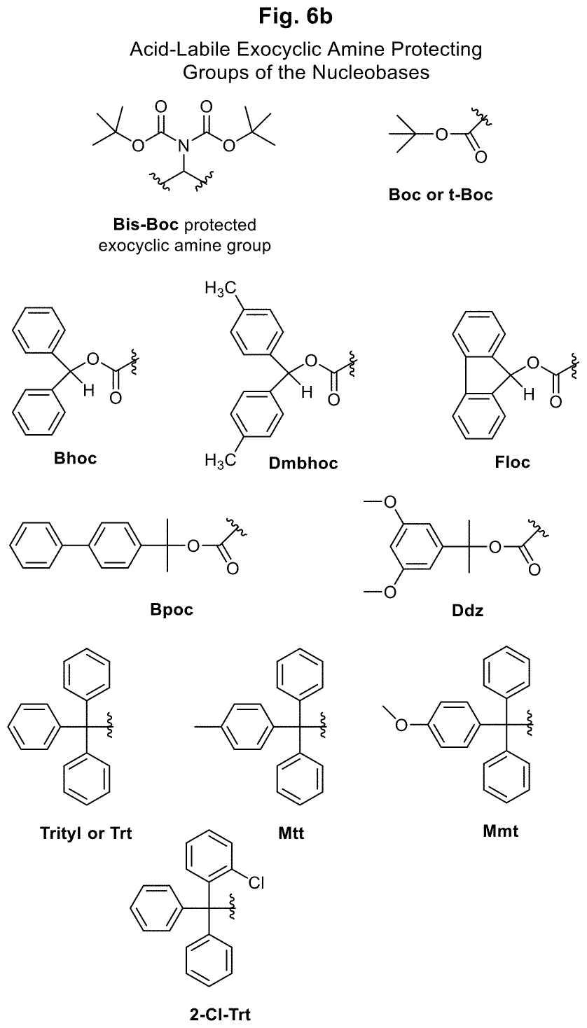

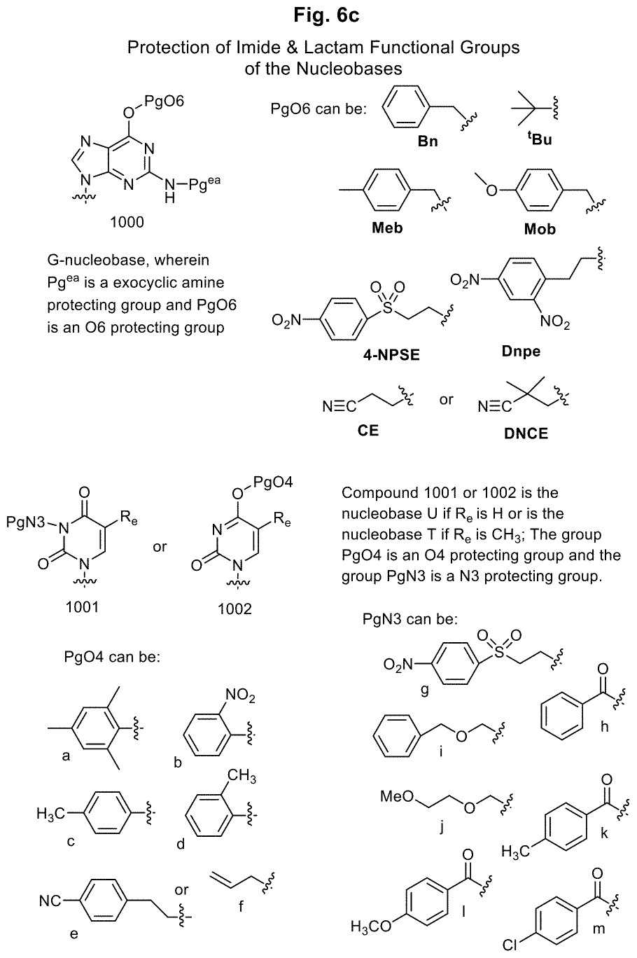

For example, if the N-terminal amine protecting group (which is typically removed at every synthetic cycle) is acid-labile (i.e. denoted PgA), then any nucleobase protecting groups are generally selected to be base-labile or removed under conditions of neutral pH. In short, the protecting groups for the N-terminal amine and the protecting groups for the nucleobases should likely be orthogonal. For example, the exocyclic amine groups of nucleobases are typically protected during PNA synthesis so that no unwanted coupling of PNA monomers occurs by reaction with these amine groups. With reference to FIG. 6a, numerous base-labile protecting groups are illustrated and can be used to protect the exocyclic amine groups of PNA monomers, and synthetic intermediates thereto, that can be used in embodiments of this invention. These include (but are not limited to), formyl, acetyl, isobutyryl, methoxyacetyl, isopropoxyacetyl, Fmoc, Esc, Cyoc, Nsc, Clsc, Sps, Bsc, Bsmoc, Levulinyl, 3-methoxy-4-phenoxybenzoyl, benzoyl (and various other benzoyl derivatives) and phenoxyacetyl (and various other phenoxyacetyl derivatives). Other examples of nucleobase protecting groups can be found in Ref C-13.

Similarly, if the N-terminal amine protecting group is base-labile (i.e. denoted PgB), then any nucleobase protecting groups are generally selected to be acid-labile or removed under conditions of neutral pH. With reference to FIG. 6b, numerous acid-labile protecting groups are illustrated and can be used to protect the exocyclic amine groups of PNA monomers, and synthetic intermediates thereto, that are used embodiments of this invention. These include (but are not limited to), Boc (sometimes abbreviated Boc or t-Boc), Bis-Boc (which means two Boc groups linked to the same amine group—as illustrated in FIG. 6b), Bhoc, Dmbhoc, Floc, Bpoc, Ddz, Trt, Mtt, Mmt and 2-Cl-Trt.

Certain nucleobases, such as thymine and uracil often do not comprise a protecting group for PNA synthesis. However, the imide/lactam functional groups of pyrimidine nucleobases can be protected in some embodiments. Similarly, although the O-6 of the guanine is typically not protected, it can be protected in some embodiments. Some non-limiting examples of protecting groups that can be used in embodiments of this invention to protect the N3/O4 of a pyrimidine nucleobase (exemplary compounds 1001 or 1002 are illustrated) or the O6 of a purine nucleobase (exemplary compound 1000 is illustrated) can be found in FIG. 6c.

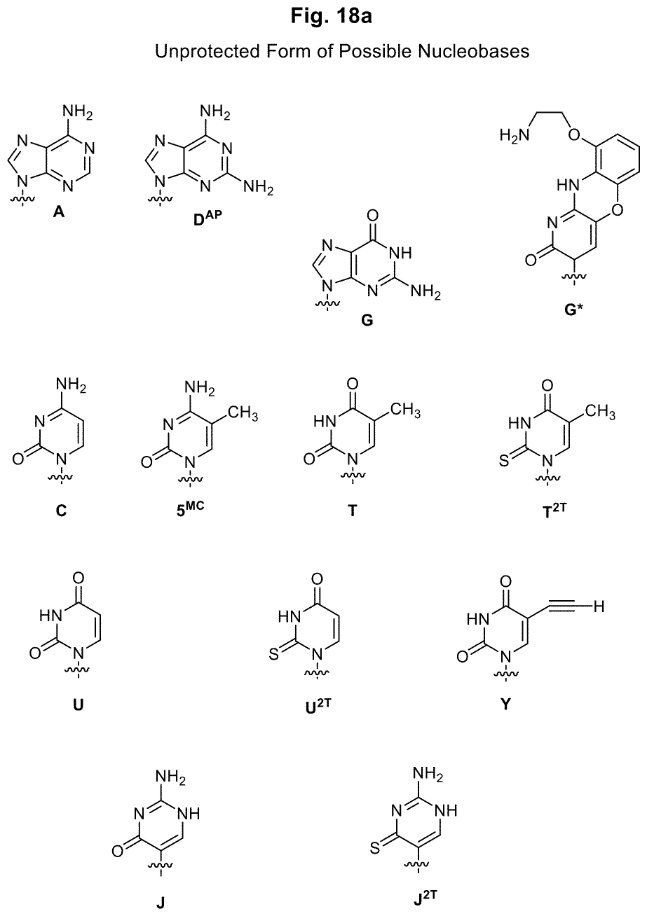

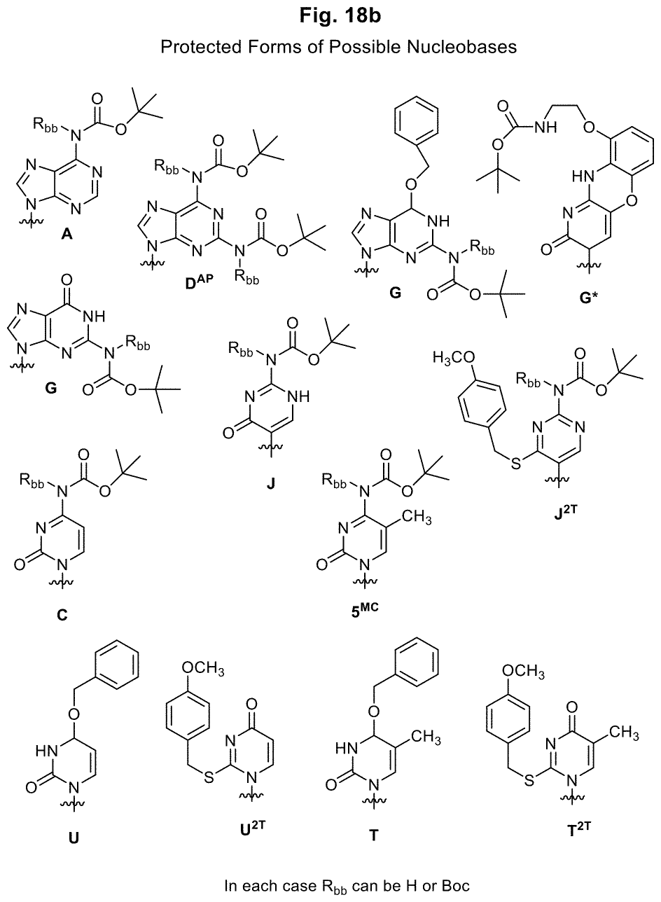

In addition to those nucleobases illustrated in FIGS. 2, 3, and 6c, FIG. 18a illustrates several common nucleobases herein identified as: A, D^{AP}, G, G*, C, 5^{MC}, T, T^{2T}, U, U^{2T}, Y, J and J^{2T} in unprotected form. FIG. 18b illustrates these nucleobases A, D^{AP}, G, G*, C, 5^{MC}, T, T^{2T}, U, U^{2T}, Y, J and J^{2T} as can be protected with an acid-labile protecting group for PNA synthesis (used for example where Pg₁ is selected to be base-labile).

A non-limiting list of nucleobases includes: adenine, guanine, thymine, cytosine, uracil, pseudoisocytosine, 2-thiopseudoisocytosine, 5-methylcytosine, 5-hydroxymethyl cytosine, xanthine, hypoxanthine, 2-aminoadenine (a.k.a. 2,6-diaminopurine), 2-thiouracil, 2-thiothymine, 2-thiocytosine, 5-chlorouracil, 5-bromouracil, 5-iodouracil, 5-chlorocytosine, 5-bromocytosine, 5-iodocytosine, 5-propynyl uracil, 5-propynyl cytosine, 6-azo uracil, 6-azo cytosine, 6-azo thymine, 7-methylguanine, 7-methyladenine, 8-azaguanine, 8-azaadenine, 7-deazaguanine, 7-deazaadenine, 3-deazaguanine, 3-deazaadenine, 7-deaza-8-aza guanine, 7-deaza-8-aza adenine, 5-propynyl uracil and 2-thio-5-propynyl uracil, including tautomeric forms of any of the foregoing.

VII. Amino Acid Side Chains and Their Protecting Groups

As described in more detail herein, in some embodiments of this invention, Backbone Ester compositions, Backbone Ester Acid Salt compositions and PNA Monomer Ester compositions can comprise one or more α- or γ-substituents (i.e. side chains). In some embodiments, these α- or γ-substituents are derived from (or have the chemical composition of) the side chains of naturally or non-naturally occurring amino acids.

For example and with reference to FIG. 7, in some embodiments, the α- or γ-substituents can be compositions of formula: IIIa (e.g., derived from alanine), IIIb (e.g., derived from aminobutyric acid), IIIc (e.g., derived from valine), IIId (e.g., derived from leucine), IIIe (e.g., derived from isoleucine), IIIf (e.g., derived from norvaline), IIIg (e.g., derived from phenylalanine) and/or IIIh (e.g., derived from norleucine). These α- or γ-substituents are all alkanes and therefore generally considered unreactive under conditions used in PNA synthesis. Accordingly, they typically do not comprise any protecting group.

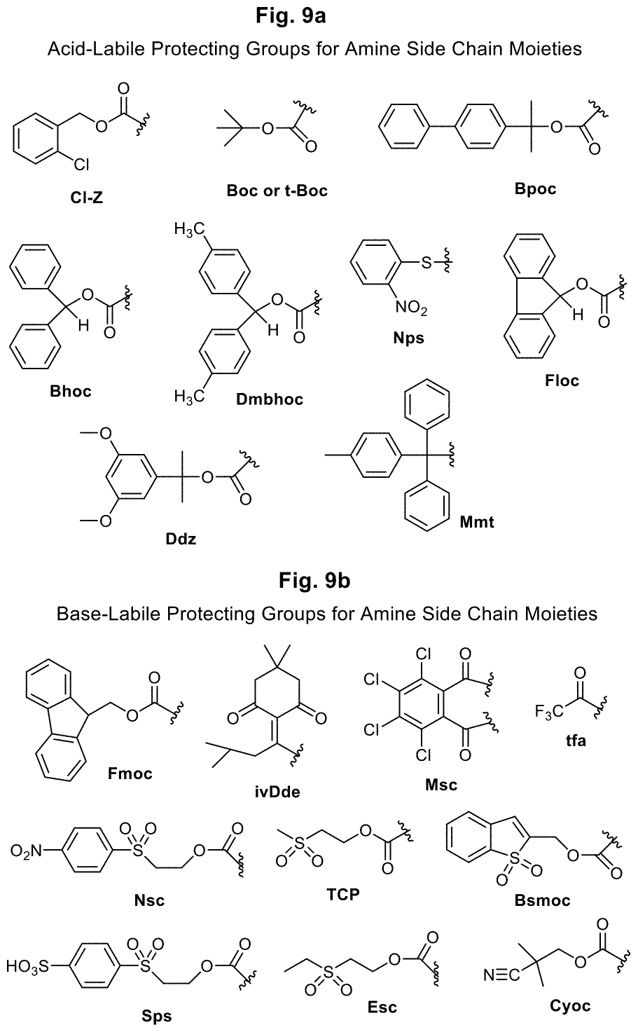

Again with reference to FIG. 7, in some embodiments, the α- or γ-substituents can be compositions of formula: IIIi (e.g., derived from 3-aminoalanine), IIIk (e.g., derived from 2,4-diaminobutanoic acid), IIIj (e.g., derived from ornithine), and/or IIIm (e.g., derived from lysine). These α- or γ-substituents all comprise an amine group. Consequently, the amine group of these substituents will typically comprise a protecting group. However, because this is a side chain protecting group generally remains intact during the entire synthesis of the PNA oligomer, this side chain protecting group can be orthogonal to the protecting group selected for the N-terminal amine (i.e. denoted Pg₁). Thus, if Pg₁ is base-labile, this side chain protecting group can be selected to be acid-labile or removed under conditions of neutral pH. A non-limiting list of such acid-labile amine side chain protecting groups is illustrated in FIG. 9a. These include, but are not limited to, Cl—Z, Boc, Bpoc, Bhoc, Dmbhoc, Nps, Floc, Ddz and Mmt.

Similarly, if Pg₁ is acid-labile, this side chain protecting group can be selected to be base-labile or removed under conditions of neutral pH. A non-limiting list of such base-labile amine side chain protecting groups is illustrated in FIG. 9b. These include, but are not limited to, Fmoc, ivDde, Msc, tfa, Nsc, TCP, Bsmoc, Sps, Esc and Cyoc.

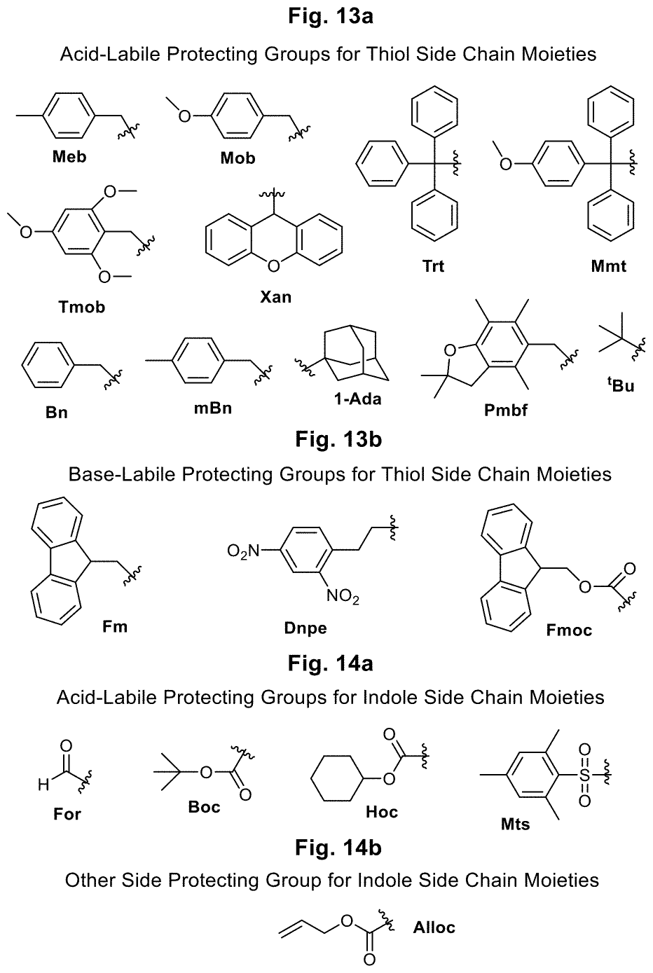

Again with reference to FIG. 7, in some embodiments, the α- or γ-substituents can be compositions of formula: IIIn (e.g., derived from cysteine), IIIo (e.g., derived from S-methyl-cysteine), and/or IIIp (e.g., derived from methionine). These α- or γ-substituents all comprise a sulfur atom. While it is not essential that compounds of formula IIIo or IIIp comprise a protecting group (but they can optionally be protected), thiol containing compounds of formula IIIn typically comprise a protecting group. However, because this side chain protecting group generally remains intact during the entire synthesis of the PNA oligomer, this side chain protecting group can be orthogonal to the protecting group selected for the N-terminal amine (i.e. Pg₁). Thus, if Pg₁ is base-labile, this side chain protecting group can be selected to be acid-labile or removed under conditions of neutral pH. A non-limiting list of such acid-labile protecting groups suitable for thiol containing side chain moieties is illustrated in FIG. 13a. These include, but are not limited to, Meb, Mob, Trt, Mmt, Tmob, Xan, Bn, mBn, 1-Ada, Pmbr and tBu.

Similarly, if Pg₁ is acid-labile, this side chain protecting group can be selected to be base-labile or removed under conditions of neutral pH. A non-limiting list of such base-labile protecting groups suitable for thiol containing side chain moieties is illustrated in FIG. 13b. These include, but are not limited to, Fm, Dnpe and Fmoc.

Again with reference to FIG. 7, in some embodiments, the α- or γ-substituents can be compositions of formula: IIIq (e.g., derived from serine), IIIr (e.g., derived from threonine), and/or IIIs (e.g., derived from tyrosine). These α- or γ-substituents all comprise a —OH (hydroxyl or phenol) group. Compounds of formulas IIIq, IIIr and IIIs will typically comprise a protecting group during PNA synthesis. However, because this is a side chain protecting group that generally remains intact during the entire synthesis of the PNA oligomer, this hydroxyl side chain protecting group can be orthogonal to the protecting group selected for the N-terminal amine (i.e. Pg₁).

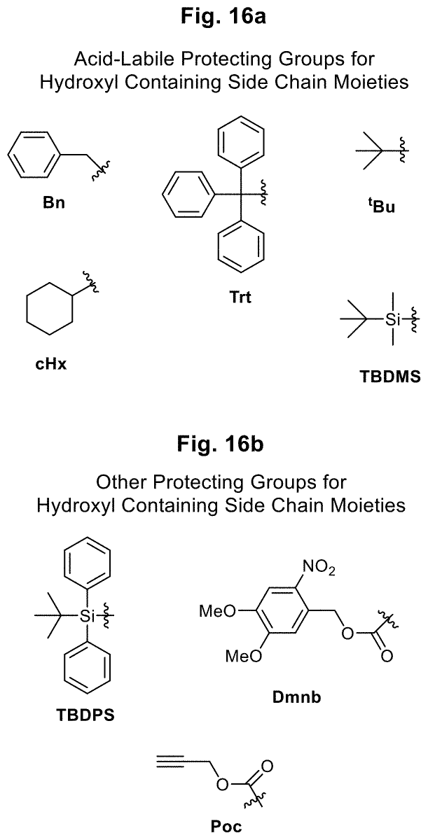

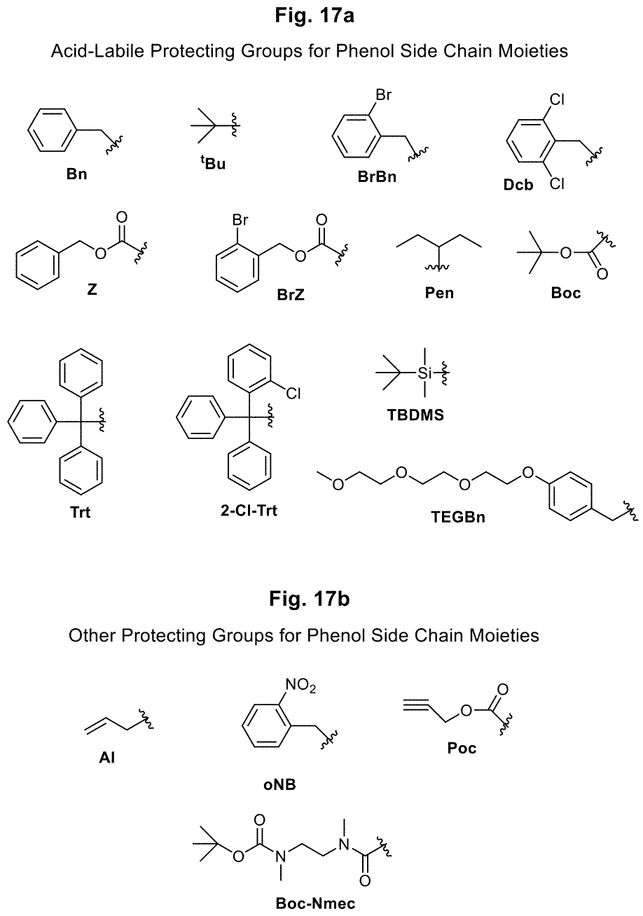

Thus, if Pg₁ is base-labile, the side chain protecting group can be selected to be acid-labile or removed under conditions of neutral pH. A non-limiting list of such acid-labile protecting groups suitable for hydroxyl containing moieties is illustrated in FIG. 16a. These include, but are not limited to, Bn, Trt, cHx, TBDMS and tBu. Because —OH of Tyrosine (Tyr) is phenolic, there is a potentially broader group of protecting group available. A non-limiting list of such acid-labile protecting groups for side chain moieties comprising a phenol is illustrated in FIG. 17a. These include, but are not limited to, Bn, tBu, BrBn, Dcb, Z, BrZ, Pen, Boc, Trt, 2-Cl-Trt and TEGBn.

Similarly, if Pg₁ is acid-labile, the side chain protecting group can be selected to be base-labile or removed under conditions of neutral pH. A non-limiting list of protecting groups for hydroxyl containing moieties that can be removed under conditions of neutral pH is illustrated in FIG. 16b. These include, but are not limited to, TBDPS, Dmnb and Poc. Because —OH of Tyrosine (Tyr) is phenolic, there is a potentially broader group of protecting group available. A non-limiting list of protecting groups for side chain moieties comprising a phenol that can be removed under conditions of neutral pH is illustrated in FIG. 17b. These include, but are not limited to, Al, oBN, Poc and Boc-Nmec.

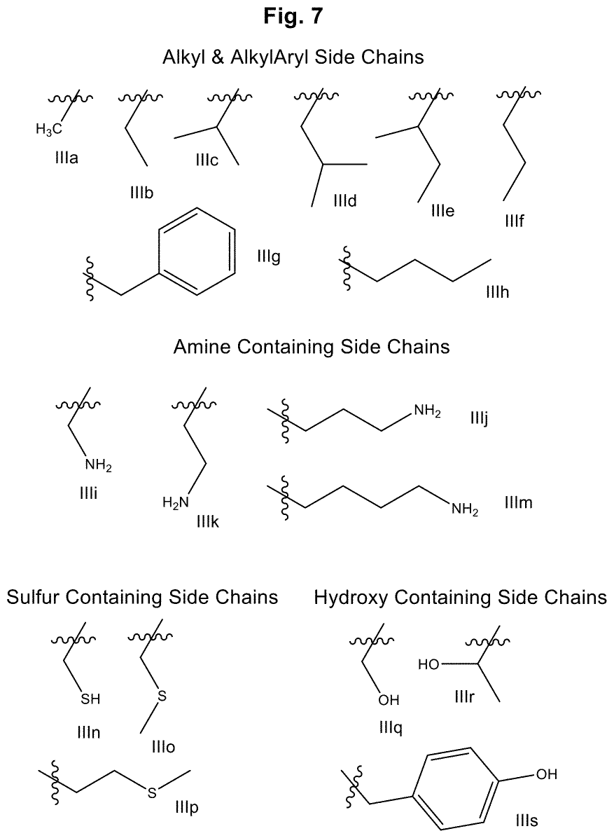

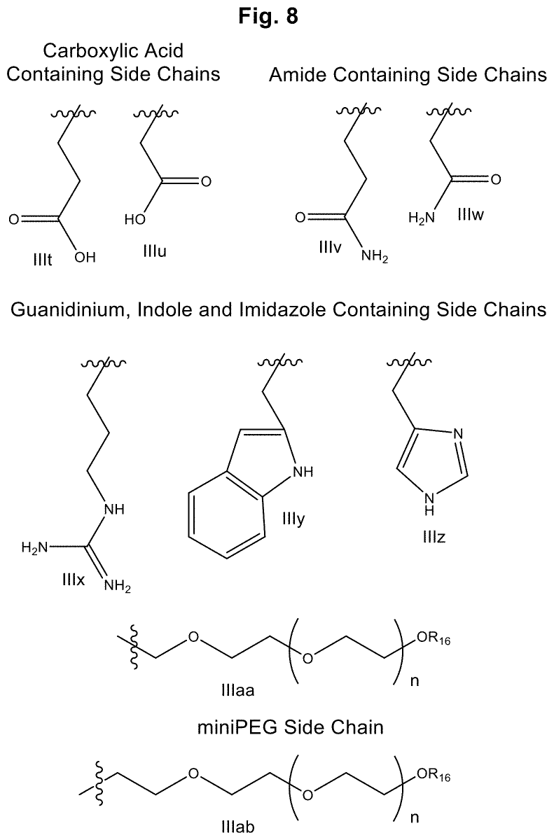

With reference to FIG. 8, in some embodiments, the α- or γ-substituents can be compositions of formula: IIIt (e.g., derived from glutamic acid) and/or IIIu (e.g., derived from aspartic acid). These α- or γ-substituents all comprise a —COOH (carboxylic) group. Compounds of formulas IIIt and IIIu will typically comprise a protecting group during PNA synthesis to thereby inhibit activation of the carboxylic acid group during the coupling reaction. However, because this is a side chain protecting group that generally remains intact during the entire synthesis of the PNA oligomer, this side chain protecting group can be orthogonal to the protecting group selected for the N-terminal amine (i.e. Pg₁).

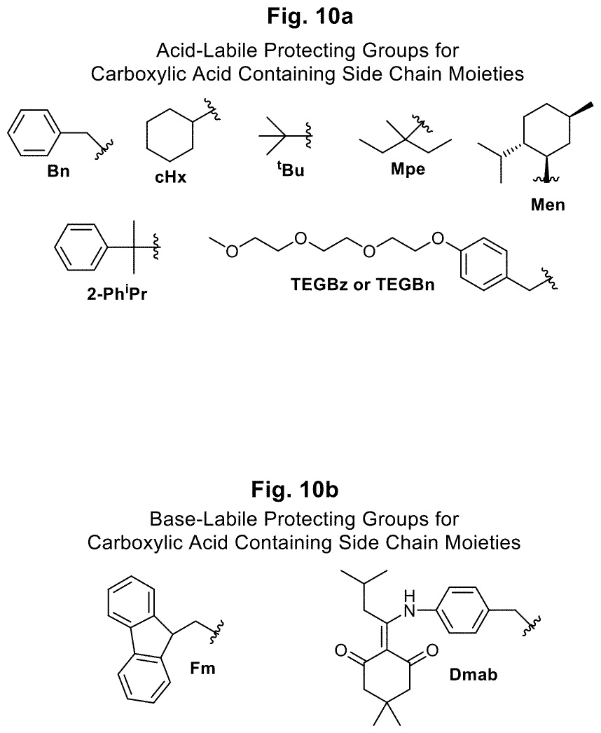

Thus, if Pg₁ is base-labile, the side chain protecting group can be selected to be acid-labile or removed under conditions of neutral pH. A non-limiting list of such acid-labile protecting groups suitable for use with carboxylic acid containing side chain moieties is illustrated in FIG. 10a. These include, but are not limited to, Bn, cHx, tBu, Mpe, Men, 2-PhⁱPr and TEGBz.

Similarly, if Pg₁ is acid-labile, the side chain protecting group can be selected to be base-labile or removed under conditions of neutral pH. A non-limiting list of such base-labile protecting groups suitable for use with carboxylic acid containing side chain moieties is illustrated in FIG. 10b. These include, but are not limited to, Fm and Dmab.

With reference to FIG. 8, in some embodiments, the α- or γ-substituents can be compositions of formula: IIIy (e.g., derived from glutamine) and/or IIIw (e.g., derived from asparagine). These α- or γ-substituents all comprise a —C(═O)NH₂ (amide) group. Compounds of formulas IIIy and IIIw do not necessarily require a protecting group during PNA synthesis but nevertheless, standard protecting groups used in peptide synthesis can be used. When used, this side chain protecting group can be orthogonal to the protecting group selected for the N-terminal amine (i.e. Pg₁).

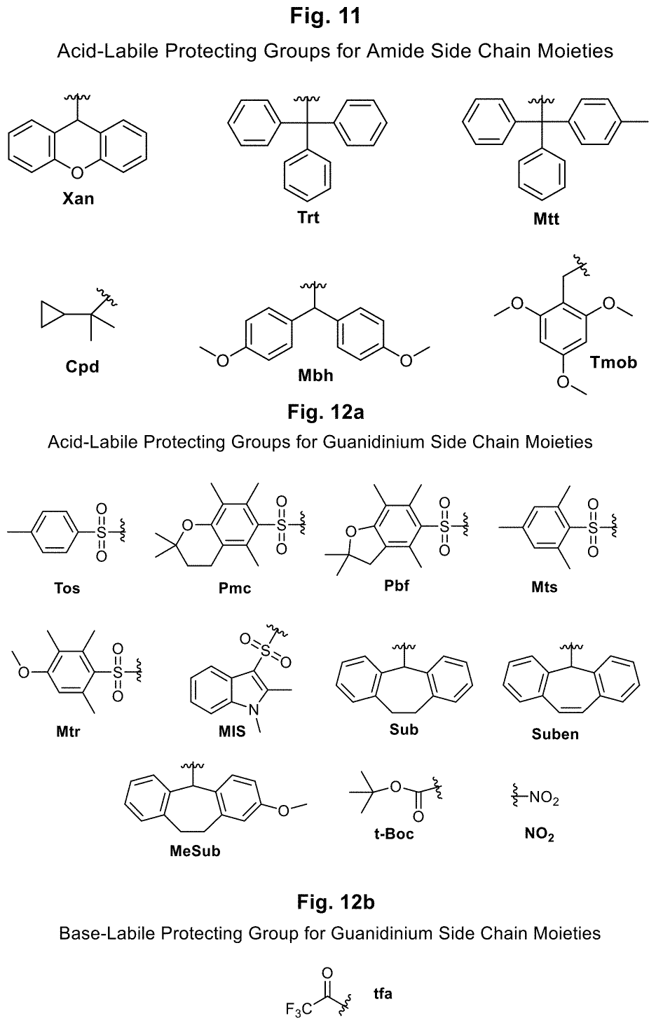

Thus, if Pg₁ is base-labile, the side chain protecting group can be selected to be acid-labile or removed under conditions of neutral pH. A non-limiting list of such acid-labile protecting groups for amide containing side chain moieties is illustrated in FIG. 11. These include, but are not limited to, Xan, Trt, Mtt, Cpd., Mbh and Tmob. Similarly, if Pg₁ is acid-labile, the side chain protecting group can be selected to be base-labile or removed under conditions of neutral pH.

With reference to FIG. 8, in some embodiments, the α- or γ-substituents can be compositions of formula: IIIx (e.g., derived from arginine (Arg)—and containing a guanidinium moiety), IIIy (e.g., derived from tryptophan (Trp)—and containing an indole moiety) and/or IIIz (e.g., derived from histidine (His)—and containing an imidazole moiety). Compounds of formulas IIIx, IIIy and IIIz will typically comprise a protecting group during PNA synthesis. However, because this side chain protecting group generally remains intact during the entire synthesis of the PNA oligomer, this side chain protecting group can be orthogonal to the protecting group selected for the N-terminal amine (i.e. Pg₁)

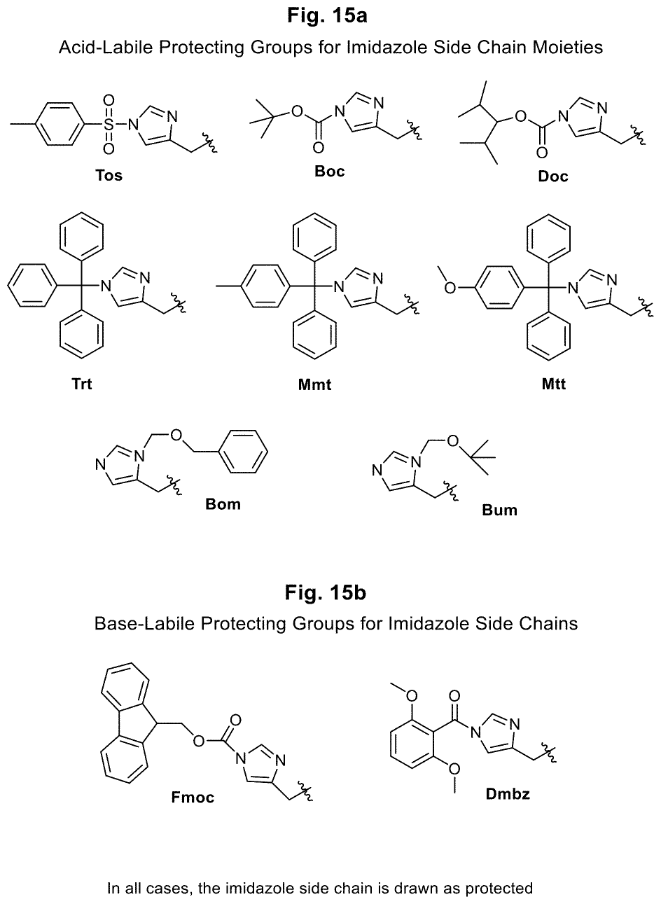

Thus, if Pg₁ is base-labile, the side chain protecting group can be selected to be acid-labile or removed under conditions of neutral pH. A non-limiting list of such acid-labile side chain protecting groups suitable for use with guanidinium containing side chain moieties is illustrated in FIG. 12a. These include, but are not limited to, Tos, Pmc, Pbf, Mts, Mtr, MIS, Sub, Suben, MeSub, Boc and NO₂. A non-limiting list of such acid-labile side chain protecting groups suitable for use with indole containing side chain moieties is illustrated in FIG. 14a. These include, but are not limited to, For, Boc, Hoc and Mts. A non-limiting list of such acid-labile side chain protecting groups suitable for use with imidazole containing side chain moieties is illustrated in FIG. 15a. These include, but are not limited to, Tos, Boc, Doc, Trt, Mmt, Mtt, Bom and Bum.

Similarly, if Pg₁ is acid-labile, the side chain protecting group can be selected to be base-labile or removed under conditions of neutral pH. A non-limiting list of such base-labile side chain protecting groups suitable for use with guanidinium containing side chain moieties is illustrated in FIG. 12b. These include, but are not limited to, tfa. A non-limiting list of such side chain protecting groups removable under conditions of neutral pH suitable for use with indole containing side chain moieties is illustrated in FIG. 14b. These include, but are not limited to, Alloc. A non-limiting list of such base-labile side chain protecting groups suitable for use with imidazole containing side chain moieties is illustrated in FIG. 15b. These include, but are not limited to, Fmoc and Dmbz.

In some embodiments, the α- or γ-substituents (i.e. side chains) can be a moiety of formula IIIaa (a.k.a. a miniPEG side chain):

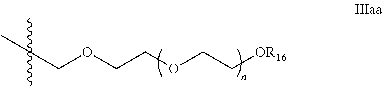

wherein, R₁₆ is selected from H, D and C₁-C₄ alkyl group; and n can be a whole number from 0 to 10, inclusive. For example, see Refs A-5 and B-5. In some embodiments, the α- or γ-substituents (i.e. side chains) can be a moiety of formula IIIab:

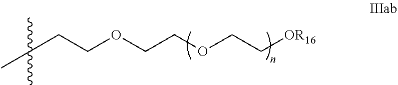

wherein, R₁₆ is selected from H, D and C₁-C₄ alkyl group; and n can be a whole number from 0 to 10, inclusive. Side chains of this formula can be produced in the same manner as exemplified in Refs A-5 and B-5, except that substitution of homoserine instead of serine starting materials will produce backbone moieties comprising the formula IIIab instead of formula IIIaa

VIII. Ethyl Esters Capable of Specific Removal

As discussed in the introduction, PNA monomers are often prepared by saponification (using a strong base) of the ester group of a fully protected PNA monomer ester. However, where the PNA monomer ester comprises a base-labile protecting group on either the N-terminal amine group, or a nucleobase protecting group, that base-labile protecting group is always at least partially deprotected under these conditions; leading (in Applicants' experiences) to poor yields and poor quality (i.e. impure) products that require column chromatography to achieve an adequate level of purity for use in PNA oligomer synthesis.

To avoid use of TFA during each synthetic cycle and because of its compatibility with amino acid synthesis, Fmoc is the most common group used as Pg₁ in PNA monomer preparation. Consequently, saponification of the ester group of a PNA monomer ester comprising Fmoc as Pg₁ results in significant generation of dibenzofulvene (the product of base-induced removal of Fmoc) and at least some PNA monomer comprising no N-terminal amine protecting group. These impurities should be removed (especially the PNA monomer comprising no N-terminal amine protecting group) before the PNA monomer is used in PNA synthesis. In Applicants experience, monomer purity and particularly yield is may be negatively affected as the PNA monomer becomes more water soluble. Simply stated, the ester group of the PNA monomer ester is not orthogonally protected if other protecting groups are removed when the ester is removed to produce the PNA monomer. The generation of unwanted impurities may lower yield and complicate the purification of products.

To avoid the complications associated with this approach, Applicants sought to find a truly orthogonal protection scheme whereby the ester group of the PNA monomer could be removed without significant removal of any of the other protecting groups used in the PNA monomer (i.e. the protecting group used as Pg₁ or any nucleobase protecting groups). Accordingly, this ester should be stable to conditions that can be used to remove the acid-labile and base-labile protecting groups associated with peptide and PNA synthesis. To this end, PNA monomer esters of the general formula II (herein referred to as PNA Monomer Esters) meet these criteria. Thus, in some embodiments, this invention pertains to a PNA Monomer Ester that is a compound of general formula II:

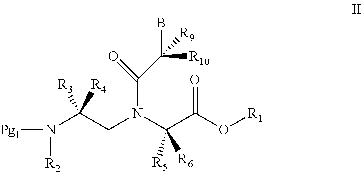

or a pharmaceutically acceptable salt thereof, wherein, B is a nucleobase, optionally comprising one or more protecting groups (See, e.g., Section 4(VI), above for a discussion of nucleobase protecting groups); Pg₁ is an amine protecting group and R₁ is a group of formula I;

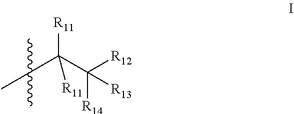

wherein, each R₁₁ is independently H, D, F, C₁-C₆ alkyl, C₃-C₆ cycloalkyl or aryl; each R₁₂, R₁₃ and R₁₄ is independently selected from H, D, F, Cl, Br and I, provided however that at least one of R₁₂, R₁₃ and R₁₄ is independently selected from Cl, Br and I. With respect to formula II, R₂ can be H, D or C₁-C₄ alkyl; each of R₃, R₄, R₅, and R₆ can be independently selected from the group consisting of: H, D, F, and a side chain selected from the group consisting of: IIIa, IIIb, IIIc, IIId, IIIe, IIIf, IIIg, IIIh, IIIi, IIIj, IIIk, IIIm, IIIn, IIIo, IIIp, IIIg, IIIr, IIIs, IIIt, IIIu, IIIy, IIIw, IIIx, IIIy, IIIz, IIIaa and IIIab, wherein each of IIIi, IIIj, IIIk, IIIm, IIIn, IIIo, IIIp, IIIg, IIIr, IIIs, IIIt, IIIu, IIIy, IIIw, IIIx, IIIy, and IIIz independently and optionally comprises a protecting group (See, e.g., Section 4(VII), above, for a discussion of various amino acid side chain protecting groups);

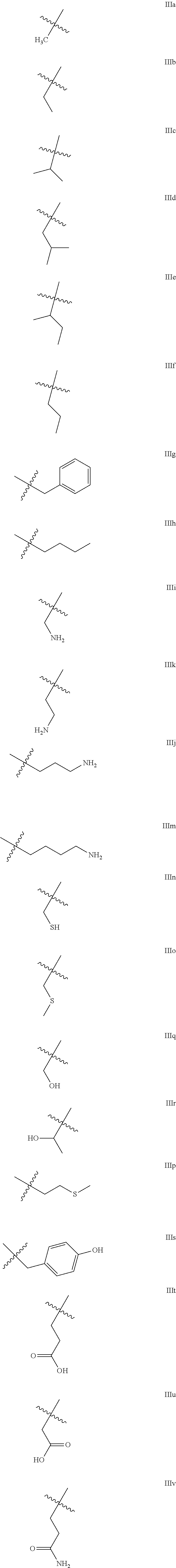

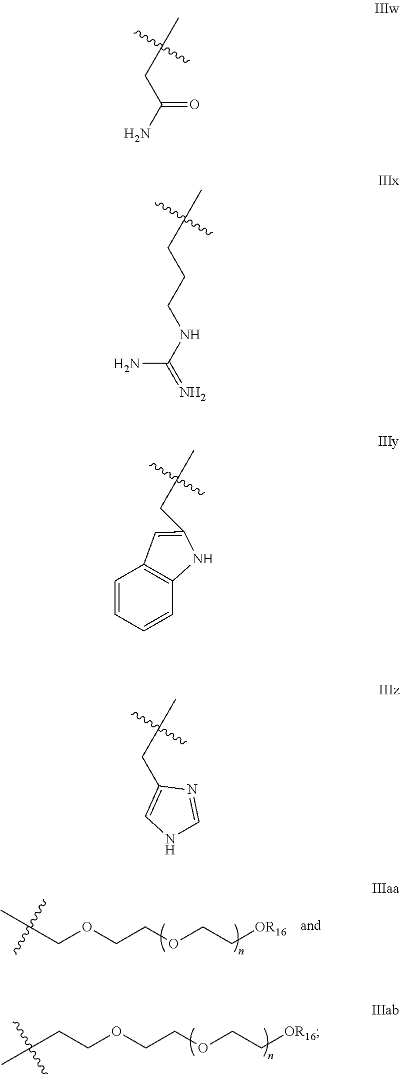

each of R₉ and R₁₀ can be independently selected from the group consisting of: H (hydrogen), D (deuterium) and F (fluorine); R₁₆ can be selected from H, D and C₁-C₄ alkyl group; and n can be a whole number from 0 to 10, inclusive.

In some embodiments, B is a naturally occurring nucleobase or a nonnaturally occurring nucleobase. In some embodiments, B is a modified nucleobase. In some embodiments, B is an unmodified nucleobase. In some embodiments, B is selected from the group consisting of: adenine, guanine, thymine, cytosine, uracil, pseudoisocytosine, 2-thiopseudoisocytosine, 5-methylcytosine, 5-hydroxymethyl cytosine, xanthine, hypoxanthine, 2-aminoadenine (a.k.a. 2,6-diaminopurine), 2-thiouracil, 2-thiothymine, 2-thiocytosine, 5-chlorouracil, 5-bromouracil, 5-iodouracil, 5-chlorocytosine, 5-bromocytosine, 5-iodocytosine, 5-propynyl uracil, 5-propynyl cytosine, 6-azo uracil, 6-azo cytosine, 6-azo thymine, 7-methylguanine, 7-methyladenine, 8-azaguanine, 8-azaadenine, 7-deazaguanine, 7-deazaadenine, 7-deaza-2-aminoadenine (7-deaza-diaminopurine), 3-deazaguanine, 3-deazaadenine, 7-deaza-8-aza guanine, 7-deaza-8-aza adenine, 5-propynyl uracil and 2-thio-5-propynyl uracil, including tautomeric forms of any of the foregoing.

In some embodiments of the group of formula I, each of R₁₁ is the same. In some embodiments of the group of formula I, each of R₁₁ is different. With respect to formula I, one of R₁₂, R₁₃ and R₁₄ is selected from chlorine (Cl), bromine (Br) and iodine (I). Without being bound by theory, the mechanism as described by Hans et al. (See: Ref. C-7) for removal of groups of formula I involves an ‘oxidation-reduction condensation’ whereby reaction of said chlorine (Cl), bromine (Br) or iodine (I) atom as R₁₂, R₁₃ or R₁₄ with a metal (such as zinc) or organophosphine (for example: linear, branched, and cyclic trialkylphosphines, such as trimethylphosphine, triethylphosphine, tri-n-propylphosphine, tri-n-butylphosphine, triisopropylphosphine, triisobutylphosine, and tricyclohexylphosphine; Aryl and arylalkyl substituted phosphines such as tribenzylphosphine, diethylphenylphosphine, dimethylphenylphosine; and phosphorous triamides such as hexamethylphosphorous triamide, and hexaethylphoshorous triamide) results in abstraction of said chlorine (Cl), bromine (Br) or iodine (I) to form a salt. This reaction causes removal of the ester protecting group of formula I from the PNA Monomer Ester and results in production of the carboxylic acid (for our purposes conversion of a PNA Monomer Ester to a PNA monomer). The reaction can be carried out without needing to go to extremes of pH that might cause removal of Pg₁ or an exocyclic nucleobase protecting group. Of course, because this reaction involves and oxidation-reduction reaction, protecting groups that are labile to oxidizing or reducing conditions should generally be avoided. However, it should not go unsaid that compounds of formula II can still be subjected to the more common ester saponification procedures (i.e. treatment with lithium hydroxide or sodium hydroxide) when it is determined that there are unwanted side reactions that occur by subjecting the PNA Monomer Ester to oxidizing or reducing conditions. Applicants have also surprisingly observed that the protecting groups of Formula I are substantially stable to at least mildly reducing conditions, such as treatment with sodium cyanoborohydride.

In some embodiments, two of R₁₂, R₁₃ and R₁₄ are independently selected from chlorine (Cl), bromine (Br) and iodine (I). In some embodiments, all three of R₁₂, R₁₃ and R₁₄ are independently selected from chlorine (Cl), bromine (Br) and iodine (I). In some embodiments, each of R₁₂, R₁₃ and R₁₄ is chlorine (Cl). In some embodiments, each of R₁₂, R₁₃ and R₁₄ is bromine (Br). In some embodiments, one of R₁₂, R₁₃ and R₁₄ is iodine (I) and the others of R₁₂, R₁₃ and R₁₄ are H. In some embodiments, one of R₁₂, R₁₃ and R₁₄ is bromine (Br) and the others of R₁₂, R₁₃ and R₁₄ are H.

2,2,2-trichloroethanol, 2,2,2-tribromoethanol and 2-iodoethanol are commercially available as starting materials. The present disclosure demonstrates that the 2,2,2-trichloroethyl ester (TCE), 2,2,2-tribromoethyl ester (TBE) and 2-iodoethyl ester (2-IE) can be efficiently removed to produce desired PNA monomers in good yield and high purity. In at least one case, the PNA monomer purity was found to be greater than 99.5% pure by HPLC analysis at 260 nm. This however is not intended to be a limitation as all moieties of formula I should be reactive. The use of 2,2,2-trichloroethyl- and/or 2,2,2-tribromoethyl-groups as protecting groups have been reported in at least the following publications (See: A-2, A-3, C-2, C-4, C-6, C-7, C-14, C-16, C-23, C-25, C-28 and C-29), but none of which relate to their use as an orthogonal protecting group for the C-terminal ester of a PNA monomer.

IX. Synthesis of a Backbone and other Compositions Containing the Specified Esters

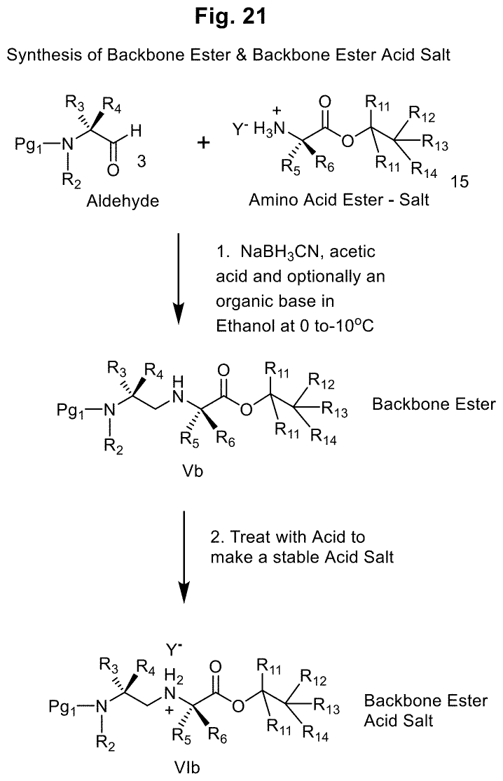

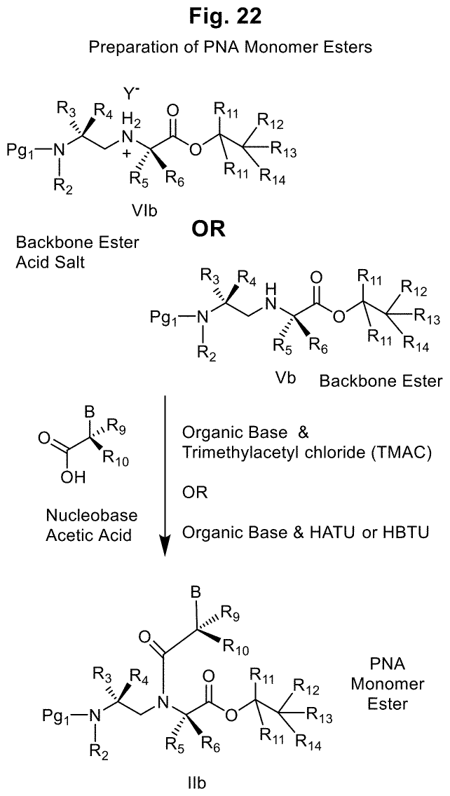

Though not intending to be limiting, it has been determined that (with reference to FIG. 21) suitable Backbone Esters and Backbone Ester Acid Salts that can be used for the synthesis of PNA Monomer Esters (See: FIG. 22) can be prepared by reductive amination from a suitably selected aldehyde (Formula 3) and a suitably selected amino acid ester salt (Formula 15). Most advantageously, each aldehyde (Formula 3) and each amino acid ester salt (Formula 15) can itself be derived from naturally and non-naturally occurring amino acids. Even the miniPEG side chain of formula IIIaa can be derived from the amino acid serine (See: Ref A-5 and B-5) and side chain moieties of formula IIIab can be derived from the amino acid homoserine. Accordingly, by judicious selection of the correct starting materials, one or more of groups R₃, R₄, R₅ and R₆ can be a group of formula: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIv, IIIw, IIIx, IIIy, IIIz, IIIaa and IIIab. Deuterated amino acid starting materials are also commercially available. Fluorinated amino acids can also be prepared (See: Ref. C-10). These are all considered as suitable starting materials for use in the process described below.

a) Preparation of Amino Acid Esters and Amino Acid Ester Salts

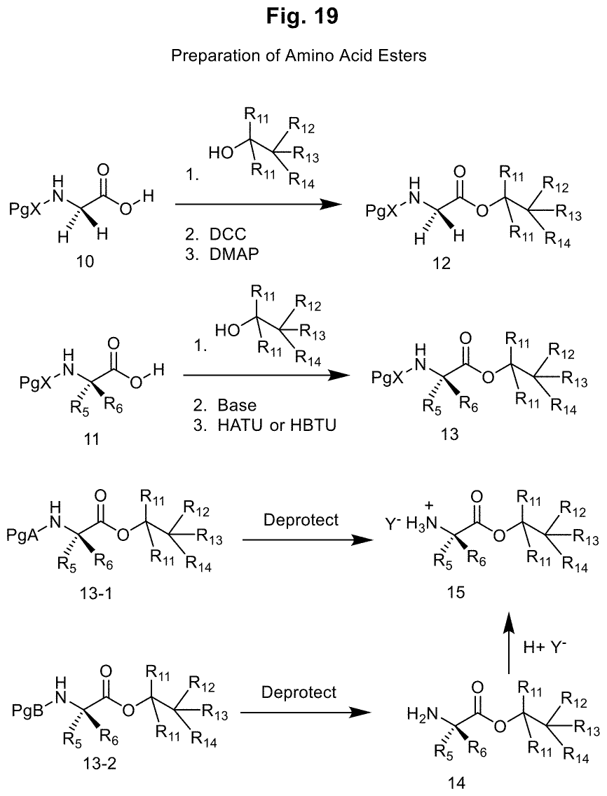

With reference to FIG. 19, a suitable process for converting amino acids to protected amino acid esters and then finally to amino acid ester salts is illustrated. In some embodiments, a compound of formula 10 is the amino acid glycine that is N-protected with an acid-labile or base-labile protecting group PgX. Because glycine is achiral, there is no concern regarding epimerization. Accordingly, the ester of the protected glycine can be efficiently prepared by reaction of 10 with an alcohol (ethanol derivative) of formula Ia:

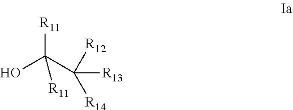

(wherein, R₁₁, R₁₂, R₁₃ and R₁₄ are defined as for formula II). In some embodiments, the reaction is carried out in an aprotic organic solvent such as DCM in the presence of at least one equivalent of DCC (or EDC) and a catalytic amount of DMAP (See: Example 1). With reference to FIG. 19, an N-protected glycine ester compound of formula 12 is produced.

This process will also work for chiral amino acids but is well-known to cause epimerization of the chiral center leading to degradation of the chiral purity of the amino acid products. For this reason, when the ester of a chiral amino acid is wanted, a carboxylic activating agent that is known to avoid (or at least minimize) epimerization of the chiral center is preferred. The carboxylic acid activating reagents (also known as coupling agents) HATU and HBTU are well known in peptide chemistry to activate carboxylic acids to nucleophilic attack whilst maintaining chiral purity of the amino acid. Accordingly, with reference to FIG. 19, when the ester of an N-protected chiral amino acid (i.e. compounds of formula 13) is desired as the product, a N-protected chiral amino acid compound of formula 11 can be reacted with an alcohol of formula Ia, in the presence of at least on equivalent of organic base (such as TEA, NMM or DIPEA) and at least one equivalent of HATU or HBTU. With reference to FIG. 19, an N-protected ester of the desired chiral amino acid (i.e. compound of formula 13) is produced (See: Example 2). For the avoidance of doubt, the groups R₅ and R₆ can comprise the appropriate side chain protecting groups (including natural amino acid side chains) as described herein.

Production of the Backbone Ester and Backbone Ester Acid Salt compounds as illustrated in FIG. 21, may employ compounds wherein the free N-terminal amine is protonated (i.e. compounds of formula 15). It is also worth noting that the acid salt of the free amine (i.e. the protonated amine group) is more stable as compared with the free amino acid ester (i.e. compound of formula 14—that, for example, can react with itself by attach of the amine on the ester to form dimers, trimers, etc.). With reference to FIG. 19, PgX can be an acid-labile protecting group (PgA—compound of formula 13-1) or a base-labile protecting group (PgB—compound of formula 13-2). Accordingly, with reference to FIG. 19, if the N-amine protecting group is acid-labile (PgA—compound of formula 13-1), deprotection will generally provide the N-terminal amine as its acid salt (i.e. compound of formula 15—See: Example 3). Alternatively, if the N-amine protecting group is base-labile (PgB—compound of formula 13-2), deprotection will generally provide the free amine (i.e. compound of formula 14) that can be converted to the acid salt (i.e. compound of formula 15) by treatment with an acid (See: Example 4). Suitable acids include, but are not limited to, hydrochloric acid (HCl), hydrobromic acid (HBr), hydroiodic acid (HI), acetic acid, trifluoroacetic acid and citric acid, wherein Y⁻ is the counterion Cl⁻, Br⁻, I⁻, AcO⁻, CF₃CO₂⁻ and the anion of citric acid.

Consequently, from the forgoing is should be apparent that by following the disclosure provided herein, any amino acid ester salt according to formula 15:

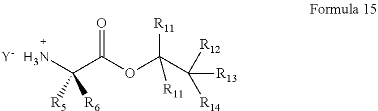

can be prepared using the procedures disclosed herein, wherein Y⁻, R₅, R₆, R₁₁, R₁₂, R₁₃ and R₁₄ are as defined herein.

b) Preparation of Aldehydes

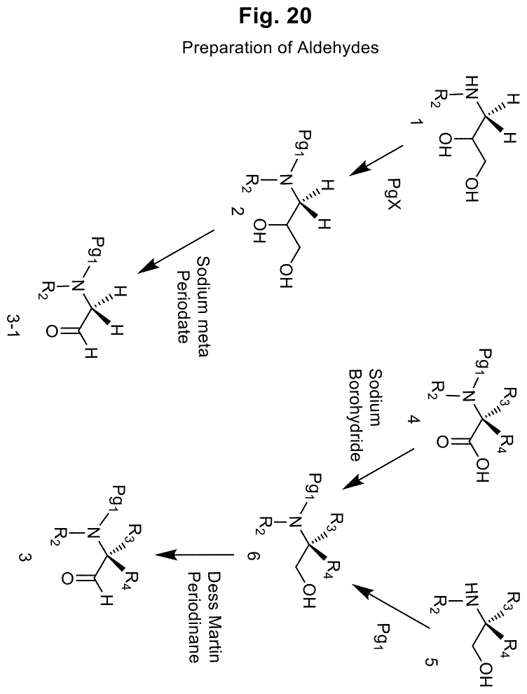

With reference to FIG. 20, methods for the preparation of aldehydes suitable for the production of Backbone Esters and Backbone Ester Acid Salts are illustrated. Without being bound by theory, an effective current route to the glycine equivalent of the aldehyde (the achiral version—Formula 3-1) is by protecting the amino group of the 3-amino-1,2-propanediol (Formula 1) with the appropriate protecting group Pg₁ (which as defined above can be an acid-labile protecting group (e.g. Boc) or a base-labile protecting group (e.g. Fmoc)) to thereby produce the N-protected 3-amino-1,2-propanediol (compound of formula 2—See: Example 5). The N-protected 3-amino-1,2-propanediol (formula 2) can then be oxidized to the aldehyde (compound of formula 3-1) by treatment with excess sodium meta periodate (NaIO₄) by treatment in a biphasic (aqueous and organic solvent mix) system at or below room temperature (See: Example 5). In our hands, this process produces very clean aldehyde product (compound 3-1) in high yield.

With reference to FIG. 20, there are several routes to the aldehydes (chiral and achiral) according to formula 3, by use of amino acids and their related amino alcohols. N-protected amino acids illustrated by formula 4 are commercially available from numerous commercial sources of peptide synthesis reagents. From these same commercial sources, amino alcohols of structure according to formula 5 and N-protected amino alcohols of structure according to formula 6 can be purchased (See: Chem Impex online catalog and Bachem online catalog).

When not commercially available, amino alcohols of structure according to formula 5 can be prepared directly from an amino acid as described, for example, by Ramesh et al. (Ref. C-20) and Abiko et al. (Ref. C-1). Amino alcohols of structure according to formula 5 can then be converted to N-protected amino alcohols according to formula 6 by reaction with the desired amine protecting group (Pg₁—See: Example 6).

Alternatively, there are numerous reports of converting N-protected amino acids (accordingly to formula 4) into their counterpart N-protected amino alcohols (according to formula 6). For example, that conversion can be accomplished using sodium borohydride reduction of the first formed mixed anhydride according to the procedure reported by Rodriguez et al. (Ref. C-21 and See: Example 7). Albeit with different reagents and protecting group strategies, the conversion N-protected amino acids of formula 4 into their corresponding N-protected amino alcohols according to formula 6 has been frequently described in the scientific literature (See: Refs. C-1, C-3, C-5, C-15 and C-24). Taken together, these reports, and the information provided herein, provides access to virtually any desired N-protected amino alcohol according to formula 6, wherein R₃ and R₄ are defined herein (in side chain protected or side chain deprotected form).

With reference to FIG. 20, any N-protected amino alcohol according to formula 6 can then be converted to an N-protected amino aldehyde according to formula 3. There are several literature preparations useful for converting an N-protected amino alcohol according to formula 6 into a corresponding N-protected amino aldehyde according to formula 3 (See for example: Refs. C-12 and C-26, C-30, C-32-C-33 and C-35). There is concern that epimerization can occur during conversion of the alcohol to an aldehyde. For this reason, Applicants have elected follow the procedure of Myers et al. (Ref. C-18) wherein Dess-Martin Periodinane as the oxidizing agent and wet DCM (Ref. C-17) are used because this procedure is reported to be superior for retention of chiral purity (See: Example 8). Indeed, the data provided in the Examples below demonstrates that Backbone Esters and Backbone Ester Acid Salts of high optical purity can be obtained. There is also a recent report whereby N-protected amino acids of formula 4 were converted directly to their corresponding N-protected amino aldehyde compounds of formula 3 (See: Ref. C-12).

Consequently, from the forgoing is should be apparent that by following the disclosure provided herein, any N-protected aldehyde according to formula 3:

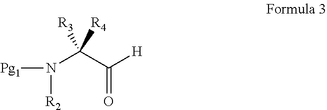

can be prepared, wherein Pg₁, R₂, R₃, and R₄ are as defined herein.

c) Combining the Amino Acid Esters and the Aldehydes to Form a Backbone Ester or Backbone Ester Acid Salt

With reference to FIG. 21, an N-protected aldehyde according to formula 3 is reacted with an amino acid ester salt according to formula 15 under conditions suitable for performing a reductive amination to thereby produce a Backbone Ester according to formula Vb:

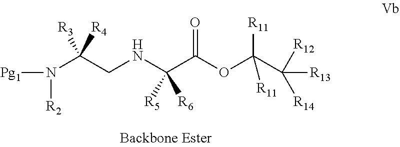

wherein Pg₁, R₂, R₃, R₄, R₅, R₆, R₁₁, R₁₂, R₁₃ and R₁₄ are defined herein.

Contrary to the reports from Salvi et al. (Ref. C-22), Applicants were able to produce the desired product (See: Example 9) when reacting N-Fmoc-aminoacetaldehyde with either the TBE or TCE esters of glycine as their TFA salts (Table 9B); albeit in less than remarkable yield (which yield has been improved upon by subsequent examination—See Example 9B & 9C). In order to reduce the prevalence of the bis-aldehyde adduct, the reaction may be cooled to 0° C. or less (for example to −15° C. to −10° C.) and ethanol may be used as the solvent. The pH of the reaction could be monitored (e.g., by pH paper) and generally maintained in the range of 3-5 (optimal for sodium cyanoborohydride) by the addition of excess carboxylic acid (e.g., acetic acid). For those reactions performed in Example 9, sodium cyanoborohydride was used as the reducing agent. Although the reaction was performed under reducing conditions, there did not appear to be any evidence of direct reaction between the cyanoborohydride reducing agent and the TCE or TBE esters. Thus, it somewhat surprisingly appears that amino acid ester salt according to formula 15 is stable under certain types of reducing conditions such that these esters can be useful for the production of Backbone Esters of formula Vb.

A reductive amination reaction has at least been twice reported to be successful in producing PNA monomers (See: Refs. C-8 and C-9). These reports are not however inconsistent with Salvi et al. who reported limited success if the aldehyde was substituted (Ref. C-8 used a protected glutamic acid side chain in the aldehyde and Ref. C-9 used a protected lysine side chain in the aldehyde).

In Applicants experience, a Backbone Ester according to formula Vb can be fairly unstable and may exhibit decomposition, even when stored overnight in a refrigerator or freezer. Without intending to be bound to any theory, it is believed that the presence of a secondary amine in compounds of formula Vb may lead to both Fmoc migration (from the primary to the secondary amine) and also loss of the base-labile Fmoc protecting group because of the basicity of the secondary amine. Again, without intending to be bound to any theory, it is also possible that the Backbone Ester cyclizes to form a ketopiperazine by attack of the protected amine on the ester group.

Regardless, a Backbone Ester according to formula Vb can be used immediately or in some embodiments they can be reacted with a suitable acid to form its corresponding acid salt (i.e. a Backbone Ester Acid Salt of formula VIb) as illustrated in FIG. 21 (See also Example 9).

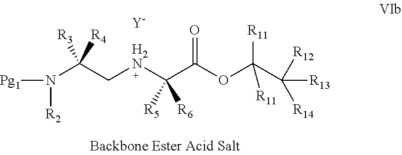

wherein Pg₁, Y⁻, R₂, R₃, R₄, R₅, R₆, R₁₁, R₁₂, R₁₃ and R₁₄ are defined herein.

Applicants have found such Backbone Ester Acid Salts to be solids that are easily weighted out and handled and they appear to be stable over long periods. Suitable salts of the amine that can be prepared include; hydrochloride salts, hydrobromide salts, hydroiodo salts, acetate salts, trifluoroacetate salts, tosylate salts, citrate salts, etc. In some embodiments, the salt is a tosylate salt (formed by addition p-toluenesulfonic acid (usually as its monohydrate—See: Example 9C).

d) Preparation of PNA Monomer Esters

With the Backbone Ester and/or Backbone Ester Acid Salt available, production of a PNA Monomer Ester may be carried out using well developed procedures (See Refs A-1 to A-5 and B-1 to B-5). With reference to FIG. 22, the carboxylic acid group of the nucleobase acetic acid is activated to nucleophilic displacement. Numerous methods are available and known in the art. However, FIG. 22 illustrates two (non-limiting) options.

In some embodiments, the carboxylic acid group of the nucleobase acid (e.g., a nucleobase acetic acid) can be activated by formation of a mixed anhydride. For example, a nucleobase acetic acid can be treated with an organic base (such as NMM, TEA or DIPEA—generally in excess) and at least one equivalent of trimethylacetyl chloride (TMAC) to thereby form a mixed anhydride as an intermediate. Once formed, the mixed anhydride intermediate can be reacted with either the Backbone Ester (formula Vb) or, so long as enough organic base is present to deprotonate it, the Backbone Ester Acid Salt (formula VIb). The secondary amine of the Backbone Ester (including Backbone Ester generated by in situ deprotonation of the Backbone Ester Acid Salt) can then react with the mixed anhydride to form the PNA Monomer Ester (formula IIb—See: Example 10).

Alternatively, in some embodiments, the nucleobase acid (e.g., a nucleobase acetic acid) is treated with an organic base (usually in excess) and at least one equivalent of activating agent such as HATU or HBTU to form an activated intermediate. Once formed, the activated intermediate can be reacted with either the Backbone Ester (formula Vb) or, so long as enough organic base is present to deprotonate it, the Backbone Ester Acid Salt (formula VIb). The secondary amine of the Backbone Ester (including Backbone Ester generated by in situ deprotonation of the Backbone Ester Acid Salt) can then react with the activated intermediate to form the PNA Monomer Ester (formula IIb).

The nucleobase acids can be protected or unprotected but generally they are protected if they possess a functional group that can interfere with: (i) the chemistry used to produce the PNA Monomer Ester; (ii) the chemistry used to manufacture the PNA oligomer; or (iii) the conditions used to deprotect and work up the PNA oligomer (post synthesis).

These PNA monomer preparation reactions are generally carried out in an aprotic organic solvent. Some non-limiting examples of suitable solvents include: ACN, THF, 1,4-dioxane, DMF, and NMP.

e) Synthesis of a PNA Monomer from a PNA Monomer Ester

There are numerous reports of using the TCE and TBE groups as protecting groups (See for example: Refs. C-2, C-4, C-6, C-7, C-11, C-14, C-16, C-23, C-25, C-28 and C-29). However, given the unique properties, protecting group strategies and complex synthesis protocols involved in PNA monomer synthesis, it is not apparent from these references that the TCE, TBE, 2-IE and/or 2-BrE sters could be successfully used to produce PNA Monomer Esters (of formula II or IIb) or that said PNA Monomer Esters could be used to so cleanly produce PNA monomers suitable for use in PNA oligomer synthesis. Further, the data presented in the Examples below demonstrates (somewhat unexpectedly given their complexity and the lack of any relevant discussion in the literature) that use of PNA Monomer Esters comprising TCE, TBE and/or 2-IE ester groups can produce PNA monomers in high yield, high purity, including high optical purity.

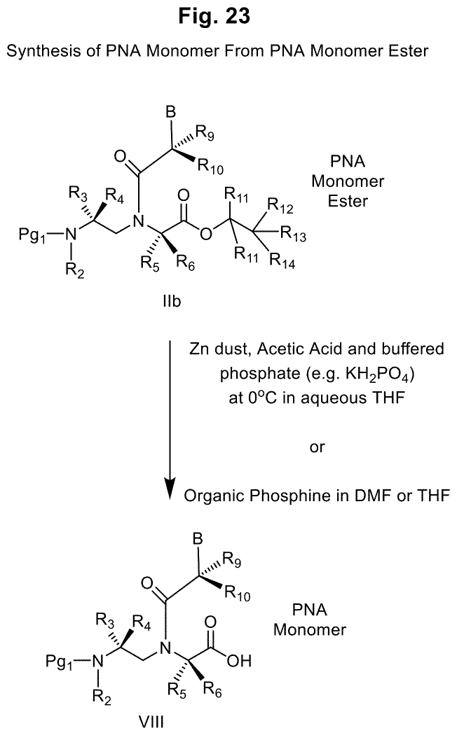

With reference to FIG. 23, Applicants have found at least two routes to very selective cleavage of the ester group of compounds of formula II or IIb. In one embodiment, zinc (in dust or fine particulate form) is combined with acetic acid and monobasic potassium phosphate in an aqueous THF mixture. This reaction is preferably carried out at 0° C. and is often completed in 2 to 24 hours depending on the nature of the ester (See: Example 11). These reducing conditions are relatively mild as determined by retention of most of the triple bond in Compound 30-10.

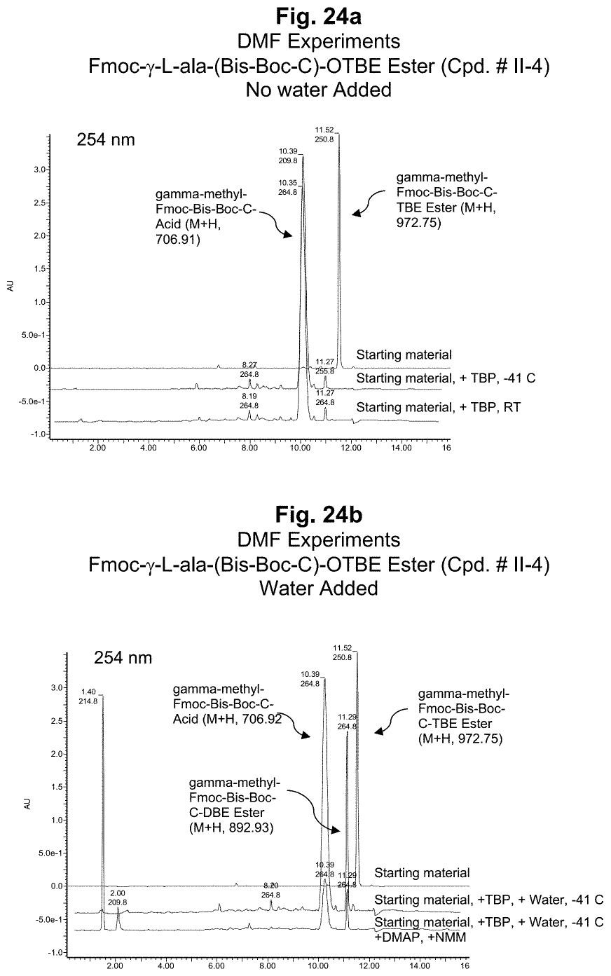

Alternatively, in some embodiments, the PNA Monomer Ester can be treated with an organophosphine reagent, optionally DMAP and an organic base (such as NMM) in an aprotic solvent such as THF or DMF (See: Examples 12 & 13). FIGS. 24a, 24b, 25, 26a and 26b are chromatograms generated using a LC/MS instrument and demonstrate success of this approach.

X. Alternative Routes to Backbone Esters and Backbone Ester Acid Salts

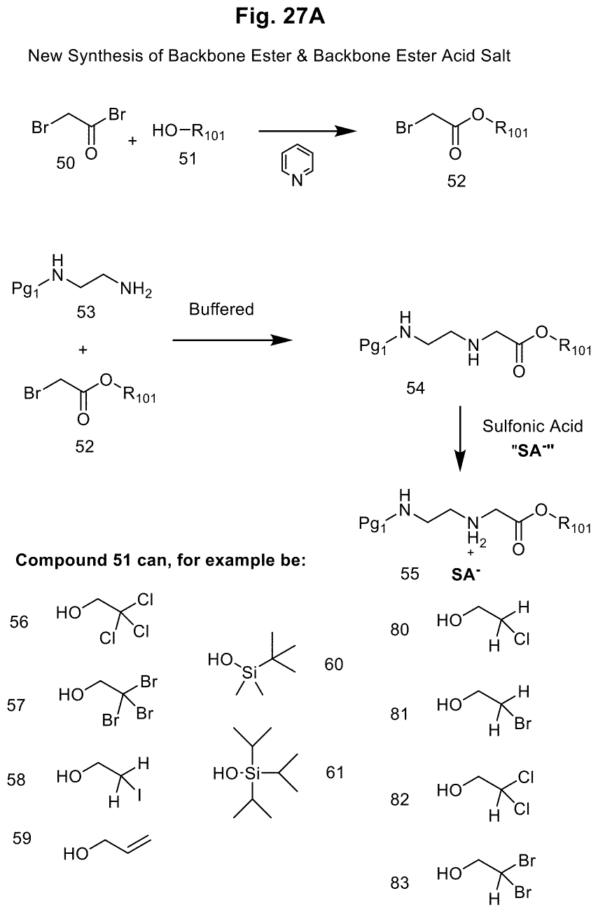

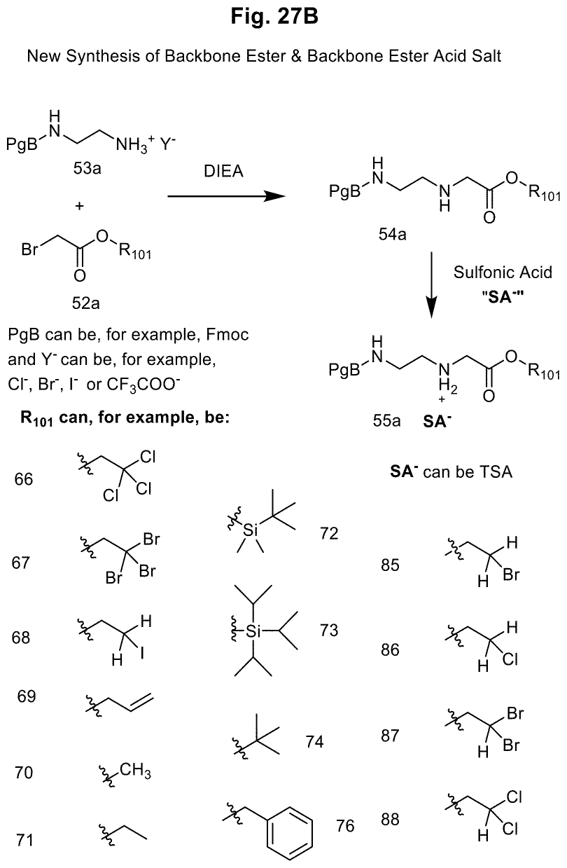

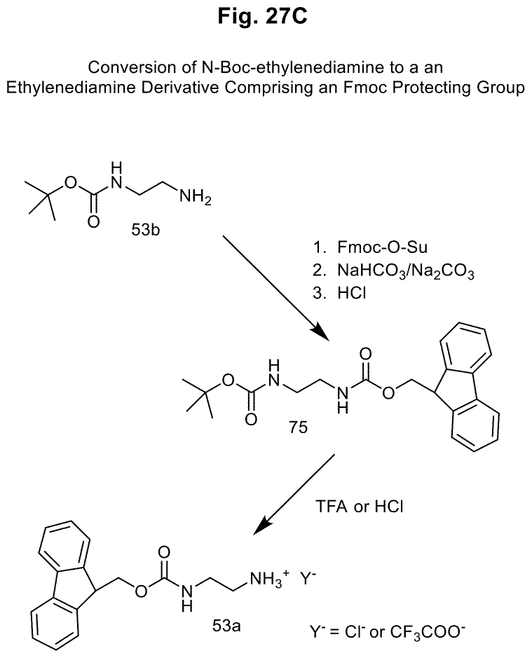

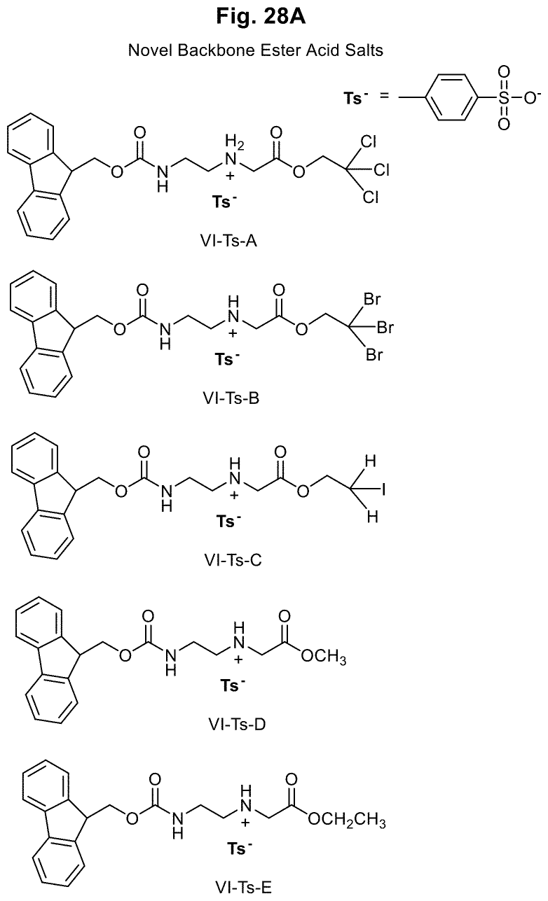

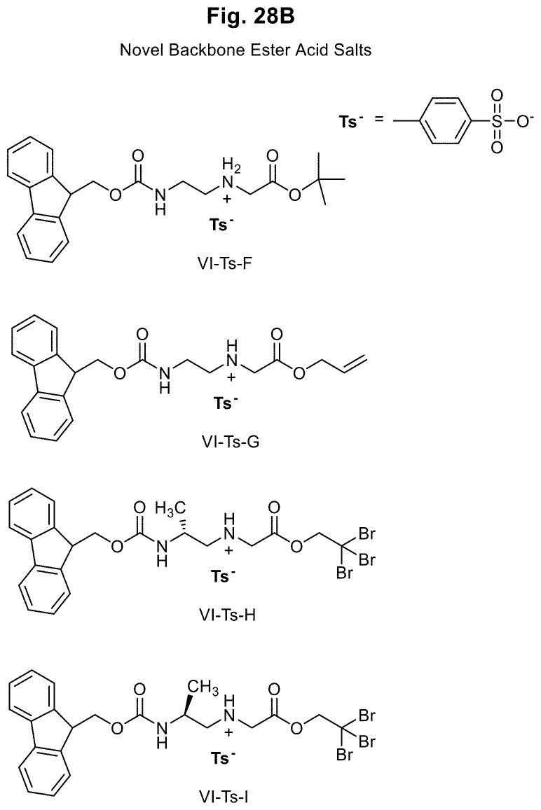

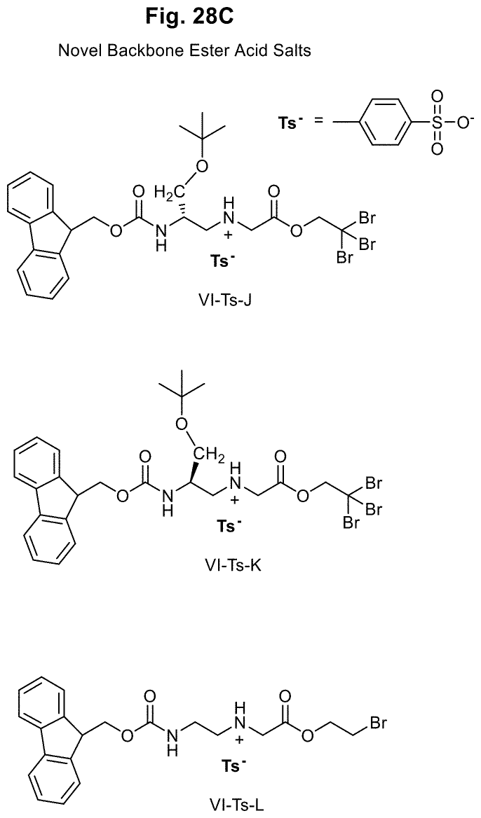

Applicants examined alternative routes to the Backbone Esters with the hope of improving the process. With reference to FIGS. 27A and 27B, an alternative synthetic route to the Backbone Esters and Backbone Ester Acid Salts is illustrated.

Numerous bromoacetate esters are commercially available. For example, many vendors sell methyl bromoacetate, ethyl bromoacetate, tert-butyl bromoacetate and/or benzyl bromoacetate. Numerous others are also commercially available or can be made as a custom synthesis. If, however, a desired bromoacetate ester is not commercially available, with reference to FIG. 27A, it is possible to react, for example, (compound 50) bromoacetyl bromide (or an equivalent reagent such as chloroacetyl chloride, bromoacetyl chloride, iodoacetyl bromide, iodoacetyl iodide or iodoacetyl chloride) with a corresponding alcohol (compound 51) that is selected based on the ester type desired. For example, if a trichloroethyl ester, tribromoethyl ester, 2-bromoethyl or 2-iodoethyl ester is desired, the selected alcohol would be 2,2,2-trichloroethanol (56), 2,2,2-tribromoethanol (57), 2-bromoethanol (81) or 2-iodoethanol (58), respectively. Some other non-limiting examples of alcohols include, allyl alcohol (59), tert-butyldimethylsilyl alcohol (60), triisopropylsilyl alcohol (61), 2-chloroethanol (80), 2,2-chloroethanol (82), 2-bromoethanol (81) and 2,2-dibromoethanol (83). In some embodiments, the alcohol is selected from 2,2,2-trichloroethanol (56), 2,2,2-tribromoethanol (57) and 2-iodoethanol (58). In some embodiments, the alcohol is selected from 2-chloroethanol (80) or 2-bromoethanol (81). In some embodiments, the alcohol is selected from 2,2-dichloroethanol (82) and 2,2-dibromoethanol (83).

The reaction can be carried out using pyridine (or collidine) as a base in an ether-based solvent such as diethyl ether, tetrahydrofuran or 1,4-dioxane, preferably obtained in dry (anhydrous) form. The reaction is preferably performed under dry/anhydrous conditions. The product of the reaction is the desired bromoacetic acid ester (compound 52). For example, compound 52 could be 2-chloroethyl bromoacetate, 2,2-dichloroethyl bromoacetate, 2,2,2-trichloroethyl bromoacetate, 2-bromoethyl bromoacetate, 2,2-dibromoethyl bromoacetate, 2,2,2-tribromoethyl bromoacetate, 2-iodoethyl bromoacetate, 2-bromoethyl bromoacetate, allyl bromoacetate, triisopropylsilyl bromoacetate, or tert-butyldimethylsilyl bromoacetate. Generally, the crude reaction product can be extracted and the crude product purified by vacuum distillation or column chromatography.

Again, with reference to FIG. 27A, the purchased or prepared bromoacetic acid esters (compound 52) can be reacted with monoprotected ethylene diamine (compound 53) in a buffered reaction to produce the Backbone Ester compound (compound 54). The reaction is buffered to minimize bis-alkylation of the amine. The reaction is preferably buffered but may contain an excess of the tertiary amine so it is basic. A similar alkylation reaction has been reported by Feagin et al., (Ref, C-31) but only using mono-Boc protected ethylenediamine. Feagin et al. did not perform the reaction with N-Fmoc-protected ethylene diamine despite ultimately producing the Fmoc-protected aminoethylglycine backbone. This illustrates a concern that performing the alkylation under basic conditions with a base-labile protecting group such as Fmoc is not expected to be successful.

The monoprotected ethylene diamine (compound 53) can in some cases be purchased. For example, N-Boc-ethylene diamine is commercially available. Ethylene diamine can be monoprotected with other protecting groups, for example, with Dmbhoc by using the process described in U.S. Pat. No. 6,063,569 (See for example FIG. 1 and Example 2 of U.S. Pat. No. 6,063,569). This procedure is particularly useful for acid-labile protecting groups.

Mono Fmoc protected ethylene diamine as its acid salt (and ethylene diamine monoprotected with other base-labile protecting groups) can be prepared from N-Boc-ethylene diamine as illustrated in FIG. 27C. As illustrated, N-Boc-ethylene diamine (53b) is reacted with Fmoc-O-Su (defined below) in a solution containing a mixture of sodium bicarbonate and sodium carbonate. This reaction can be performed in a mixture of water and an organic solvent such as acetone or acetonitrile. The mixture of sodium bicarbonate and sodium carbonate buffers the solution to permit the reaction of the free amine with the Fmoc-O-Su. When the reaction is completed, all of the sodium carbonate and bicarbonate can be neutralized with an equivalent of a strong acid (such as HCl) to give the mono Fmoc—mono Boc protected ethylene diamine (compound 75). Treatment of compound 75 with an excess of strong acid such as for example HCl or TFA will remove the Boc protecting group and produce the acid salt of the Fmoc (or other base-labile mono protected) ethylene diamine (compound 53a).

With reference to FIG. 27B, mono Boc-ethylene diamine (compound 53—FIG. 27A), a version of monoprotected ethylene diamine comprising a base-labile protecting group (compound 53a) can be reacted with a bromoacetic acid ester (52a—wherein R₁₀₁ is defined below) in the presence of a tertiary base such as DI EA (or TEA or NMM, etc.) to thereby produce the Backbone Ester (54a). In some embodiments, PgB can be Fmoc. In some embodiments, PgB can be selected from the group consisting of: Nsc, Bsmoc, Nsmoc, ivDde, Fmoc*, Fmoc(2F), mio-Fmoc, dio-Fmoc, TCP, Pms, Esc, Sps and Cyoc.

As illustrated in FIG. 27A and FIG. 27B, the Backbone Esters (54 and 54a) can be converted to their sulfonic acid salts by treatment with a sulfonic acid. Sulfonic acids include, without limitation, benzenesulfonic acid, naphthalenesulfonic acid, p-xylene-2-sulfonic acid, 2,4,5-trichlorobenzenesulfonic acid, 2,6-dimethylbenzenesulfonic acid, 2-mesitylenesulfonic acid (or dihydrate), 2-methylbenzene sulfonic acid, 2-ethylbenzenesulfonic acid, 2-isopropylbenzenesulfonic acid, 2,3-dimethylbenzenesulfonic acid, 2,4,6-trimethylbenzenesulfonic acid and 2,4,6-triisopropylbenzenesulfonic acid. Applicants have found that p-toluenesulfonic acid (TSA) is particularly useful and Backbone Ester Acid Salts of this type tend to crystallize in high purity from ethyl acetate or mixtures of ethyl acetate, ether and/or hexanes. Generally, the sulfonic acid can be added to the Backbone Ester prior to or after a purification step (e.g. column chromatography), whereinafter, the salt product will crystallize from the solution.

As mentioned above, Feagin et al., (Ref, C-31) did not react any N-protected ethylenediamine moiety with a bromoacetate where the N-protecting group was a base-labile protecting group. Indeed, it might be expected that the basic conditions needed to accommodate such an alkylation reaction would lead to such a plethora of side reactions, such that it would be impossible to isolate a product or at least not lead to a very good yield. For example, it might be expected that the basic conditions would result in significant loss of the base-labile Fmoc group. It also might be expected that the secondary amine in the backbone will bis-alkylate. It also might be expected that the secondary amine in the backbone could attach the ester group of the backbone. These types of reactions are all possible and it is known that as their free-secondary amines, these backbone moieties are not stable for long periods of time. Nevertheless, Applicants have determined that this reaction can be performed under conditions wherein the reaction proceeds in reasonable purity, such that it is possible to obtain pure products in the range of about 40-60% yield as their sulfonic acid salts. Thus, in some embodiments, this invention pertains to a simplified process for preparing compounds of the general formula 54a:

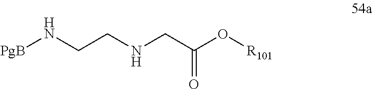

wherein, PgB is a base-labile amine protecting group (for example, Fmoc, Nsc, Bsmoc, Nsmoc, ivDde, Fmoc*, Fmoc(2F), mio-Fmoc, dio-Fmoc, TCP, Pms, Esc, Sps or Cyoc), R₁₀₁ can be a branched or straight chain C₁-C₄ alkyl group or a group of formula I;

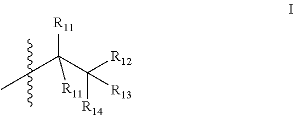

wherein, each R₁₁ can be independently H, D, F, C₁-C₆ alkyl, C₃-C₆ cycloalkyl or aryl;
each of R₁₂, R₁₃ and R₁₄ can be independently selected from H, D, F, Cl, Br and I, provided however that at least one of R₁₂, R₁₃ and R₁₄ is selected from Cl, Br and I. In some embodiments, R₁₀₁ can be a moiety selected from the group consisting of: methyl (70), ethyl (71), tert-butyl (74), benzyl (76), 2-chloroethyl (86), 2,2-dichloroethyl (88), 2,2,2-trichloroethyl (66), 2-bromoethyl (85), 2,2-dibromoethyl (87), 2,2,2-tribromoethyl (67), 2-iodoethyl (68), allyl (69), triisopropylsilyl (73), and tert-butyldimethylsilyl (72) and SA⁻ is a sulfonic acid anion. In some embodiments, R₁₀₁ is selected from 2,2,2-trichloroethyl (66), 2-bromoethyl (85), 2,2,2-tribromoethyl (67) and 2-iodoethyl (68). In some embodiments, PgB is Fmoc. In some embodiments, PgB is Fmoc and R₁₀₁ is selected from 2,2,2-trichloroethyl (66), 2-bromoethyl (85), 2,2,2-tribromoethyl (67) and 2-iodoethyl (68).

According to the method, a compound of general formula 53a:

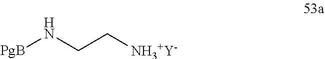

is reacted with a compound of general formula 52a:

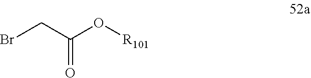

wherein, PgB, and R₁₀₁ are previously defined. The anion Y⁻ can be any anion. For example, the anion Y⁻ can be I⁻, Br⁻, Cl⁻, AcO⁻ (acetate), CF₃COO⁻ (trifluoroacetate), citrate or tosylate. The reaction can proceed in the presence of a tertiary base such as DIEA, TEA or NMM but where the equivalents are carefully controlled such that the reaction is buffered to avoid excessive decomposition. Suitable conditions are illustrated in Example 18. The reaction can be carried out in a dry/anhydrous solvent such as diethyl ether, 1,4-dioxane, tetrahydrofuran, or acetonitrile. This process eliminates the two additional steps need to remove the acid labile protecting group (i.e. Boc) from the Backbone Ester and replace it with a base-labile protecting group (as was done by Feagin et al., (Ref, C-31).

In some embodiments, the product of formula 54a:

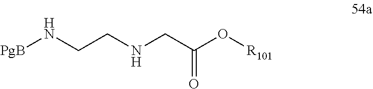

can be converted to sulfonic acid salt by treatment with a sulfonic acid to thereby produce a compound of formula 55a:

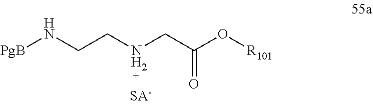

wherein, PgB, R₁₀₁ and SA⁻ are as previously defined.

This novel process is very well suited for the production of Backbone Esters and Backbone Ester Acid Salts that can be used for producing classic PNA monomers (i.e. monomers having a N-Fmoc-2-(aminoethyl)glycine backbone). With available substituted chiral amines, this procedure could be extended to produce backbones comprising a β- or γ-backbone modification. Similarly, with available chiral substituted bromoacetates, this procedure could be extended to produce backbones comprising an α-backbone modification.

XI. Advantages

It is an advantage of the sulfonic acid salts of the Backbone Esters of the present invention that they are generally stable, highly crystalline, and can be recrystallized. Accordingly, the Backbone Ester Acid Salts (as their sulfonic acid salts) can, in some cases, be prepared without column purification of the crude Backbone Ester.

Applicants have demonstrated that the PNA Monomers produced by removal of the 2,2,2-tribromoethyl protecting group and 2-iodoethyl protecting group of a PNA Monomer Ester can generally produce PNA oligomers of higher purity than PNA oligomers produced from commercially available PNA monomers having comparable purity specifications, but with different impurity profiles (data not shown). Furthermore, additional data has shown that because the impurity profiles of commercially available PNA monomers differ from those produced by this process, for PNA monomers of comparable purity specifications (i.e. their percent purity as determined HPLC analysis at 260 m), PNA monomers produced by this process often produce higher quality PNA oligomers PNA oligomers of higher purity based on HPLC analysis under identical conditions when analyzed at 260 nm)

The process described herein for preparing compounds of formulas 54 and 54a significantly reduces the steps involved in preparation of the Backbone Ester comprising a base-labile protecting group as compared with, for example, Feagin et al., (Ref, C-31). Furthermore, this process uses inexpensive and readily available starting materials.

### 5. Various Embodiments of the Invention

With respect to this section 5 and the claims, it should be understood that the order of steps or order for performing certain actions is immaterial so long as the present teachings remain operable or unless otherwise specified. Moreover, in some embodiments, two or more steps or actions can be conducted simultaneously so long as the present teachings remain operable or unless otherwise specified.

I. Backbone Ester Acid Salts

As noted above, in some embodiments, the Backbone Ester can be converted to a Backbone Ester Acid Salt by treatment of the Backbone Ester with an appropriate acid. Therefore, in some embodiments, this invention pertains to a compound (e.g., an organic salt) compound of formula VI:

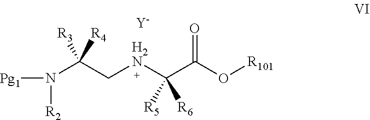

wherein: Y⁻ is a sulfate or sulfonate anion (e.g. tosylate); Pg₁ is an amine protecting group; R₁₀₁ is a branched or straight chain C₁-C₄ alkyl group or a group of formula I;

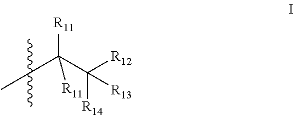

wherein, each R₁₁ is independently H, D, F, C₁-C₆ alkyl, C₃-C₆ cycloalkyl or aryl; each of R₁₂, R₁₃ and R₁₄ is independently selected from the group consisting of: H, D, F, Cl, Br and I, provided however that at least one of R₁₂, R₁₃ and R₁₄ is selected from Cl, Br and I. With respect to formula VI, R₂ can be H, D or C₁-C₄ alkyl; each of R₃, R₄, R₅, and R₆ can be independently selected from the group consisting of: H, D, F, and a side chain selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIy, IIIw, IIIx, IIIy, IIIz, IIIaa, and IIIab, wherein each of IIIi, IIIj, IIIk, IIIm, IIIn, IIIo, IIIp, IIIq, IIIr, IIIs, IIIt, IIIu, IIIv, IIIw, IIIx, IIIy and IIIz optionally comprises a protecting group (See Section 4(VII), above, for a discussion of various amino acid side chain protecting groups);

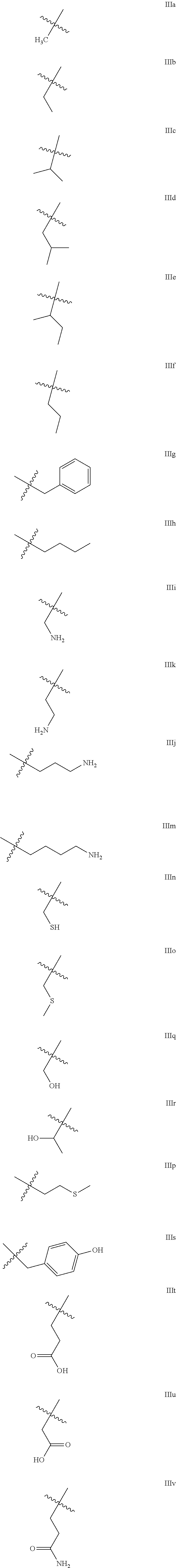

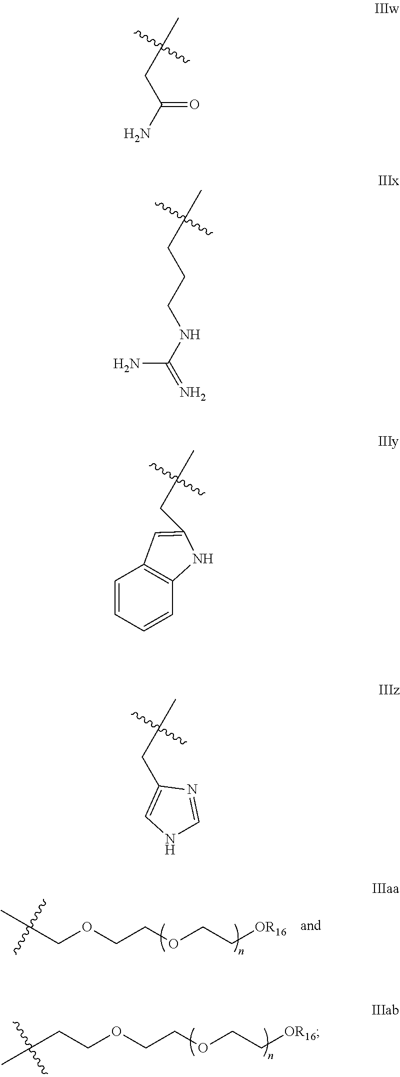

wherein, R₁₆ can be selected from H, D and C₁-C₄ alkyl group; and n can be a number from 0 to 10, inclusive.

In some embodiments of formula VI, the sulfate or sulfonate anion is produced from an acid selected from the group consisting of: benzenesulfonic acid, naphthalenesulfonic acid, p-xylene-2-sulfonic acid, 2,4,5-trichlorobenzenesulfonic acid, 2,6-dimethylbenzenesulfonic acid, 2-mesitylenesulfonic acid (or dihydrate), 2-methylbenzene sulfonic acid, 2-ethylbenzenesulfonic acid, 2-isopropylbenzenesulfonic acid, 2,3-dimethylbenzenesulfonic acid, 2,4,6-trimethylbenzenesulfonic acid and 2,4,6-triisopropylbenzenesulfonic acid. In some embodiments of formula VI, the sulfate or sulfonate anion is produced from an acid selected from the group consisting of: benzenesulfonic acid, naphthalenesulfonic acid, p-xylene-2-sulfonic acid, 2,4,5-trichlorobenzenesulfonic acid, 2,6-dimethylbenzenesulfonic acid, 2-mesitylenesulfonic acid (or dihydrate), 2-methylbenzene sulfonic acid, 2-ethylbenzenesulfonic acid, 2-isopropylbenzenesulfonic acid, 2,3-dimethylbenzenesulfonic acid, and 2,4,6-triisopropylbenzenesulfonic acid.

In some embodiments, the anion is produced from p-toluenesulfonic acid. The sulfate or sulfonate anion is produced because upon reaction with the secondary amine of the Backbone Ester, the secondary amine is protonated by the acidic proton of the acid, thereby producing the sulfate or sulfonate anion.

In some embodiments of formula VI, Y⁻ is selected from benzenesulfonate, p-toluenesulfonate, naphthalenesulfonate, p-xylene-2-sulfonate, 2,4,5-trichlorobenzenesulfonate, 2,6-dimethylbenzenesulfonate, 2-mesitylenesulfonate, 2-mesitylenesulfonate dihydrate, 2-methylbenzene sulfonate, 2-ethylbenzenesulfonate, 2-isopropylbenzenesulfonate, 2,3-dimethylbenzenesulfonate, 2,4,6-trimethylbenzenesulfonate, and 2,4,6-triisopropylbenzenesulfonate. In some embodiments, Y− is p-toluenesulfonate.

In some embodiments of formula VI, Y⁻ is benzenesulfonate. In some embodiments, Y− is

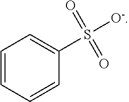

In some embodiments, Y⁻ is p-toluenesulfonate. In some embodiments, Y⁻ is

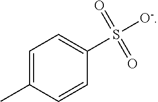

In some embodiments, Y⁻ is naphthalenesulfonate. In some embodiments, Y⁻ is

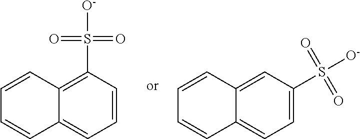 [SMILES: O=S(=O)([O-])c1cccc2ccccc12 ; O=S(=O)([O-])c1ccc2ccccc2c1]

In some embodiments, Y⁻ is

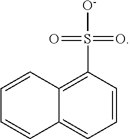 [SMILES: O=S(=O)([O-])c1cccc2ccccc12]

In some embodiments, Y⁻ is

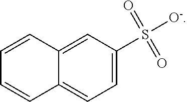

In some embodiments, Y⁻ is p-xylene-2-sulfonate.

In some embodiments, Y⁻ is

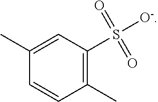

In some embodiments, Y⁻ is 2,4,5-trichlorobenzenesulfonate.

In some embodiments, Y⁻ is

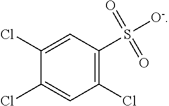

In some emboidments, Y⁻ is 2,6-dimethylbenzenesulfonate.

In some emboidments, Y⁻ is

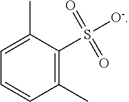

In some emboidments, Y⁻ is 2-mesitylenesulfonate.

In some emboidments, Y⁻ is

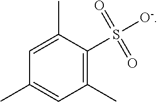

In some emboidments, Y⁻ is 2-mesitylenesulfonate dihydrate.

In some emboidments, Y⁻ is

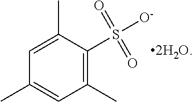

In some emboidments, Y⁻ is 2-methylbenzene sulfonate.

In some emboidments, Y⁻ is

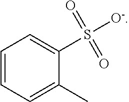

In some emboidments, Y⁻ is 2-ethylbenzenesulfonate.

In some embodiments, Y⁻ is.

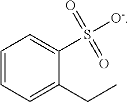

In some embodiments, Y⁻ is 2-isopropylbenzenesulfonate.

In some embodiments, Y⁻ is

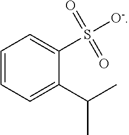

In some embodiments, Y⁻ is 2,3-dimethylbenzenesulfonate.

In some embodiments, Y⁻ is

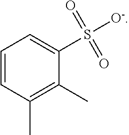

In some embodiments, Y⁻ is 2,4,6-triisopropylbenzenesulfonate.

In some embodiments, Y⁻ is

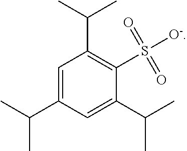

In some embodiments of formula VI, at least one of R₃ and R₄ can be the group of formula IIIaa. In some embodiments, R₁₆ can be selected from the group consisting of: H, D, methyl and t-butyl and n is selected from 1, 2, 3 and 4.

In some embodiments of formula VI, R₂ is H or D. In some embodiments, R₁₆ is selected from the group consisting of: H, D, methyl and t-butyl, and n is 1, 2, 3 or 4. In some embodiments, R₂ is H, R₁₆ is methyl or t-butyl, and n is 1 or 2.

In some embodiments of formula VI, R₁₆ is selected from the group consisting of: H, D, methyl, ethyl and t-butyl, and n is 1, 2, 3 or 4. In some embodiments, R₂ is H or CHs, R₁₆ is methyl or t-butyl, and n is 1, 2 or 3.

In some embodiments of formula VI, each of R₅ and R₆ is independently: H, D or F.

In some embodiments of formula VI, Pg₁ is selected from the group consisting of: Nsc, Bsmoc, Nsmoc, ivDde, Fmoc*, Fmoc(2F), mio-Fmoc, dio-Fmoc, TCP, Pms, Esc, Sps and Cyoc. In some embodiments of formula VI, Pg₁ is Fmoc.

In some embodiments of formula VI, Pg₁ is selected from the group consisting of: Trt, Ddz, Bpoc, Nps, Bhoc, Dmbhoc and Floc. In some embodiments of formula VI, Pg₁ is Boc.

In some embodiments of formula VI, R₁₀₁ is selected from the group consisting of: methyl, ethyl, tert-butyl, allyl, 2-iodoethyl, 2-bromoethyl, 2,2,2-trifluoroethyl, 2,2,2-trichloroethyl, 2,2,2-tribromoethyl and tert-butyldimethylsilyl. In some embodiments of formula VI, R₁₀₁ is selected from the group consisting of: 2-iodoethyl, 2-bromoethyl, 2,2,2-trichloroethyl and 2,2,2-tribromoethyl. In some embodiments of formula VI, R₁₀₁ is selected from the group consisting of: methyl, ethyl, n-propyl, isopropyl, n-butyl, iso-butyl, sec-butyl, tert-butyl, allyl, 2-iodoethyl, 2,2,2-trichloroethyl, 2,2,2-trifluoroethyl, 2,2,2-tribromoethyl and tert-butyldimethylsilyl.

In some embodiments of formula VI, (i) one of R₃, R₄, R₅ and R₆ is independently selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIy, IIIw, IIIx, IIIy, IIIz IIIaa and IIIab, wherein each of IIIi, IIIj, IIIk, IIIm, IIIn, IIIo, IIIp, IIIq, IIIr, IIIs, IIIt, IIIu, IIIy, IIIw, IIIx, IIIy and IIIz optionally comprises a protecting group; and (ii) the others of R₃, R₄, R₅ and R₆ are independently H, D, or F. In some embodiments, each of R₅ and R₆ is independently H or D. In some embodiments, R₁₆ is H, methyl, or t-butyl, and n is 1, 2, 3 or 4. In some embodiments, R₂ is H or CH₃, R₁₆ is methyl or t-butyl, and n is 1, 2 or 3.

In some embodiments of formula VI, each of R₅ and R₆ is independently H, D or F; and (i) one of R₃ and R₄, is independently selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIv, IIIw, IIIx, IIIy, IIIz, IIIaa, and IIIab, wherein each of IIIi, IIIj, IIIk, IIIm, IIIn, IIIo, IIIp, IIIq, IIIr, IIIs, IIIt, IIIu, IIIv, IIIw, IIIx, IIIy and IIIz optionally comprises a protecting group; and (ii) the other of R₃ and R₄ is H, D or F. In some embodiments, each of R₅ and R₆ is independently H or F. In some embodiments, each of R₅ and R₆ is H. In some embodiments, each of R₅ and R₆ is independently H, D or F; R₁₆ is selected from H, methyl, and t-butyl; and n is 1, 2, 3 or 4. In some embodiments, R₂ can be H or CHs, R₁₆ can be methyl or t-butyl and n can be 1, 2 or 3.

In some embodiments of formula VI, (i) one of R₃ and R₄ is independently selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIv, IIIw, IIIx, IIIy, IIIz, IIIaa, and IIIab, wherein each of IIIi, IIIj, IIIk, IIIm, IIIn, IIIo, IIIp, IIIq, IIIr, IIIs, IIIt, IIIu, IIIv, IIIw, IIIx, IIIy and IIIz optionally comprises a protecting group; and (ii) one of R₅, and R₆ is independently selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIv, IIIw, IIIx, IIIy, IIIz, IIIaa, and IIIab, wherein each of IIIi, IIIj, IIIk, IIIm, IIIn, IIIo, IIIp, IIIq, IIIr, IIIs, IIIt, IIIu, IIIv, IIIw, IIIx, IIIy and IIIz optionally comprises a protecting group; (iii) the other of R₃ and R₄ is H, D or F; and (iv) the other of R₅ and R₆ is H, D or F. In some embodiments, R₁₆ is selected from H, methyl, and t-butyl; and n is 1, 2, 3 or 4. In some embodiments, R₂ can be H or CHs, R₁₆ can be methyl or t-butyl and n can be 1, 2 or 3.

In some embodiments of formula VI, (i) one of R₃ and R₄, is a group of formula IIIaa; and (ii) the other of R₃ and R₄ is H or D; each of R₅ and R₆ is independently H, D, or F, R₁₆ is selected from H, methyl, and t-butyl; and n is 1, 2, 3 or 4. In some embodiments, R₂ can be H or CHs, R₁₆ can be methyl or t-butyl and n can be 1, 2 or 3.

In some embodiments of formula VI, each of R₃ and R₄ is independently H or D.

In some embodiments of formula VI, each of R₅ and R₆ is independently H or D.

In some embodiments of formula VI, one of R₃ or R₄ is a group of formula IIIaa:

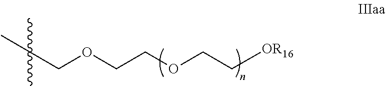

and the other of R₃ and R₄ is H, wherein, n is 0, 1, 2 or 3 and R₁₆ is H, methyl or t-butyl.

In some embodiments of formula VI, one of R₃ or R₄ is a group of formula IIIaa:

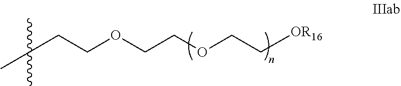

and the other of R₃ and R₄ is H, wherein, n is 0, 1, 2 or 3 and R₁₆ is H, methyl or t-butyl.

In some embodiments of formula VI, Pg₁ is a base-labile protecting group selected from the group consisting of: Fmoc, Nsc, Bsmoc, Nsmoc, ivDde, Fmoc*, Fmoc(2F), mio-Fmoc, dio-Fmoc, TCP, Pms, Esc, Sps and Cyoc. In some embodiments of formula VI, Pg₁ is a base-labile protecting group selected from the group consisting of: Fmoc, Nsc, Bsmoc, Nsmoc, Fmoc*, Fmoc(2F), mio-Fmoc, dio-Fmoc, Pms, and Cyoc. In some embodiments of formula VI, Pg₁ is Fmoc or Bsmoc. In some embodiments of formula VI, Pg₁ is Fmoc.

In some embodiments of formula VI, Pg₁ is an acid-labile protecting group selected from the group consisting of: Boc, Trt, Ddz, Bpoc, Nps, Bhoc, Dmbhoc and Floc. In some embodiments of formula VI, Pg₁ is an acid-labile protecting group selected the group consisting of: Boc, Trt, Bhoc and Dmbhoc. In some embodiments of formula VI, Pg₁ is Boc or Trt. In some embodiments of formula VI, Pg₁ is Boc. In some embodiments of formula VI, Pg₁ is Dmbhoc.

In some embodiments of formula VI, R₁₀₁ is selected from 2,2,2-trichloroethyl (TCE), 2,2,2-tribromoethyl (TBE), 2-iodoethyl (2-IE) or 2-bromoethyl (2-BrE). In some embodiments of formula VI, R₁₀₁ is 2,2,2-trichloroethyl (TCE) or 2,2,2-tribromoethyl (TBE). In some embodiments of formula VI, R₁₀₁ is 2,2,2-tribromoethyl (TBE). In some embodiments of formula VI, R₁₀₁ is 2-iodoethyl (2-IE). In some embodiments of formula VI, R₁₀₁ is 2-bromoethyl (2-BrE).

In some embodiments, the compound of formula VI is a compound of formula VI-T:

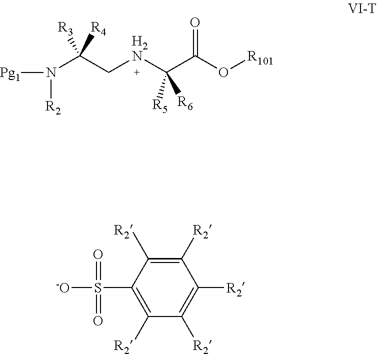

wherein, Pg₁ can be an amine protecting group; Him can be selected from the group consisting of: methyl, ethyl, n-propyl, isopropyl, n-butyl, iso-butyl, sec-butyl, tert-butyl, allyl, 2-iodoethyl, 2-bromoethyl, 2,2,2-trifluoroethyl, 2,2,2-trichloroethyl, 2,2,2-tribromoethyl and tert-butyldimethylsilyl; R₂ can be H, D or C₁-C₄ alkyl; each R₂′ is independently H, D, F, Cl, Br, I or C₁-C₄ alkyl; and each of R₃, R₄, R₅, and R₆ can be independently selected from the group consisting of: H, D, F, and a side chain selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIv, IIIw and IIIaa, wherein each of IIIi, IIIj, IIIk, IIIm, IIIn, IIIo, IIIp, IIIq, IIIr, IIIs, IIIt, IIIu, IIIv, IIIw, IIIx, IIIy and IIIz optionally comprises a protecting group;

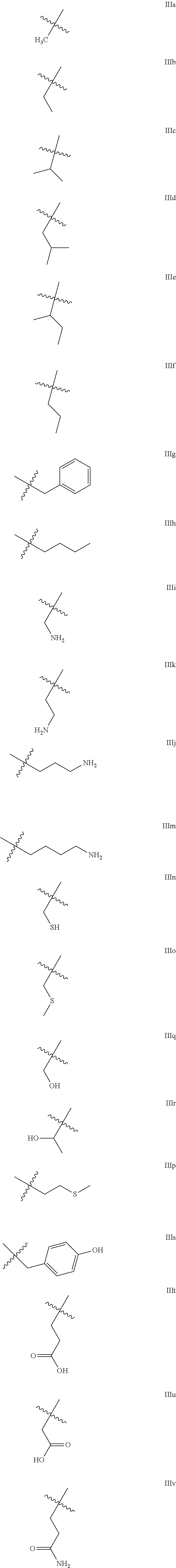

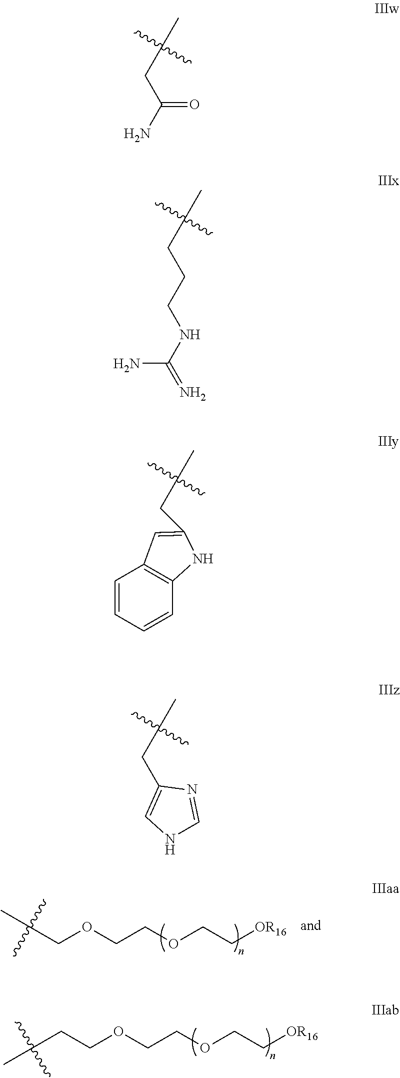

wherein, R₁₆ can be selected from H, D and C₁-C₄ alkyl group; and n can be a number from 0 to 10, inclusive. In some embodiments of compounds of formula VI-T; the sulfonate anion is produced from p-toluenesulfonic acid.

Therefore, in some embodiments, this invention pertains to a compound (e.g., an organic salt compound) of formula VI-Ts:

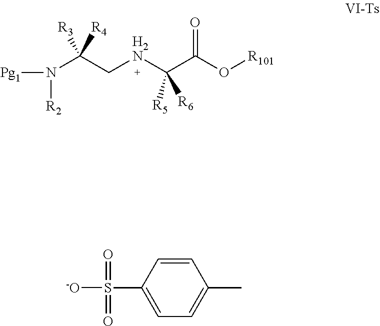

wherein, Pg₁ can be an amine protecting group; R₁₀₁ can be selected from the group consisting of: methyl, ethyl, n-propyl, isopropyl, n-butyl, iso-butyl, sec-butyl, tert-butyl, allyl, 2-iodoethyl, 2-bromoethyl, 2,2,2-trifluoroethyl, 2,2,2-trichloroethyl, 2,2,2-tribromoethyl and tert-butyldimethylsilyl; R₂ can be H, D or C₁-C₄ alkyl; and each of R₃, R₄, R₅, and R₆ is independently selected from the group consisting of: H, D, F, and a side chain selected from the group consisting of: IIIa, IIIb, IIIc, IIId, IIIe, IIIf, IIIg, IIIh, IIIi, IIIj, IIIk, IIIm, IIIn, IIIo, IIIp, IIIg, IIIr, IIIs, IIIt, IIIu, IIIy, IIIw, IIIx, IIIy, IIIz, IIIaa, and IIIab, wherein each of IIIi, IIIj, IIIk, IIIm, IIIn, IIIo, IIIp, IIIg, IIIr, IIIs, IIIt, IIIu, IIIv, IIIw, IIIx, IIIy and IIIz optionally comprises a protecting group;

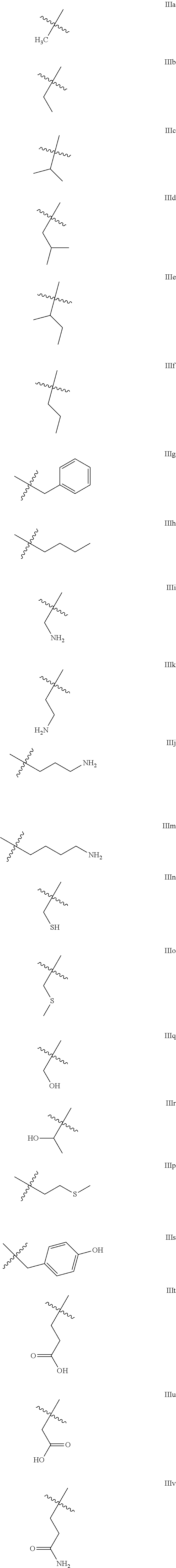

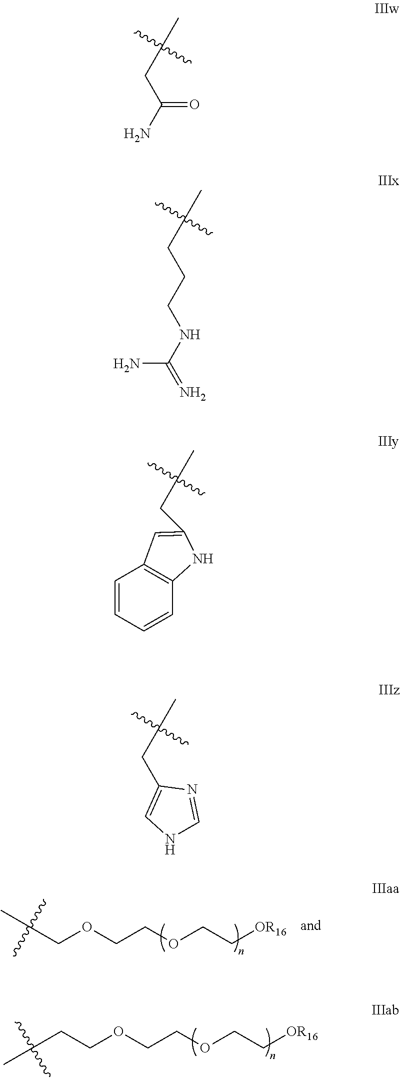

wherein, R₁₆ can be selected from H, D and C₁-C₄ alkyl group; and n can be a number from 0 to 10, inclusive.

In some embodiments of compounds of formula VI-T or VI-Ts, at least one of R₃ and R₄ can be the group of formula IIIaa. In some embodiments of compounds of formula VI-T or VI-Ts, at least one of R₃ and R₄ can be the group of formula IIIab. In some embodiments, R₁₆ can be selected from the group consisting of: H, D, methyl and t-butyl, and n can be 1, 2, 3 or 4. In some embodiments, R₂ can be H or D. In some embodiments, R₂ can be H, R₁₆ can be methyl or t-butyl, and n can be 1 or 2. In some embodiments, each of R₅ and R₆ can be independently: H, D or F.

In some embodiments of compounds of formula VI-T or VI-Ts, R₁₆ can be selected from the group consisting of: H, D, methyl and t-butyl and n is selected from 1, 2, 3 and 4. In some embodiments of the foregoing compounds, R₂ can be H or D. In some embodiments of the foregoing compounds, one of R₃ or R₄ can be a group of formula IIIaa:

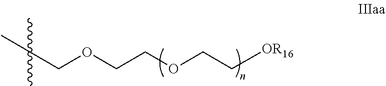

and the other of R₃ and R₄ can be H, wherein, n can be 0, 1, 2 or 3, and R₁₆ can be methyl or t-butyl.

In some embodiments of compounds of formula VI-T or VI-Ts; one of R₃, R₄, R₅ and R₆ can independently be selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIy, IIIw, IIIx, IIIy, IIIz, IIIaa, and IIIab, wherein each of IIIi, IIIj, IIIk, IIIm, IIIn, IIIo, IIIp, IIIq, IIIr, IIIs, IIIt, IIIu, IIIy, IIIw, IIIx, IIIy and IIIz optionally comprises a protecting group; and the others of R₃, R₄, R₅ and R₆ can be independently H, D or F.

In some embodiments of compounds of formula VI-T or VI-Ts, each of R₅ and R₆ can be independently H, D or F; one of R₃ and R₄ can be independently selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIv, IIIw, IIIaa, and IIIab, wherein each of IIIi, IIIj, IIIk, IIIm, IIIn, IIIo, IIIp, IIIq, IIIy, IIIs, IIIt, IIIu, IIIv, IIIw, IIIx, IIIy and IIIz optionally comprises a protecting group; and the other of R₃ and R₄ can be H, D or F.

In some embodiments of compounds of formula VI-T or VI-Ts; one of R₃ or R₄ can be a group of formula IIIaa:

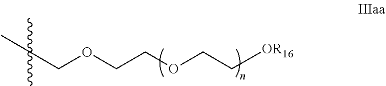

and the other of R₃ and R₄ can be H, wherein, n can be 0, 1, 2, 3 or 4 and R₁₆ can be H, methyl or t-butyl.

In some embodiments of compounds of formula VI-T or VI-Ts; one of R₃ or R₄ can be a group of formula IIIab:

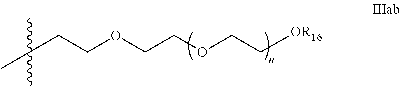

and the other of R₃ and R₄ can be H, wherein, n can be 0, 1, 2, 3 or 4 and R₁₆ can be H, methyl or t-butyl.

In some embodiments of compounds of formula VI-T or VI-Ts; Pg₁ can be selected from the group consisting of: Fmoc, Nsc, Bsmoc, Nsmoc, ivDde, Fmoc*, Fmoc(2F), mio-Fmoc, dio-Fmoc, TCP, Pms, Esc, Sps and Cyoc. In some embodiments of compounds of formula VI-T or VI-Ts; Pg₁ can be selected from the group consisting of: Fmoc, and Bsmoc. In some embodiments of compounds of formula VI-T or VI-Ts; Pg₁ can be Fmoc.

In some embodiments of compounds of formula VI-T or VI-Ts; Pg₁ can be selected from the group consisting of: Boc, Trt, Ddz, Bpoc, Nps, Bhoc, Dmbhoc and Floc. In some embodiments of compounds of formula VI-T or VI-Ts; Pg₁ can be selected from the group consisting of: Boc, Dmbhoc and Fmoc. In some embodiments of compounds of formula VI-T or VI-Ts; Pg₁ can be Boc.

In some embodiments of any of the compounds based on formulas VI-T and VI-Ts, each R₃, R₄, R₅ and R₆ can be independently H, D or F. In some embodiments of any of the compounds based on formulas VI-T and VI-Ts, Pg₁ can be Fmoc and each of R₃, R₄, R₅ and R₆ can be H. In some embodiments of any of the compounds based on formulas VI-T and VI-Ts, one of R₃ and R₄ can be methyl and the other or R₃ and R₄ can be H and R₅ and R₆ can be H.

In some embodiments of compounds of formula VI-T or VI-Ts; R₁₀₁ can be methyl, ethyl, tert-butyl, allyl, 2-iodoethyl, 2-bromoethyl, 2,2,2-trifluoroethyl, 2,2,2-trichloroethyl, 2,2,2-tribromoethyl and tertbutyldimethylsilyl. In some embodiments of compounds of formula VI-T or VI-Ts; R₁₀₁ can be methyl, ethyl, n-propyl, isopropyl, n-butyl, iso-butyl, sec-butyl, tert-butyl, allyl, 2-iodoethyl, 2-bromoethyl, 2,2,2-trifluoroethyl, 2,2,2-trichloroethyl, 2,2,2-tribromoethyl and tert-butyldimethylsilyl. In some embodiments of compounds of formula VI-T or VI-Ts; R₁₀₁ can be group of formula I;

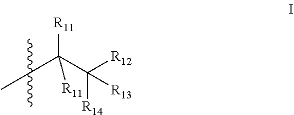

wherein, each R₁₁ can be H, D, F, C₁-C₆ alkyl, C₃-C₆ cycloalkyl or aryl; and
each of R₁₂, R₁₃ and R₁₄ can independently be selected from H, D, F, Cl, Br and I, provided however that at least one of R₁₂, R₁₃ and R₁₄ is selected from Cl, Br and I. In some embodiments of compounds of formula VI-T or VI-Ts; R₁₀₁ is selected from methyl, ethyl, tert-butyl, allyl, or tert-butyldimethylsilyl. In some embodiments of compounds of formula VI-T or VI-Ts; R₁₀₁ is selected from 2,2,2-trichloroethyl, 2,2,2-tribromoethyl, 2-iodoethyl and 2-bromoethyl. In some embodiments of compounds of formula VI-T or VI-Ts; R₁₀₁ is 2,2,2-tribromoethyl.

In some embodiments of compounds of formula VI-T or VI-Ts; each R₃, R₄, R₅ and R₆ is independently H, D or F. In some embodiments of compounds of formula VI-T or VI-Ts; Pg₁ is Fmoc, R₂ is H, and each of R₃, R₄, R₅ and R₆ is H. In some embodiments of compounds of formula VI-T or VI-Ts; Pg₁ is Boc, R₂ is H, and each of R₃, R₄, R₅ and R₆ is H

In some embodiments, the compound of formula VI-Ts has the structure VI-Ts-A:

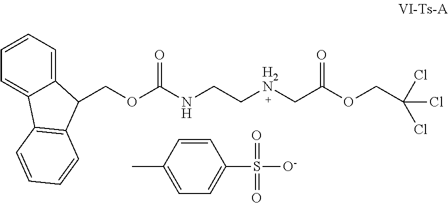 [SMILES: O=C(CNCCNC(=O)OCC1c2ccccc2-c2ccccc21)OCC(Cl)(Cl)Cl ; Cc1ccc(S(=O)(=O)[O-])cc1]

In some embodiments, the compound of formula VI-Ts has the structure VI-Ts-B:

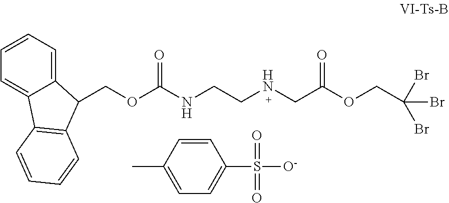 [SMILES: O=C(CNCCNC(=O)OCC1c2ccccc2-c2ccccc21)OCC(Br)(Br)Br ; Cc1ccc(S(=O)(=O)[O-])cc1]

In some embodiments, the compound of formula VI-Ts has the structure VI-Ts-C:

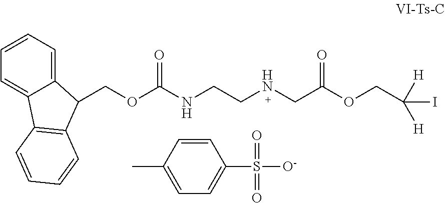 [SMILES: O=C(CNCCNC(=O)OCC1c2ccccc2-c2ccccc21)OCCI ; Cc1ccc(S(=O)(=O)[O-])cc1]

In some embodiments, the compound of formula VI-Ts has the structure VI-Ts-D:

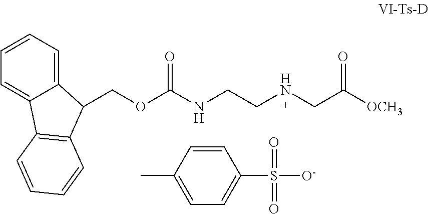 [SMILES: COC(=O)CNCCNC(=O)OCC1c2ccccc2-c2ccccc21 ; Cc1ccc(S(=O)(=O)[O-])cc1]

In some embodiments, the compound of formula VI-Ts has the structure VI-Ts-E:

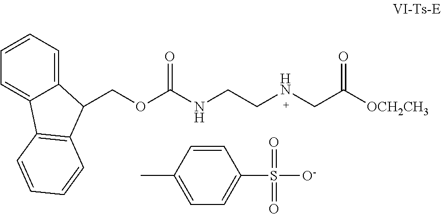 [SMILES: CCOC(=O)CNCCNC(=O)OCC1c2ccccc2-c2ccccc21 ; Cc1ccc(S(=O)(=O)[O-])cc1]

In some embodiments, the compound of formula VI-Ts has the structure VI-Ts-F:

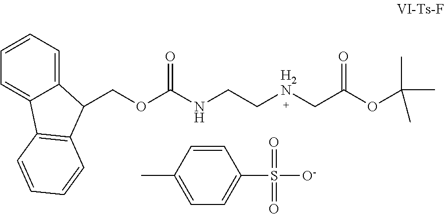 [SMILES: CC(C)(C)OC(=O)CNCCNC(=O)OCC1c2ccccc2-c2ccccc21 ; Cc1ccc(S(=O)(=O)[O-])cc1]

In some embodiments, the compound of formula VI-Ts has the structure VI-Ts-G:

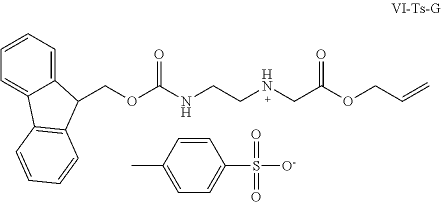 [SMILES: C=CCOC(=O)CNCCNC(=O)OCC1c2ccccc2-c2ccccc21 ; Cc1ccc(S(=O)(=O)[O-])cc1]

In some embodiments, the compound of formula VI-Ts has the structure VI-Ts-H:

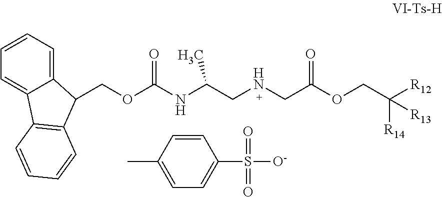

wherein, each of R₁₂, R₁₃ and R₁₄ is independently H, D, F, Cl, Br or I, provided however that at least one of R₁₂, R₁₃ and R₁₄ is selected from Cl, Br and I.

In some embodiments, the compound of formula VI-Ts has the structure VI-Ts-I:

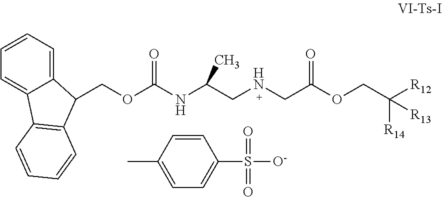

wherein, each of R₁₂, R₁₃ and R₁₄ is independently H, D, F, Cl, Br or I, provided however that at least one of R₁₂, R₁₃ and R₁₄ is selected from Cl, Br and I.

In some embodiments, the compound of formula VI-Ts has the structure VI-Ts-J:

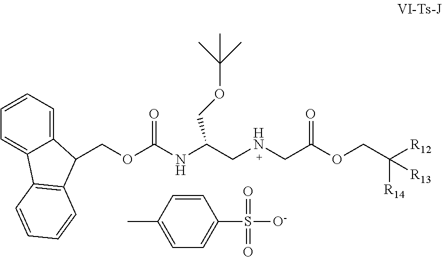

wherein, each of R₁₂, R₁₃ and R₁₄ is independently H, D, F, Cl, Br or I, provided however that at least one of R₁₂, R₁₃ and R₁₄ is selected from Cl, Br and I.

In some embodiments, the compound of formula VI-Ts has the structure VI-Ts-K:

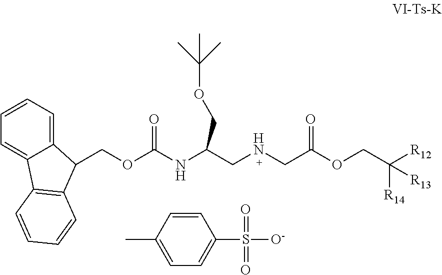

wherein, each of R₁₂, R₁₃ and R₁₄ is independently H, D, F, Cl, Br or I, provided however that at least one of R₁₂, R₁₃ and R₁₄ is selected from Cl, Br and I.

In some embodiments, the compound of formula VI-Ts has the structure VI-Ts-B:

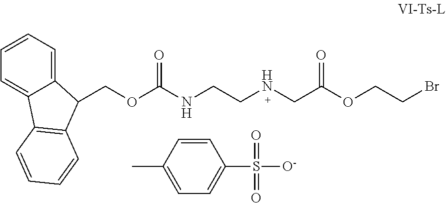 [SMILES: O=C(CNCCNC(=O)OCC1c2ccccc2-c2ccccc21)OCCBr ; Cc1ccc(S(=O)(=O)[O-])cc1]

II. Methods for Producing Backbone Esters and Backbone Ester Acid Salts

In some embodiments, this invention pertains to novel methods for producing Backbone Esters and Backbone Ester Acid Salts. For example and with reference to FIG. 27B, in some embodiments, this invention pertains to a method comprising reacting a compound of formula 53a:

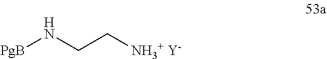

with a compound of formula 52a:

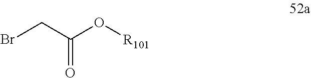

wherein PgB can be a base-labile amine protecting group; R₁₀₁ can be a branched or straight chain C₁-C₄ alkyl group or a group of formula I;

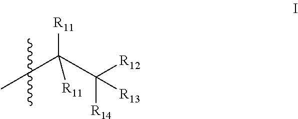

wherein, each R₁₁ can be independently H, D, F, C₁-C₆ alkyl, C₃-C₆ cycloalkyl or aryl; each of R₁₂, R₁₃ and R₁₄ can be independently selected from H, D, F, Cl, Br and I, provided however that at least one of R₁₂, R₁₃ and R₁₄ is selected from Cl, Br and I; and
Y⁻ is an anion, such as Cl−, Br−, I−, trifluoroacetate, acetate citrate and tosylate.

The alkylation reaction can proceed in the presence of a tertiary base to produce a product of formula 54a:

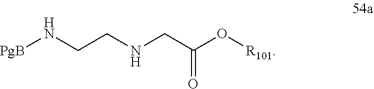

wherein, PgB and R₁₀₁ are defined above. In some embodiment, R₁₀₁ can be methyl (formula 70; See: FIG. 27B), ethyl (formula 71), tert-butyl (formula 74), benzyl (formula 76), 2,2,2-trichloroethyl (formula 66), 2,2,2-tribromoethyl (formula 67), 2-iodoethyl (formula 68), 2-bromoethyl (formula 85), allyl (formula 69), triisopropylsilyl (formula 73), or tert-butyldimethylsilyl (formula 72).

Generally, the reaction can be performed in an organic solvent such as diethyl ether, THF or 1,4-dioxane. The reaction can also proceed in a polar aprotic solvent such as acetonitrile.

In some embodiments, the method further comprises contacting the compound of formula 54a with at least one equivalent of a sulfonic acid to thereby produce a compound of formula 55a (See: FIG. 27B):

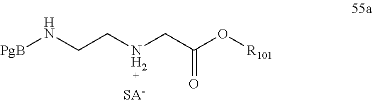

wherein, PgB and R₁₀₁ are defined above and SA⁻ is a sulfonate anion.

In some embodiments, the base-labile protecting group PgB is Fmoc. In some embodiments, the base-labile protecting group PgB is selected from the group consisting of: Nsc, Bsmoc, Nsmoc, ivDde, Fmoc*, Fmoc(2F), mio-Fmoc, dio-Fmoc, TCP, Pms, Esc, Sps and Cyoc.

In some embodiments, the sulfonate anion SA⁻ is produced from a sulfonic acid selected from the group consisting of: benzenesulfonic acid, naphthalenesulfonic acid, p-xylene-2-sulfonic acid, 2,4,5-trichlorobenzenesulfonic acid, 2,6-dimethylbenzenesulfonic acid, 2-mesitylenesulfonic acid (or dihydrate), 2-methylbenzene sulfonic acid, 2-ethylbenzenesulfonic acid, 2-isopropylbenzenesulfonic acid, 2,3-dimethylbenzenesulfonic acid, and 2,4,6-triisopropylbenzenesulfonic acid. In some embodiments, the sulfonate anion SA⁻ is produced from p-toluenesulfonic acid.

In some embodiments, SA⁻ is selected from benzenesulfonate, naphthalenesulfonate, p-toluenesulfonate, p-xylene-2-sulfonate, 2,4,5-trichlorobenzenesulfonate, 2,6-dimethylbenzenesulfonate, 2-mesitylenesulfonate, 2-mesitylenesulfonate dihydrate, 2-methylbenzene sulfonate, 2-ethylbenzenesulfonate, 2-isopropylbenzenesulfonate, 2,3-dimethylbenzenesulfonate, and 2,4,6-triisopropylbenzenesulfonate. In some embodiments, Sk is p-toluenesulfonate.

In some embodiments, in formula 53a, anion Y⁻ is selected from the group consisting of: I⁻, Br⁻, AcO⁻ (acetate), citrate or tosylate. In some embodiments, the anion Y⁻ is Cl⁻ or CF₃COO⁻ (trifluoroacetate).

In other embodiments, the present invention pertains to purified preparations of Backbone Esters and Backbone Ester Acid Salts and methods of providing the same. In some embodiments, a purified Backbone Ester preparation comprises at least 1 gram of a Backbone Ester (e.g., at least 2 grams, at least 3 grams, at least 4 grams, at least 5 grams, at least 10 grams, at least 15 grams, at least 20 grams, at least 30 grams, at least 40 grams, at least 50 grams, at least 75 grams, at least 100 grams or more Backbone Ester). In other embodiments, a purified Backbone Ester preparation comprises at least 1 gram of a Backbone Ester (e.g., at least 2 grams, at least 3 grams, at least 4 grams, at least 5 grams, at least 10 grams, at least 15 grams, at least 20 grams, at least 30 grams, at least 40 grams, at least 50 grams, at least 75 grams, at least 100 grams or more Backbone Ester).

In some embodiments, a purified Backbone Ester Acid Salt preparation comprises at least 1 gram of a Backbone Ester Acid Salt (e.g., at least 2 grams, at least 3 grams, at least 4 grams, at least 5 grams, at least 10 grams, at least 15 grams, at least 20 grams, at least 30 grams, at least 40 grams, at least 50 grams, at least 75 grams, at least 100 grams or more Backbone Ester Acid Salt). In other embodiments, a purified Backbone Ester Acid Salt preparation comprises at least 1 gram of a Backbone Ester Acid Salt (e.g., at least 2 grams, at least 3 grams, at least 4 grams, at least 5 grams, at least 10 grams, at least 15 grams, at least 20 grams, at least 30 grams, at least 40 grams, at least 50 grams, at least 75 grams, at least 100 grams or more Backbone Ester Acid Salt).

In some embodiments, the present invention comprises a method for providing a purified preparation of a Backbone Ester or a Backbone Ester Acid Salt. In some embodiments, the method comprises separating an impurity from the Backbone Ester. In some embodiments, the impurity comprises a reducing agent, an acid, or a solvent. In some embodiments, the purified preparation of the Backbone Ester comprises less than about 1 gram of an impurity (e.g., a reducing agent, an acid, or a solvent), for example, less than 0.5 grams, less than 0.1 grams, less than 0.05 grams, less than 0.01 grams, less than 0.005 grams, or less than 0.001 grams of an impurity (e.g., a reducing agent, an acid, or a solvent).

In another aspect, the present invention features a method of evaluating preparations of a Backbone Ester. Methods of evaluating said preparations may comprise acquiring, e.g., directly or indirectly, a value for the level of a particular component in the preparation. In some embodiment, the present invention features a method of evaluating a preparation of a a Backbone Ester comprising: a) acquiring, e.g., directly or indirectly, a value for the level of an impurity, e.g., by LCMS or GCMS; and b) evaluating the level of the impurity, e.g., by comparing the value of the level of the impurity with a reference value; thereby evaluating the preparation. In some embodiments, the impurity comprises a reducing agent, an acid, or a solvent. A reducing agent may be NaBH₃CN. An acid may be acetic acid. A solvent may be ethanol.

In another embodiment, the present invention features a method of evaluating a preparation of a Backbone Ester or a Backbone Ester Acid Salt comprising: a) acquiring, e.g., directly or indirectly, a value for the level of an impurity, e.g., by LCMS or GCMS; and b) evaluating the level of the impurity, e.g., by comparing the value of the level of the impurity with a reference value; thereby evaluating the preparation. In some embodiments, the impurity comprises an acid. In some embodiments, the acid is a sulfonic acid. In some embodiments, the sulfonic acid is selected from the group consisting of: p-toluenesulfonic acid, benzenesulfonic acid, naphthalenesulfonic acid, p-xylene-2-sulfonic acid, 2,4,5-trichlorobenzenesulfonic acid, 2,6-dimethylbenzenesulfonic acid, 2-mesitylenesulfonic acid, 2-mesitylenesulfonic acid dihydrate, 2-methylbenzene sulfonic acid, 2-ethylbenzenesulfonic acid, 2-isopropylbenzenesulfonic acid, 2,3-dimethylbenzenesulfonic acid, 2,4,6-trimethylbenzenesulfonic acid, and 2,4,6-triisopropylbenzenesulfonic acid. In some embodiments, the sulfonic acid is selected from the group consisting of: p-toluenesulfonic acid, benzenesulfonic acid, naphthalenesulfonic acid, p-xylene-2-sulfonic acid, 2,4,5-trichlorobenzenesulfonic acid, 2,6-dimethylbenzenesulfonic acid, 2-mesitylenesulfonic acid, 2-mesitylenesulfonic acid dihydrate, 2-methylbenzene sulfonic acid, 2-ethylbenzenesulfonic acid, 2-isopropylbenzenesulfonic acid, 2,3-dimethylbenzenesulfonic acid, and 2,4,6-triisopropylbenzenesulfonic acid.

In some embodiments, a reference value may be compared with the level of an impurity to determine the level of purity of a preparation, e.g., of a Backbone Ester or a Backbone Ester Acid Salt preparation. In some embodiments, a Backbone Ester preparation has a purity level of about 90%, about 95%, about 97.5%, about 99%, about 99.9%, or greater. In some embodiments, a Backbone Ester Acid Salt preparation has a purity level of about 90%, about 95%, about 97.5%, about 99%, about 99.9%, or greater.

III. Methods for Producing PNA Oligomers from PNA Monomers and PNA Monomer Esters

Described herein are methods of making PNA oligomers from PNA monomers and/or PNA Monomer Esters. In some embodiments, the present invention features a method of forming a PNA oligomer comprising a) providing a PNA Monomer Ester of formula (II) (e.g., formula II described herein); b) removing R₁ from the PNA Monomer Ester of formula (II) to form a PNA monomer and a liberated protecting group PgY; and c) contacting the PNA monomer with a PNA oligomer having a reactive N-terminus under conditions that allow for the formation of an amide bond between the PNA monomer and the PNA oligomer having the reactive N-terminus, thereby forming a (elongated) PNA oligomer.

The PNA oligomer may be prepared via solid phase synthesis or solution phase synthesis, e.g., using standard protocols. In some embodiments, the PNA oligomer is prepared using solid phase synthesis. In some embodiments, the method comprises linking multiple PNA monomers together on a solid support. In some embodiments, the PNA oligomer having a reactive N-terminus is linked by a linker to a solid support. In some embodiments, the linker comprises a covalent bond. Exemplary linkers may include an alkyl group, a polyethylene glycol group, an amine, or other functional group. In some embodiments, the linker comprises at least one PNA subunit.

In some embodiments, the method is carried out using an automated instrument. In some embodiments, the method is carried out in the solution phase.

In some embodiments, the liberated protecting group PgY comprises an alkenyl group. Without being bound by theory, the proposed deprotection of the PNA monomer entails unmasking the free carboxylic acid and formation of the corresponding liberated protecting group PgY, e.g., a haloethylene. Exemplary liberated protecting groups (PgY) include dibromoethylene, dichloroethylene, chloroethylene, bromoethylene, iodoethylene and ethylene.

A PNA oligomer may be prepared by iterative coupling of PNA monomers onto a solid support. In some embodiments, the method comprises d) providing a second PNA Monomer Ester of formula (II)) (e.g., formula II described herein); e) removing R₁ from the second PNA Monomer Ester of formula (II) to form a second PNA monomer; and f) contacting the second PNA monomer with a PNA oligomer comprising a reactive N-terminus under conditions that allow for the formation of an amide bond between the second PNA monomer and the PNA oligomer having the reactive N-terminus, thereby forming a (elongated) PNA oligomer. In some embodiments, the method comprises g) providing a third PNA Monomer Ester of formula (II)) (e.g., formula II described herein); h) removing R₁ from the third PNA monomer ester of formula (II) to form a third PNA monomer; and i) contacting the third PNA monomer with a PNA oligomer with a reactive N-terminus under conditions that allow for the formation of an amide bond between the third PNA monomer and the PNA oligomer having the reactive N-terminus, thereby forming a (elongated) PNA oligomer. In some embodiments, the conditions that allow for the formation of an amide bond comprise a coupling agent (e.g., DCC, EDC, HBTU or HATU). In some embodiments, the conditions that allow for the formation of an amide bond comprise at least a catalytic amount of DMAP.

In some embodiments, the PNA oligomer comprises at least 2, at least 3, at least 4, at least 5, at least 6, at least 7, at least 8, at least 9, or at least 10 PNA subunits. In some embodiments, the PNA oligomer comprises between 2 and 50 PNA subunits. In some embodiments, the PNA oligomer comprises between 10 and 50 PNA subunits. In some embodiments, the PNA oligomer comprises between 25 and 50 PNA subunits. In some embodiments, the PNA oligomer comprises between 30 and 45 PNA subunits. In some embodiments, the PNA oligomer comprises between 30 and 40 PNA subunits. In some embodiments, the PNA oligomer comprises between 35 and 40 PNA subunits.

In some embodiments, the PNA Monomer Ester of formula (II) (e.g., as described herein) for use in the method of forming a PNA oligomer comprises a nucleobase depicted in FIG. 2. FIG. 18a, or FIG. 18b. In some embodiments, the nucleobase is a naturally occurring nucleobase. In some embodiments, the nucleobase is a nonnaturally occurring nucleobase. In some embodiments, the nucleobase is selected from the group of adenine, guanine, thymine, cytosine, uracil, pseudoisocytosine, 2-thiopseudoisocytosine, 5-methylcytosine, 5-hydroxymethyl cytosine, xanthine, hypoxanthine, 2-aminoadenine (a.k.a. 2,6-diaminopurine), 2-thiouracil, 2-thiothymine, 2-thiocytosine, 5-chlorouracil, 5-bromouracil, 5-iodouracil, 5-chlorocytosine, 5-bromocytosine, 5-iodocytosine, 5-propynyl uracil, 5-propynyl cytosine, 6-azo uracil, 6-azo cytosine, 6-azo thymine, 7-methylguanine, 7-methyladenine, 8-azaguanine, 8-azaadenine, 7-deazaguanine, 7-deazaadenine, 3-deazaguanine, 3-deazaadenine, 7-deaza-8-aza guanine, 7-deaza-8-aza adenine, 5-propynyl uracil and 2-thio-5-propynyl uracil, including tautomeric forms of any of the foregoing

IV. Kits

In some embodiments, this invention pertains to kits. Kits are generally provided as a convenience wherein materials that naturally are used together are conveniently provided in amounts used for a particular application, often accompanied by instructions directed to performing that application. For example, the Backbone Esters or Backbone Ester Acid Salts compounds disclosed herein could be packaged with a nucleobase acetic acid and optionally a solvent useful for producing a PNA Monomer Ester. As another example, a kit could comprise a PNA Monomer Ester and a reducing agent (such as zinc or an organic phosphine) suitable to convert the PNA Monomer Ester to a PNA Monomer. This kit could optionally include a solvent suitable for performing said conversion.

In some embodiments, this invention pertains to a kit comprising a compound of formula VI, VI-T, VI-Ts, VI-Ts-A, VI-Ts-B, VI-Ts-C, VI-Ts-D, VI-Ts-E, VI-Ts-F, VI-Ts-G, VI-Ts-H, VI-Ts-I, VI-Ts-J, VI-Ts-K and/or VI-Ts-L, and (i) instructions; (ii) a base acetic acid; and/or (iii) a solvent.

### 6. Examples

Aspects of the present teachings can be further understood in light of the following examples, which should not be construed as limiting the scope of the present teachings in any way. Furthermore, it should be readily apparent to those of skill in the art that the following general procedures can be altered by variations on solvent, volumes and amounts of reagents in various steps to achieve optimal results for a particular compound without deviating from the scope and intent of the following guidance.

### Example 1: General Procedure for Making Esters of N-Protected Glycine (Compound 12—See: FIG. 19)

To N-protected glycine and the appropriate halogenated ethanol (e.g. 2,2,2-trichloroethanol, 2,2-dichloroethanol, 2-chloroethanol, 2,2,2-bromoethanol, 2,2-dibromoethanol, 2-bromoethanol or 2-iodoethanol; in a ratio of about 1 equivalent (eq.) of N-protected glycine (compound 10) per about 1-1.2 eq. of alcohol) was added DCM (generally in a ratio of about 2 to 3 mL DCM per mmol of N-protected glycine). This stirring solution was cooled in an ice bath for approximately 20 minutes and then a catalytic amount of DMAP (in a ratio of about 0.05 to 0.1 eq. per eq. of N-protected glycine) and carbodiimide (DCC or EDC in a ratio of 1.1-1.3 eq. per eq. of N-protected glycine) was added (order of addition of DMAP and DCC can be inverted). The reaction was allowed to proceed while stirring in an ice bath for about 2 hours, then allowed to warm to room temperature (RT). The reaction was often stirred overnight (or several days) but could be worked up after another 2-3 hours of stirring while warming to RT.

When EDC was used, the reaction was merely transferred to a separatory funnel, extracted; (i) twice with half-saturated KH₂PO₄; (ii) twice with 5% NaHCO₃; and one or more times with saturated NaCl (brine). The product was then dried over MgSO₄ (granular), filtered, and evaporated. This material was used in the next step without further purification or optionally could be purified by recrystallization before subsequent use.

When DCC was used (See: Ref C-19), the reaction was filtered to remove DCU and the filtrate was evaporated. The residue was redissolved in EtOAc in a ratio of about 2 to 4 mL per mmol of N-protected glycine (starting material). Enough EtOAc was added to ensure that the organic layer was the top layer and the layers would separate. This solution was generally extracted: (i) at least once with 5-10% aqueous citric acid; (ii) once or twice with saturated NaHCO₃ and/or 5% NaHCO₃; (iii) optionally with water; and (iv) at least once with brine. The product was then dried over MgSO₄ (granular), filtered, and evaporated. The solid product was generally crystallized from EtOAc/Hexanes (multiple crops collected) before being used in the next step.

### Example 2: General Procedure for Making Esters of N-Protected Chiral Amino Acids (Compound 13—See: FIG. 19)

Because activation of a carboxylic acid that is adjacent to a chiral center by use of DCC (or EDC) and DMAP can induce epimerization (loss of chiral purity), the condensation reaction between N-protected chiral amino acids (chiral AAs) and the halogenated alcohols is generally performed using a coupling agent (CA) known to minimize or eliminate epimerization (and thereby maintain chiral purity).

Generally, such esters were made by reacting the chiral N-protected amino acid (Compound 11) in a suitable solvent such as DCM or DMF by addition of an excess (e.g. 1.05-5 eq.) of a tertiary organic base such as TEA, NMM or DIPEA and a slight excess (e.g. 1.1-1.3 eq.) of the coupling agent (e.g. HATU or HBTU). A slight excess (e.g. 1.05-1.5 eq.) of the halogenated alcohol was then added and the reaction was monitored by thin layer chromatograph (TLC) until complete. The product was then worked up as discussed in Example 1, above. Several N-protected esters of chiral amino acids were prepared using this general procedure as summarized in Table 1B, below, where yield data is also provided.

General Structure of Products Generated (See: FIG. 19):

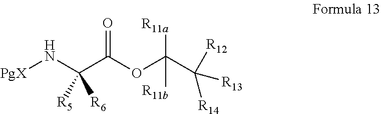

wherein PgX, R₅, R₆, R₁₁ₐ, R_{11b}, R₁₂, R₁₃ and R₁₄ are as previously defined (and as used in Table 1A, below, except that for clarity, R₁₁ₐ and R_{11b} are each defined as being independently H, D, F, C₁-C₆ alkyl, C₃-C₆ cycloalkyl or aryl).

TABLE 1A
Table of Some Exemplary (non-limiting) Compounds

| Cpd. # | PgX | R₅ | R₆ | R₁₁ₐ | R_{11b} | R₁₂ | R₁₃ | R₁₄ | CA^{‡} |
|---|---|---|---|---|---|---|---|---|---|
| 13a | Boc | H | H | H | H | Cl | Cl | Cl | EDC |
| 13a | Boc | H | H | H | H | Cl | Cl | Cl | DCC |
| 13b | Boc | H | H | H | H | Br | Br | Br | DCC |
| 13c | Boc | H | H | H | H | H | I | H | EDC |
| 13d | Boc | CH₃ | H | H | H | Br | Br | Br | HBTU |
| 13e | Boc | H | CH₃ | H | H | Br | Br | Br | HBTU |
| 13f | Boc | Met | H | H | H | Br | Br | Br | HBTU |
| 13g | Boc | H | Met | H | H | Br | Br | Br | HBTU |
| 13h | Fmoc | Lys^{(Boc)} | H | H | H | Br | Br | Br | HBTU |
| 13i | Fmoc | H | Lys^{(Boc)} | H | H | Br | Br | Br | HBTU |
| 13j | Fmoc | Ser^{(tBu)} | H | H | H | Br | Br | Br | HBTU |
| 13k | Fmoc | H | Ser^{(tBu)} | H | H | Br | Br | Br | HBTU |
| 13l | Fmoc | Glu^{(tBu)} | H | H | H | Br | Br | Br | HBTU |
| 13m | Fmoc | H | Glu^{(tBu)} | H | H | Br | Br | Br | HBTU |
| 13n | Fmoc | Arg^{(Pbf}) | H | H | H | Br | Br | Br | HBTU |
| 13o | Fmoc | H | Arg^{(Pbf)} | H | H | Cl | Cl | Cl | HBTU |
| 13p | Fmoc | H | Arg^{(Pbf)} | H | H | Br | Br | Br | HBTU |
| 13q | Fmoc | Cys^{(Trt)} | H | H | H | Br | Br | Br | HBTU |
| 13r | Fmoc | H | Cys^{(Trt)} | H | H | Br | Br | Br | HBTU |
| 13s | Fmoc | His^{(Trt)} | H | H | H | Br | Br | Br | HBTU |
| 13t | Fmoc | H | His^{(Trt)} | H | H | Br | Br | Br | HBTU |
| 13u | Fmoc | Try^{(tBu)} | H | H | H | Br | Br | Br | HBTU |
| 13v | Fmoc | H | Tyr^{(tBu)} | H | H | Br | Br | Br | HBTU |
| 13w | tfa | H | H | H | H | Br | Br | Br | EDC |
| 13x | Boc | H | H | CH₃ | H | H | Br | H | EDC |

CA^{‡} = Coupling Agent

TABLE 1B
Table of Products Generated

| Compound | Starting Protected Glycine | | | mM of | mM of | |
|---|---|---|---|---|---|---|
| No. | or Chiral AA (SM) | Alcohol | EDC/ DCC | SM | Product | Yield |
| 13a | N-(Boc)glycine | 2,2,2-trichloroethanol | EDC | 40 | 37.35 | 93.4% |
| 13a | N-(Boc)glycine | 2,2,2-trichloroethanol | DCC | 200 | 175 | 87.9% |
| 13b | N-(Boc)glycine | 2,2,2-tribromoethanol | DCC | 500 | 410.4 | 82.1% |
| 13c | N-(Boc)glycine | 2-iodoethanol | DCC | 50 | 47.2 | 94% |
| 13d | N-(Boc)-L-alanine | 2,2,2-tribromoethanol | HBTU | 100 | 78 | 78% |
| 13e | N-(Boc)-D-alanine | 2,2,2-tribromoethanol | HBTU | 60 | 41 | 68.3% |
| 13f | N-(Boc)-L-methionine | 2,2,2-tribromoethanol | HBTU | 35 | 31.3 | 89% |
| 13g | N-(Boc)-D-methionine | 2,2,2-tribromoethanol | HBTU | 100 | 95.6 | 95.6% |
| 13n | N-(Fmoc)-L-Arg^{(Pbf)} | 2,2,2-tribromoethanol | HBTU | 30 | 13.8 | 46% |
| 13o | N-(Fmoc)-D-Arg^{(Pbf)} | 2,2,2-trichloroethanol | HBTU | 2.5 | 0.84 | 33.6% |
| 13p | N-(Fmoc)-D-Arg^{(Pbf)} | 2,2,2-tribromoethanol | HBTU | 30 | 3.2 | 50%* |
| 13w | N-(tfa)-glycine | 2,2,2-tribromoethanol | EDC | 125 | 12.6 | 10% |

*Obtained from column chromatography of a 6.0 g fraction of the crude product.

### Example 3: General Procedure for Producing TFA Salts of Amino Acid Esters from N-(Boc)-Protected Amino Acids (See: FIG. 19)

N-(Boc) protected amino acids are generally selected as the starting material for glycine and other amino acids comprising alkyl side chains (e.g. methyl) or if one intends to produce an amino acid ester of an amino acid that contains a base-labile side chain protecting group. To the N-(Boc) protected amino acid was added DCM (in a ratio of about 1 to 1.5 mL per mmol of N-(Boc) protected amino acid). Other solvents compatible with TFA can also be used if so desired. This solution was allowed to cool in an ice bath for 10-30 minutes and then to the stirring solution was added TFA in a volume equal to the volume of added DCM. The ice bath was removed and the reaction was allowed to stir while warming to room temperature (RT) over 30 minutes. Solvent was then removed under reduced pressure. If desirable to remove residual TFA, the residue could be co-evaporated one or more times from toluene. However, in many cases this step was eliminated and the residue was triturated by addition to (or addition of) diethyl ether and/or hexanes.

For example, the TFA salt of the 2,2,2-tribromoethyl ester of glycine was triturated by the addition of diethyl ether (and stirring) and the salt was allowed to stir in the ether for 1-2 hours before being collected by vacuum filtration. Conversely, the TFA salt of the 2,2,2-trichloroethyl ester of glycine was co-evaporated twice from toluene (about 2.5-3.0 mL of toluene per mmol of N-(Boc) protected amino acid starting material) and then dissolved in diethyl ether (about 1.2-1.4 mL per mmol of N-(Boc) protected amino acid starting material). The TFA salt then crashed out of solution upon addition of hexanes (about 1.5-1.7 mL per mmol of N-(Boc) protected amino acid starting material) to the briskly stirring solution. The TFA salt was then collected by vacuum filtration.

General Structure of Products Generated (See: FIG. 19):

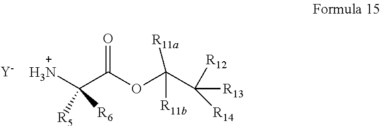

wherein Y⁻, R₅, R₆, R₁₁ₐ, R_{11b}, R₁₂, R₁₃ and R₁₄ are previously defined and as used in Table 2A below.

TABLE 2A
Table of Some Exemplary (non-limiting) Compounds

| Cpd. # | Y⁻ | R₅ | R₆ | R₁₁ₐ | R_{11b} | R₁₂ | R₁₃ | R₁₄ |
|---|---|---|---|---|---|---|---|---|
| 15a | TFA⁻ | H | H | H | H | Cl | Cl | Cl |
| 15b | TFA⁻ | H | H | H | H | Br | Br | Br |
| 15c | TFA⁻ | H | H | H | H | H | I | H |
| 15d | TFA⁻ | CH₃ | H | H | H | Br | Br | Br |
| 15e | TFA⁻ | H | CH₃ | H | H | Br | Br | Br |
| 15f | TFA⁻ | H | H | H | H | Br | Br | Br |
| 15g | TFA⁻ | H | Met | H | H | Br | Br | Br |
| 15ba | TFA⁻ | Val | H | H | H | Br | Br | Br |
| 15bb | TFA⁻ | H | Val | H | H | Br | Br | Br |
| 15bc | TFA⁻ | Phe | H | H | H | Br | Br | Br |
| 15bd | TFA⁻ | H | Phe | H | H | Br | Br | Br |
| 15be | TFA⁻ | Ile | H | H | H | Br | Br | Br |
| 15bf | TFA⁻ | H | Ile | H | H | Br | Br | Br |
| 15bg | TFA⁻ | Leu | H | H | H | Br | Br | Br |
| 15bh | TFA⁻ | H | Leu | H | H | Br | Br | Br |
| 15n | TFA⁻ | Arg | H | H | H | Br | Br | Br |
| 15p | TFA⁻ | H | Arg | H | H | Br | Br | Br |

The abbreviations Met, Val, Phe, Ile, Leu and Arg as used in Table 2A refer to the side chain of the amino acid indicated by use of the three letter code abbreviation.

TABLE 2B
Table of Products Generated

| Compound | | | Acid | mM of | mM of | |
|---|---|---|---|---|---|---|
| No. | Amino acid | Ester | Salt | SM | Product | Yield |
| 15a | glycine | 2,2,2-trichloroethanol | TFA | 37 | 36 | 98% |
| 15b | glycine | 2,2,2-tribromoethanol | TFA | 350 | 334 | 95.5% |
| 15c | glycine | 2-iodoethanol | TFA | 40 | 37.6 | 94% |
| 15d | L-alanine | 2,2,2-tribromoethanol | TFA | 70 | 68 | 97% |
| 15e | D-alanine | 2,2,2-tribromoethanol | TFA | 35 | 34 | 97% |
| 15f | L-methionine | 2,2,2-tribromoethanol | TFA | 31 | 28.3 | 91.4% |
| 15g | D-methionine | 2,2,2-tribromoethanol | TFA | 95.6 | 76.4 | 80% |

### Example 4: General Procedure for Producing HOAc, TFA or HCl Salts of Amino Acid Esters from N-(Fmoc)-Protected Amino Acids (See: FIG. 19)

N-(Fmoc) protected amino acids are generally selected as the starting material if one intends to produce an amino acid ester of an amino acid that contains an acid-labile side chain protecting group. To the N-(Fmoc) protected amino acid is added at least enough of a solution of 20% (v/v) piperidine in DMF to completely dissolve the N-(Fmoc) protected amino acid (For example, use 100 ml of 20% (v/v) piperidine (or 1% (v/v) of 1,8-Diazabicyclo[5.4.0]undec-7-ene “DBU”) in DMF for 20 mmol of N-(Fmoc) protected amino acid). This solution is allowed to stir at room temperature until TLC analysis indicates complete removal of the Fmoc group. Solvent is then removed under reduced pressure using a rotoevaporator. Excess piperidine can be removed by co-evaporation several times with water followed by co-evaporation from cyclohexane to remove residual water (these are compounds of formula 14 (See: FIG. 19)).

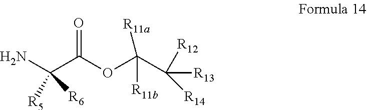

wherein R₅, R₆, R₁₁ₐ, R_{11b}, R₁₂, R₁₃ and R₁₄ are previously defined and as used in Table 3A below.

The residue can be dissolved in diethyl ether or other ether-based solvent (e.g. THF or 1,4-dioxane) and then at least one equivalent of acid (e.g. acetic acid (HOAc), TFA or HCl (e.g. from a solution of HCl dissolved in ether)) can be added to produce the acid salt (e.g. HOAc, TFA or HCl salt, respectively) of the amino acid ester (these have the formula 15, above). In general, a large excess of added acid is avoided to thereby reduce the likelihood of deprotection of the acid labile side chain protecting group. This process is expected to provide a compound of formula 15.

TABLE 3A
Table of Products Generated

| Cpd. # | Y⁻ | R₅ | R₆ | R₁₁ₐ | R_{11b} | R₁₂ | R₁₃ | R₁₄ |
|---|---|---|---|---|---|---|---|---|
| 15h | AcO⁻ | Lys^{(Boc)} | H | H | H | Br | Br | Br |
| 15i | AcO⁻ | H | Lys^{(Boc)} | H | H | Br | Br | Br |
| 15j | AcO⁻ | Ser^{(tBu)} | H | H | H | Br | Br | Br |
| 15k | AcO⁻ | H | Ser^{(tBu)} | H | H | Br | Br | Br |
| 15l | AcO⁻ | Glu^{(tBu)} | H | H | H | Br | Br | Br |
| 15m | AcO⁻ | H | Glu^{(tBu)} | H | H | Br | Br | Br |
| 15n | AcO⁻ | Arg^{(Pbf)} | H | H | H | Br | Br | Br |
| 15o | AcO⁻ | H | Arg^{(Pbf)} | H | H | Cl | Cl | Cl |
| 15p | AcO⁻ | H | Arg^{(Pbf)} | H | H | Br | Br | Br |
| 15q | AcO⁻ | Cys^{(Trt)} | H | H | H | Br | Br | Br |
| 15r | AcO⁻ | H | Cys^{(Trt)} | H | H | Br | Br | Br |
| 15s | AcO⁻ | His^{(Trt)} | H | H | H | Br | Br | Br |
| 15t | AcO⁻ | H | His^{(Trt)} | H | H | Br | Br | Br |
| 15u | AcO⁻ | Try^{(tBu)} | H | H | H | Br | Br | Br |
| 15v | AcO⁻ | H | Tyr^{(tBu)} | H | H | Br | Br | Br |

### Example 5: Synthesis of N-Protected aminoacetaldehyde—Formula 3-1

Part 1: Synthesis of N-protected 3-amino-1,2-propanediol—Formula 2 (See: FIG. 20)

For Fmoc protected 3-amino-1,2-propanediol, 9-fluorenylmethoxysuccinimidyl carbonate (Fmoc-O-Su) was suspended in acetone (about 1.2 mL acetone per mmol Fmoc-O-Su) with stirring. To the stirring solution at RT was added dropwise a solution containing 3-amino-1,2-propanediol (about 1.1 mmol per mmol of Fmoc-O-Su) dissolved in a mixture of acetone and water (about 4 to 1 acetone to water; and in a ratio of about 0.8-1.0 mL per mmol of 3-amino-1,2-propanediol—but other ratios will work as well). When complete, a solution containing NaHCO₃ and Na₂CO₃ (in a ratio of about 1 mmol NaHCO₃ and 0.5 mmol Na₂CO₃ per mmol of Fmoc-O-Su) dissolved in deionized water (in a ratio of about 1 mL deionized water per 1 mL of acetone originally added to the Fmoc-O-Su) was added dropwise to the stirring mixture. After stirring and analysis by TLC (indicating the reaction was complete), a solution containing enough HCl (dissolved in about 0.3 mL water per 1 mL of acetone originally added to the Fmoc-O-Su) to completely neutralize the NaHCO₃ and Na₂CO₃ was added dropwise over 30 minutes to one hour. The reaction was then concentrated on a rotoevaporator to remove acetone and the residue partitioned with EtOAc/deionized water/acetone (4/2/0.5) in a ratio of about 2.2 mL of this mixture per 1 mL of acetone originally added to the Fmoc-O-Su). The layers were separated and the aqueous layer extracted 3 times with more EtOAc. The combined organic layers were then extracted with a solution containing 3 parts brine and one part water. The organic layer was then dried over MgSO₄ (granular), filtered and evaporated to a solid. The product was recrystallized from 9/1 acetonitrile/water.

For Boc protected 3-amino-1,2-propanediol, the 3-amino-1,2-propanediol can be reacted at RT with a small excess (e.g. 1.02-1.1 eq.) of di-t-butyl dicarbonate (a.k.a. Boc anhydride) in an aprotic solvent such as DCM or THF. No base is needed and in some cases the reaction can be driven to completion by heating overnight. The product of the reaction can then be evaporated and used without further purification.

General Structure of Products Generated (See: FIG. 20):

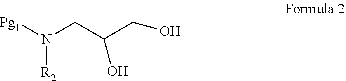

TABLE 4B
Table of Products Generated (including examples to be produced)

| Compound | Starting | | mM of | mM of | Yield of |
|---|---|---|---|---|---|
| No. | Material (SM) | Pg₁ | SM | Product | Product |
| 2a | 3-Amino-1,2- | Fmoc | 250 | 180 | 72% |
| | propanediol | | | | |

Part 2: Oxidation of N-Protected aminopropanediol to N-Protected aminoacetaldehyde (Formula 3-1; See: FIGS. 20)

To N-[Fmoc-(3-Amino)]-1,2-propanediol was added ethyl acetate (in a ratio of about 5-8 mL per mmol of N-[Fmoc-(3-Amino)]-1,2-propanediol) and ice (measured using a beaker) in a ratio of about 8-12 mL ice per equivalent of N-Fmoc-(3-Amino)-1,2-propanediol). The mixture was stirred using a mechanical stirrer. To the stirring mixture was added NaIO₄ (in a ratio of about 1.5-2 equivalents per equivalent of N-Fmoc-(3-Amino)-1,2-propanediol). After stirring for about 5 minutes, DCM (in a ratio of about 2 mL per mmol of N-Fmoc-(3-Amino)-1,2-propanediol) was added and the reaction was allowed to stir for about 1 hour in the ice bath and then the ice bath was removed. The reaction was then allowed to stir while warming to RT until TLC indicated essentially complete consumption of the starting material (about 2.5-3.5 hours). Additional NaIO₄ was added as needed until the N-Fmoc-(3-Amino)-1,2-propanediol was essentially consumed. When complete, sodium chloride was added to the stirring mixture (in a ratio of about 6-7 mmol NaCl per mmol of N-[Fmoc-(3-Amino)]-1,2-propanediol). After stirring for about 5 minutes to dissolve the NaCl, the entire contents of the flask was transferred to an appropriately sized separatory funnel and the layers were separated. The organic layer was then and washed: (i) at least once with of 5% NaHCO₃; and (ii) then at least once with brine. The organic layer was dried over MgSO₄ (granular), filtered, and evaporated. The N-(Fmoc)-aminoacetaldehyde was a solid and was be used in the reductive amination without further purification. This material could be stored at −20° C.

This general procedure can also be used to prepare the N-(Boc)-aminoacetaldehyde suitable for use without further purification. Generally, however, for the N-[Boc-(3-Amino)]-1,2-propanediol, only DCM is used in the reaction (not a mix of ethyl acetate and DCM) in roughly the same total concentration of organic to aqueous (ice) except that the reaction is not allowed to warm to RT and is always kept cold by precooling the extraction mixtures. The N-(Boc)-aminoacetaldehyde can be used in a reductive amination to make the N-Boc protected backbone ester, whereas the N-(Fmoc)-aminoacetaldehyde can be used in the reductive amination to prepare the N-Fmoc protected backbone ester.

General Structure of Products Generated (See: FIG. 20):

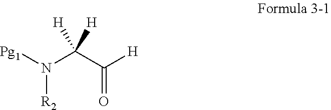

wherein, Pg₁ and R₂ are previously defined.

TABLE 5B
Table of Products Generated (including examples to be produced)

| Compound | Starting | mM of | mM of | Yield of |
|---|---|---|---|---|
| No. | Material (SM) | SM | Product | Product |
| 3-1a | N—Fmoc-(3-Amino)- | 30 | 30.1 | 100.3% |
| | 1,2-propanediol | | | |
| 3-1a | N—Fmoc-(3-Amino)- | 100 | 99 | 99% |
| | 1,2-propanediol | | | |

### Example 6: Preparation of Chiral N-Protected Amino Alcohols from Amino Alcohols—Formula 6 (See: FIG. 20)

Amino alcohol derivatives (both unprotected, N-protected and/or side chain protected) of common amino acids are available from commercial sources such as Chem Impex and Bachem. For example: L-alaninol (P/N 03169), D-alaninol (P/N 03170); L-methioninol (P/N 03204); D-methioninol; (P/N 03205); Boc-L-methioninol (P/N 03206); Fmoc-γ-tert-butyl ester-L-glutamol (P/N 03186); Boc-O-benzyl-L-serinol (P/N 03220) and Fmoc-O-tert-butyl-L-serinol (P/N 03222) are all commercially available from Chem Impex International, Inc. and other vendors of amino acid reagents.

Suitable N-protected amino alcohols (e.g. Fmoc and Boc) can be obtained by reacting an amino alcohol with a desired protecting group precursor that protects the amine group with the desired protecting group Pg₁. For example, N-Fmoc protected amino alcohols were prepared (in an Erlenmeyer flask) by suspending/dissolving Fmoc-O-Su in acetone (in a ratio of about 2.5-6 mL acetone per mmol of Fmoc-O-Su) with stirring. To this briskly stirring solution was added dropwise a solution of the amino alcohol (in a ratio of about 1 to 1.2 eq. per mmol of Fmoc-O-Su) dissolved in acetone (in a ratio of about 0.4-1.2 mL acetone per mmol of the amino alcohol) and occasionally some water if the amino alcohol is not completely soluble in the acetone alone. When addition was complete, a solution containing NaHCO₃ and Na₂CO₃ (in a ratio of about 1 to 1.1 mmol NaHCO₃ and 0.5 to 0.55 mmol Na₂CO₃ per mmol of Fmoc-O-Su) dissolved in deionized water (in a ratio of about 1 mL deionized water per 1 mL of acetone originally added to the Fmoc-O-Su) was added dropwise to the stirring reaction. After stirring and analysis by TLC (indicating complete reaction), a solution containing enough HCl (dissolved in about 0.3 mL water per 1 mL of acetone originally added to the Fmoc-O-Su) to completely neutralize the NaHCO₃ and Na₂CO₃ was added dropwise over 30 minutes to one hour. The pH of the solution was then adjusted to approximately 4-5 (pH paper) by addition of 1N HCl. The flask was then heated on a hot plate stirrer until the solid dissolved. The solution was then allowed to cool overnight and the product crystallized. The crystalline product was then collected by vacuum filtration. The product was then optionally recrystallized (usually by a mixture of acetonitrile and water) to the desired level of purity.

General Structure of Products Generated:

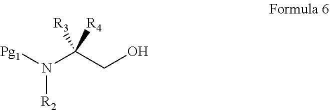

wherein, Pg₁, R₂, R₃ and R₄ are previously defined.

TABLE 6A
Table of Some Exemplary (non-limiting) Compounds

| Cpd. # | Pg₁ | R₂ | R₃ | R₄ | L or D | Amino Acid |
|---|---|---|---|---|---|---|
| 6a-1 | Fmoc | H | CH₃ | H | L | Ala |
| 6a-2 | Boc | H | CH₃ | H | L | Ala |
| 6b-1 | Fmoc | H | H | CH₃ | D | Ala |
| 6b-2 | Boc | H | H | CH₃ | D | Ala |
| 6c-1 | Fmoc | H | CH₂CH₂SCH₃ | H | L | Met |
| 6c-2 | Boc | H | CH₂CH₂SCH₃ | H | L | Met |
| 6d-1 | Fmoc | H | H | CH₂CH₂SCH₃ | D | Met |
| 6d-2 | Boc | H | H | CH₂CH₂SCH₃ | D | Met |
| 6e-1 | Fmoc | H | CH(CH₃)₂ | H | L | Val |
| 6e-2 | Boc | H | CH(CH₃)₂ | H | L | Val |
| 6f-1 | Fmoc | H | H | CH(CH₃)₂ | D | Val |
| 6f-2 | Boc | H | H | CH(CH₃)₂ | D | Val |
| 6g-1 | Fmoc | H | CH₂CH(CH₃)₂ | H | L | Leu |
| 6g-2 | Boc | H | CH₂CH(CH₃)₂ | H | L | Leu |
| 6h-1 | Fmoc | H | H | CH₂CH(CH₃)₂ | D | Leu |
| 6h-2 | Boc | H | H | CH₂CH(CH₃)₂ | D | Leu |
| 6i-1 | Fmoc | H | CH(CH₃)(O-Bn) | H | L | Thr(Bn) |
| 6i-2 | Fmoc | H | CH₂(S-mBn) | H | L | Cys(mBn) |

TABLE 6B
Table of Products Generated (including examples to be produced)

| Compound | Starting | | mM of | mM of | Yield of |
|---|---|---|---|---|---|
| No. | Material (SM) | Pg₁ | Fmoc—O-Su | Product | Product |
| 6a-1 | L-alaninol | Fmoc | 400 | 356 | 89% |
| 6b-1 | D-alaninol | Fmoc | 150 | 129 | 86% |
| 6c-1 | L-methioninol | Fmoc | 95 | 65.1 | 69% |
| 6d-1 | D-methioninol | Fmoc | 95 | 68.9 | 72% |
| 6e-1 | L-valinol | Fmoc | 100 | 70 | 70% |
| 6h-1 | L-leucinol | Fmoc | 100 | 78 | 78% |

### Example 7: Reduction of Chiral N-Protected Amino Acids to N-Protected Amino Alcohols—Formula 6 (See: FIG. 20)

Several literature methods have been shown to produce N-protected chiral amino alcohols from N-protected chiral amino acids (See for example: Refs. C-1, C-3, C-5, C-15 and C-24). These procedures can be selected to produce N-base-labile protected (e.g. Fmoc protected) chiral amino alcohols or N-acid-labile protected (e.g. Boc protected) chiral amino alcohols. These chiral amino alcohols can (depending on the methodology selected) also produce N-protected chiral amino alcohols bearing side chain protecting groups. As noted above, many of these compounds are commercially available and therefore need not be produced (See Table 7A).

By way of an example, the procedure of Rodriquez et al. (Ref. C-21) was followed to produce both the D- and L-enantiomers of Fmoc methionine. In each case, 25 mmol of N-Fmoc methionine was dissolved/suspended in 25 mL of 1,2-dimethoxyethane (“DME”) and this solution was cooled in an ice/salt bath to about −5-10° C. (See: Table 7B). Then, a slight excess (25.5-26 mmol) of NMM was added and allowed to stir for about 1-3 minutes before isobutyl chloroformate (25.5-26 mmol) was added. After a few minutes of reacting, the reaction was filtered to remove the N-methylmorpholine hydrochloride. The filter cake was then washed several times with 5 mL portions of DME. To the filtrate was added a solution of 39-40 mmol of sodium borohydride dissolved in 13 mL deionized water with mixing and then immediately thereafter (400-650 mL) of deionized water was added to produce a white solid. This white solid was collected by vacuum filtration and the cake washed with water and then hexanes. The product was dried under high vacuum. According to Rodriquez, this procedure is generally applicable to the other amino acids. Indeed, this general procedure was also shown to be effective to produce both L- and D-enantiomers of suitably protected serine (See: Table 7B).

General Structure of Products Generated:

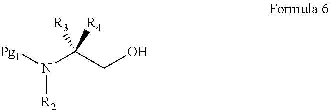

wherein, Pg₁, R₂, R₃ and R₄ are previously defined.

TABLE 7A
Table of Some Commercially Available Compounds

| Cpd. # | Pg¹ | R₂ | R₃ | R₄ | L or D | Amino Acid |
|---|---|---|---|---|---|---|
| 6a-1 | Fmoc | H | H | CH₃ | L | Ala |
| 6a-2 | Boc | H | H | CH₃ | L | Ala |
| 6b-1 | Fmoc | H | CH₃ | H | D | Ala |
| 6b-2 | Boc | H | CH₃ | H | D | Ala |
| 6c-1 | Fmoc | H | H | CH₂CH₂SCH₃ | L | Met |
| 6c-2 | Boc | H | H | CH₂CH₂SCH₃ | L | Met |
| 6d-1 | Fmoc | H | CH₂CH₂SCH₃ | H | D | Met |
| 6d-2 | Boc | H | CH₂CH₂SCH₃ | H | D | Met |
| 6e-1 | Fmoc | H | H | CH(CH₃)₂ | L | Val |
| 6e-2 | Boc | H | H | CH(CH₃)₂ | L | Val |
| 6f-1 | Fmoc | H | CH(CH₃)₂ | H | D | Val |
| 6f-2 | Boc | H | CH(CH₃)₂ | H | D | Val |
| 6g-1 | Fmoc | H | H | CH₂CH(CH₃)₂ | L | Leu |
| 6g-2 | Boc | H | H | CH₂CH(CH₃)₂ | L | Leu |
| 6h-1 | Fmoc | H | CH₂CH(CH₃)₂ | H | D | Leu |
| 6h-2 | Boc | H | CH₂CH(CH₃)₂ | H | D | Leu |
| 6i-1 | Fmoc | H | H | CH(CH₃)(O-Bn) | L | Thr(Bn) |
| 6i-2 | Fmoc | H | H | CH₂(S-mBn) | L | Cys(mBn) |
| 6j | Fmoc | H | H | CH₂O-tBu | L | Ser(OtBu) |
| 6k | Fmoc | H | CH₂O-tBu | H | D | Ser(OtBu) |

TABLE 7B
Table of Products Generated (including examples to be produced)

| Compound | Starting | | mM of | mM of | Yield of |
|---|---|---|---|---|---|
| No. | Material (SM) | Pg₁ | SM | Product | Product |
| 6c-1 | Fmoc-L-methionine | Fmoc | 25 | 22.2 | 89% |
| 6d-1 | Fmoc-D-methionine | Fmoc | 25 | 19.3 | 77% |
| 6j | Fmoc-L-(O-tBu)- | Fmoc | 50 | 30.4 | 61% |
| | serine | | | | |
| 6k | Fmoc-D-(O-tBu)- | Fmoc | 125 | 63.4 | 51% |
| | serine | | | | |

### Example 8: Preparation of N-Protected Chiral Aldehydes of Amino Acids—Formula 3 (See: FIG. 20)

Compounds of Formula 3-1 (N-protected aminoacetaldehyde) are achiral and are essentially the product of this procedure when glycine is used as the starting amino acid according to Example 7. Because of its ease, N-protected aminoacetaldehyde is preferably prepared according to the procedure in Example 5. For all aldehydes with a chiral center (e.g. aldehydes of N-protected D or L amino acids), this Example 8 is preferred.

There are reports of using Dess-Martin Periodinane to produce N-protected-aminoaldehydes of high enantiomeric excess (ee) from the corresponding N-protected amino alcohols (which as shown above are readily available from commercial sources or easily produced directly from available starting materials, including naturally occurring chiral amino acids, and chiral amino alcohols (Also ee: Section 4(IX)(b), above). This process can be carried out on amino acids comprising both acid-labile and base-labile N-protecting groups (as Pg₁). The following procedure is adapted from (but follows closely) the procedure of Myers et al., Ref. C-18.

To the N-protected amino alcohol was added wet (Ref. C-17) DCM (in a ratio of from about 3.3 to 5.7 mL per mmol of N-protected amino alcohol (more wet DCM was needed to solubilize the N-protected methioninol derivatives). This solution was cooled in an ice bath for about 10-30 minutes before proceeding. To the stirring solution was then added about 1.5 to 2.1 equivalents of Dess-Martin Periodinane (DMP—divided into 2-5 portions and added portionwise over 10-20 minutes). The reaction was monitored by TLC and additional DMP was added until essentially all the starting N-protected amino alcohol was consumed. Additional wet DCM was also added several times during the reaction (See: Ref. C-18). Generally, the reaction was done in 1-2 hours.

When deemed complete, the reaction mixture was poured into a briskly stirring (preferably cooled in an ice bath) mixture of diethyl ether and an aqueous solution of sodium thiosulfate and NaHCO₃ as described by Myers et al (Ref. C-18). The remainder of the workup was also carried out essentially as described by Myers et al (Ref. C-18). The product N-protected aldehyde was generally used the same day in the reductive amination (discussed below in Example 9) as isolated from the extraction, without any further purification.

General Structure of Products Generated:

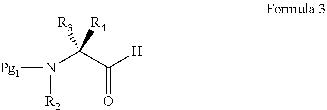

wherein, Pg₁, R₂, R₃ and R₄ are previously defined.

TABLE 8B
Table of Products Generated (including examples to be produced)

| Cpd. # | From Amino Acid | Pg¹ | R₂ | R₃ | R₄ | % Yield |
|---|---|---|---|---|---|---|
| 3-1 | L-alanine | Fmoc | H | H | CH₃ | 103 |
| 3-3 | L-methionine | Fmoc | H | H | CH₃ | 95 |
| 3-4 | D-methionine | Fmoc | H | H | CH₃ | 130 |
| 3-7 | L-serine | Fmoc | H | H | CH₂O-tBu | 102 |
| 3-8 | D-serine | Fmoc | H | CH₂O-tBu | H | 104 |

### Example 9A: Reductive Aminations to Produce Backbones—Formulas V, Vb & VI and VIb—See: FIG. 21

The general procedure used for producing Backbone Esters and Backbone Ester Acid Salts is illustrated in FIG. 21. Generally, the reaction involves reacting an aldehyde according to formula 3 with an amino acid ester salt (salt of the amine) according to formula 15 in the presence of a reducing agent such as sodium cyanoborohydride (NaBH₃CN) in ethanol at low temperature (−10 to 0° C.). This procedure is adapted from the procedures described in References C-8, C-9 and C-22 (Huang, Huang and Salvi).

The amino acid ester salt (in a ratio of about 1.05 to 2 equivalents per mmol of aldehyde) was dissolved/suspended in ethanol (EtOH—about 3-7 mL per mole of aldehyde—see below) and this solution was cooled in an ice/salt bath to −15 to 0° C. Glacial acetic acid and optionally an organic base like NMM or DIPEA was added while the solution cooled to −10 to 0° C. (the glacial acetic acid was added in a ratio of about 1.4 to 4 equivalents per mmol of aldehyde and the organic base was generally added in about 0.9-1.0 equivalent per mmol of amino acid ester salt). When sufficiently cool, the aldehyde (prepared as described in Examples 5 or 8) was added to the stirring solution (generally slow to dissolve) and the reaction was maintained at −10 to 0° C. while the aldehyde slowly dissolved and the reaction was monitored by TLC. The sodium cyanoborohydride (NaBH₃CN) was, in some cases, added immediately before the aldehyde was added and in some cases immediately after. Ethanol was selected as the solvent because the NaBH₃CN was sufficiently soluble in EtOH but this solvent avoided the problems with transesterification observed with methanol. Lowering the reaction temperature to −10 to 0° C. helped to avoid the bis-addition of aldehyde as reported by Salvi.

When the reaction was deemed complete by TLC, the ethanol was removed under reduced pressure and the residue was partitioned in EtOAc and deionized water or one-half saturated KH₂PO₄. The EtOAc layer was then washed: (i) at least once with one-half saturated KH₂PO₄, (ii) one or more times with 5% NaHCO₃ and/or saturated NaHCO₃, and (iii) at least once with brine (CAUTION: Always discard cyanide containing waste to a special cyanide containing waste stream and do not combine with strong acids so as to avoid forming toxic HCN gas that is lethal). The EtOAc layer was then dried over MgSO₄ (granular), filtered and evaporated. This residue was immediately loaded onto a silica gel column and purified by chromatography using EtOAc/hexanes running an EtOAc gradient (or DCM/MeOH running a MeOH gradient). Fractions were collected and pooled based on TLC analysis. This process produced compounds of general formula V (and Vb).

In Applicants experience, when Pg₁ is Fmoc, compounds of general formula V (and Vb) are unstable for even short periods of time (as determined by TLC). This instability is likely attributable to the basicity of the secondary amine, which appears to promote both: 1) removal of the Fmoc protecting group; and 2) migration of the Fmoc group from the primary amine to the secondary amine. Accordingly, Applicants found it judicious to immediately stabilize the Backbone Ester by producing the acid salt of the secondary amine, thereby rendering it temporarily unreactive.

Generally, the acid salt of the Backbone Ester was generated by dissolving it in a minimal amount of DCM and adding this solution dropwise to a stirring solution containing diethyl ether and optionally hexanes and approximately 1-2 equivalents of HCl per mmol of Backbone Ester. The HCl was obtained from a commercially available solution of 2M HCl dissolved in diethyl ether. Alternatively, the 2M HCl was added to the combined fractions from the column purification prior to evaporation of solvent. Regardless, the solid crystalline product (of formula VI or VIb) was collected by vacuum filtration. This material could be stored for months in a refrigerator without any noticeable decomposition.

General Structure of Products Generated:

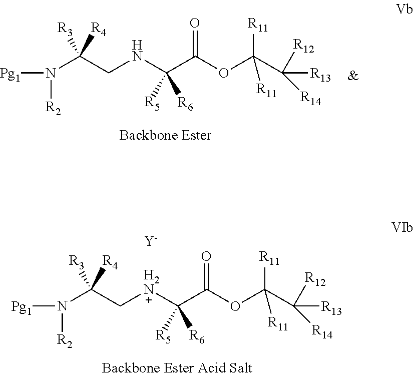

wherein, Y⁻, Pg₁, R₂, R₃, R₄, R₅, R₆, R₁₁, R₁₂, R₁₃ and R₁₄ are previously defined.

### Example 9B: Improved Reductive Amination Procedure

The disappointing yield of compound VIb-2 (Table 9B) led us to perform several small-scale reactions directed towards optimizing reaction yield. The following general procedure resulted from that optimization work.

The desired quantity of N-protected aldehyde (e.g. N-Fmoc-aminoacetaldehyde) was dissolved in a solution of denatured ethanol (Acros P/N 61105-0040; about 3-5 mL ethanol per mmol of N-protected aldehyde) and acetic acid (about 3 equivalents HOAc per mmol of N-protected aldehyde) at room temperature. Once all the solid dissolved, the solution was cooled in a salt/ice bath to about −15 to −5° C. To the cold stirring solution was added the amino acid ester salt (in a ratio of about 1.5 to 2 equivalents per mmol of aldehyde) and this solution stirred, preferably until the solid dissolved. To the cold stirring solution was added sodium cyanoborohydride NaBH₃CN) in a ratio of about 1.0 to 1.2 eq. of NaBH₃CN per mmol of aldehyde. As soon as practical after the addition of the NaBH₃CN, DI EA was optionally added dropwise to the reaction over 1-3 minutes in a ratio of about 0.8 to 1.0 eq. per mmol of amino acid ester salt used. When the reaction was deemed complete by TLC (usually in less than 1 hour), the ethanol was removed under reduced pressure and the residue was partitioned in EtOAc and deionized water. The product could be worked up essentially as described above in Example 9A except that an unsuccessful attempt was made to produce the HCl salt of the product prior to performing the column chromatography. However, for product VIb-2a as reported below, after column purification, to the combined column fractions was added 0.7 equivalents of p-toluene sulfonic acid-monohydrate (per mmol of starting aldehyde) and the solution was evaporated. To the oil residue was added 45 mL of ether and a small amount of EtOAc. A solid product crystallized on standing in a refrigerator overnight. The product was collected by vacuum filtration and washed with ether. ¹H-NMR analysis confirmed that this solid product was the tosyl salt of the Fmoc-aeg-OTBE backbone ester (Compound VIb-2a, in Table 9B, below).

### Example 9C: Preparation of Tosyl Salts of the Backbone Esters

Subsequently, in a reaction scaled to 3× the size of the reaction described in Example 9B (i.e. this reaction was run using 30 mmol N-Fmoc-aminoacetaldehyde), the reaction was performed as described and the ethanol was evaporated as described. However, at this point, the residue was partitioned with about 150 mL of EtOAc and 100 mL of water. The layers were separated and the EtOAc layer was washed one or more times with ½ saturated KH₂PO₄. CAUTION: These combined aqueous layers were then discarded to the waste stream for cyanide containing waste. To the ethyl acetate layer was added 75 mL of 1 N HCl (BEWARE gas evolution—which is likely HCN gas—perform in a properly certified hood with adequate ventilation). THIS AQUEOUS LAYER WAS NOT COMBINED WITH THE CYANIDE WASTE STREAM AS THAT WILL CAUSE HIGHLY TOXIC HCN GAS TO EVOLVE) The layers were separated and the EtOAc layer was immediately washed with 100 mL of saturated NaHCO₃. Because the pH of the wash was about 7 by paper, the ethyl acetate layer was then washed 1× with 100 mL of 5% NaHCO₃ and then once with about 100 mL of brine. The EtOAc layer was then dried over MgSO₄ (granular) and filtered. To the filtrate was added 23 mmol (0.76 eq per mmol of N-Fmoc-aminoacetaldehyde) of p-toluene sulfonic acid (monohydrate) and the solution was mixed until all the p-toluene sulfonic acid (monohydrate) dissolved. The product began to crystallize almost as soon as the p-toluene sulfonic acid (monohydrate) dissolved. The flask was allowed to stand at room temperature for 2-3 hours and then put in a refrigerator for several days. The solid product was collected by vacuum filtration and determined by 1H-NMR to be the tosyl salt the Fmoc-aeg-OTBE backbone ester (Compound VIb-2b in Table 9B, below). Accordingly, by this process, no column was needed to purify the material, which material was isolated in about 45% yield. This process was also successfully used to produce each of the chiral enantiomers of the tosyl salt of the gamma methyl Backbone Ester Acid Salt in good yield (as the TBE ester and the tosyl salt; Compounds VIb-5 and VIb-6 listed in Table 9B, below). In some cases, the tosyl salt was slow to crystallize so, in those cases, the solution in the recrystallization solvent could be evaporated and resuspended in a suitable solvent immediately before being used in a condensation reaction with a nucleobase acetic acid as described below.

TABLE 9B
Table of Products Generated (including examples to be produced)

| Cpd. # | Pg₁ | R₃ | R₄ | R₅ | R₆ | Acid Salt | Y⁻ | U | % Yield |
|---|---|---|---|---|---|---|---|---|---|
| VIb-1 | Fmoc | H | H | H | H | Yes | Cl⁻ | TCE | 43 |
| VIb-2 | Fmoc | H | H | H | H | Yes | Cl⁻ | TBE | 30 |
| VIb-2a | Fmoc | H | H | H | H | Yes | Ts- | TBE | 42 |
| VIb-2b | Fmoc | H | H | H | H | Yes | Ts- | TBE | 45 |
| VIb-3 | Fmoc | H | CH₃ | H | H | Yes | Cl⁻ | TCE | 28 |
| VIb-4 | Fmoc | H | CH₃ | H | H | Yes | Cl⁻ | TBE | 53 |
| VIb-5 | Fmoc | CH₃ | H | H | H | Yes | Ts⁻ | TBE | 51 |
| VIb-6 | Fmoc | H | CH₃ | H | H | Yes | Ts⁻ | TBE | 48 |
| VIb-7 | Fmoc | H | MP | H | H | Yes | Ts⁻ | TBE | — |
| VIb-8 | Fmoc | MP | H | H | H | Yes | Ts⁻ | TBE | — |
| VIb-9 | Fmoc | Ser | H | H | H | Yes | Ts⁻ | TBE | 64¹ |
| VIb-9b | Fmoc | Ser | H | H | H | Yes | Ts⁻ | 2IE | 62¹ |
| VIb-11 | Fmoc | H | Ser | H | Met | Yes | Ts⁻ | TBE | 51 |
| Vb-1 | Boc | H | H | H | H | No | N/A | TBE | 35² |

Legend to the Table:
Footnote 1: not isolated as a crystal;
Footnote 2: prepared using the method described by Feagin et. al. in Ref: C-31;
the abbreviation “Ser” refers to a protected serine side chain of formula: —CH₂—O—C(CH₃)₃.
Cl⁻ indicates the hydrochloride salt (i.e. HCl salt of the amine); Ts⁻ indicates the tosyl anion salt (i.e. Toluene sulfonic acid) of the protonated amine; U indicates the nature of the ester (e.g. either trichloroethyl (TCE); tribromoethyl (TBE) or 2-iodoethyl (2-IE).
The abbreviation “MP” refers to a miniPEG group of the formula —CH₂—(OCH₂CH₂)₂—O-^{t}Bu.

### Example 10: Synthesis of PNA Monomer Esters

Method 1: This method for preparation of PNA Monomer Esters is illustrated in FIG. 22, except that in all cases, the ‘Backbone Ester Acid Salt’ was used instead of the Backbone Ester because it is stable and can be stored and handled more easily. Nevertheless, the Backbone Ester can be used as a substitute if preferred by an individual user.

Generally, to the nucleobase acetic acid (in a ratio of about 1.0-1.3 equivalents as compared to the Backbone Ester Acid Salt to be used) was added dry ACN in a ratio of about 4-10 mL ACN per mmol of nucleobase acetic acid. This solution was cooled in an ice bath for 5-20 minutes and then about 2.5-6 eq. of NMM (with respect to the amount of nucleobase acetic acid used) was added. After stirring for 1-5 minutes, about 1.0-1.3 equivalents of TMAC was added and the reaction was allowed to stir for 20-30 minutes at 0° C. (Note: If the nucleobase does not comprise a protecting group (e.g. U or T), then the order of addition of NMM and TMAC was typically reversed). At this point, a sample was withdrawn and quenched by addition of a drop of the reaction mixture to a dilute solution of phenethylamine in ACN). TLC analysis (generally, 2-20% MeOH in DCM) of this quench was used to determine if the nucleobase acetic acid was completely converted to a mixed anhydride. If so, then the Backbone Ester Acid Salt (the limiting reagent) was added but if not, then additional TMAC was added until TLC revealed essentially complete conversion of the nucleobase acetic acid to a mixed anhydride. When sufficiently converted to a mixed anhydride, to the reaction was added the Backbone Ester Acid Salt and the reaction generally was allowed to proceed with stirring for about 30 minutes and then the ice bath was removed.

In some cases (e.g., when the nucleobase was difficult to solubilize in ACN), DMF was used instead of ACN (e.g. for the mono-Boc protected adenine and guanine nucleobases). In these cases, HBTU was used to activate the nucleobase acetic acid (instead of TMAC) and excess NMM was added as needed to maintain a basic pH). It was observed that several equivalents of HBTU was needed to completely activate the nucleobase acetic acid (as determined based on the phenethylamine quench result). Once properly activated, the nucleobase acetic acids were reacted by addition of the Backbone Ester Acid Salt.

The reaction was then allowed to warm to room temperature for 1-2 hours while being monitored by TLC. When complete, the ACN (or DMF as the case may be) was removed by evaporation under reduced pressure and the residue partitioned with EtOAc and one-half saturated KH₂PO₄. The layers were separated and the EtOAc layer was washed: (i) one or more times with one-half saturated KH₂PO₄, (ii) one or more times with 5% NaHCO₃, and (iii) one or more times with brine. The EtOAc layer was then dried with MgSO₄ (granular), filtered and evaporated. The residue (usually a foam) was then (unless it crystallized—See footnotes in Table 10B, below) purified by column chromatography using EtOAc/Hexanes (running an ethyl acetate gradient) or when the product was too polar, methanol/dichloromethane (running a MeOH gradient) was used. Both the hydrochloride and tosyl salts of the backbone ester were shown to be effective at producing the corresponding PNA Monomer Esters.

Method 2: This process was performed to determine how well the zinc reduction process would work on gamma miniPEG PNA monomer esters (which (in this case) possess a t-butyl ether moiety, in addition to the N-terminal Fmoc group and the Boc protection of the exocyclic amines of the nucleobases). For this process, Applicants took an impure sample of Compound 30-7 obtained from a commercial source as the starting material. The material was not suitable for PNA synthesis because a significant amount of the Boc group of the exocyclic amine had been removed (estimated to be 5-10%). To this sample of Compound 30-7 was added DCM in a ratio of about 4-5 mL per mmol of Compound 30-7. To the stirring solution was added about 1-1.05 equivalents of either 2,2,2-tribromoethanol (to produce Compound II-5) or 2-iodoethanol (to produce Compound II-7), about 0.1 equivalent of DMAP and about 1.05-1.1 equivalents of DCC. The solution was optionally cooled to 0° C. and was monitored by TLC. When the reaction appeared to complete by TLC, about 3-3.2 equivalents of di-t-butyl dicarbonate was added and the reaction was monitored by TLC. Curiously, no reaction with di-t-butyl dicarbonate was observed in TLC analysis of the sample containing 2-iodoethanol, but the sample containing the 2,2,2-tribromoethanol appeared to produce a new product. After stirring several hours, the reaction was quenched by the addition of water and then the DCU was removed by filtration. The filtrate was transferred to a separatory funnel and extracted: (i) once with one-half saturated KH₂PO₄, (ii) once with 5% NaHCO₃ and (iii) once with brine. The DCM layer was then dried over MgSO₄ (granular), filtered and evaporated. The residue was then purified by column chromatography using EtOAc/hexanes, running an EtOAc gradient. In some cases, the product was triturated by dissolving it in DCM and adding the DCM solution dropwise to a mixture of hexanes and ether. The triturated compound was collected by vacuum filtration.

General Structure of Products Generated:

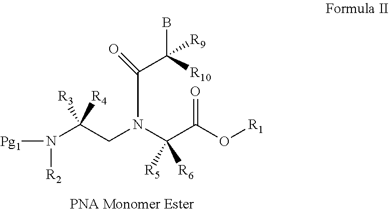

wherein, B, Pg₁, R₁, R₂, R₃, R₄, R₅, R₆, R₉ and R₁₀ are previously defined.

TABLE 10B
Table of Products Generated (including examles to be produced)

| Cpd. # | Pg₁ | R₃ | R₄ | R₅ | R₆ | B | B-Pg | Pos | Group/Atom | R₁ | Meth | % Yield |
|---|---|---|---|---|---|---|---|---|---|---|---|---|
| II-1 | Fmoc | H | H | H | H | C | Boc | 4 | ea | TCE | 1 | 71 |
| II-1-Ts | Fmoc | H | H | H | H | C | Boc | 4 | ea | TBE | 1 | 75⁴ |
| II-2 | Fmoc | H | CH₃ | H | H | C | Boc | 4 | ea | TCE | 1 | 70¹ |
| II-3 | Fmoc | H | CH₃ | H | H | C | Boc | 4 | ea | TBE | 1 | 60 |
| II-4 | Fmoc | H | CH₃ | H | H | C | Bis-Boc | 4 | ea | TBE | 1 | 58 |
| II-5 | Fmoc | H | MP | H | H | A | Bis-Boc | 6 | ea | TBE | 2 | 45 |
| II-6 | Fmoc | H | CH₃ | H | H | T | N/A | N/A | N/A | TCE | 1 | 49² |
| II-7 | Fmoc | H | MP | H | H | A | Boc | 6 | ea | 2-IE | 2 | 34 |
| II-8 | Fmoc | H | CH₃ | H | H | A | Bis-Boc | 6 | ea | TBE | 1 | 77 |
| II-9 | Fmoc | H | CH₃ | H | H | T | N/A | N/A | N/A | TBE | 1 | 54³ |
| II-10 | Fmoc | H | CH₃ | H | H | U^{2T} | Mob | 2 | S | TBE | 1 | 64 |
| II-11 | Fmoc | H | H | H | H | Y | N/A | N/A | N/A | TCE | 1 | 76 |
| II-12 | Fmoc | H | H | H | H | Y | N/A | N/A | N/A | TBE | 1 | 77 |
| II-12-Ts | Fmoc | H | H | H | H | Y | N/A | N/A | N/A | TBE | 1 | 75⁴ |
| II-13-Ts | Fmoc | H | H | H | H | T | N/A | N/A | N/A | t-Bu | 1 | 80+⁴ |
| II-14 | Fmoc | H | CH₃ | H | H | D | Bis-Boc | 2, 6 | ea | TBE | 1 | 88 |
| II-16-Ts | Fmoc | H | H | H | H | G | Boc | 2, 6 | ea | TBE | 1 | 55⁴ |
| II-17-Ts | Fmoc | H | H | H | H | A | Boc | 6 | ea | TBE | 1 | 68⁴ |
| II-18-Ts | Fmoc | H | H | H | H | D | Bis-Boc | 2, 6 | ea | TBE | 1 | 86⁴ |
| II-19-Ts | Fmoc | H | H | H | H | U^{2T} | Mob | 2 | S | TBE | 1 | 69⁴ |
| II-20-Ts | Fmoc | Ser | H | H | H | T | N/A | N/A | N/A | TBE | 1 | 56⁴ |
| II-21-Ts | Fmoc | Ser | H | H | H | C | Boc | 4 | ea | 2-IE | 1 | 61⁴ |
| II-22-Ts | Fmoc | Ser | H | H | H | A | Bis-Boc | 6 | ea | TBE | 1 | 11^{4, 5} |
| II-23-Ts | Fmoc | Ser | H | H | H | A | Bis-Boc | 6 | ea | 2-IE | 1 | 65⁴ |
| II-24-Ts | Fmoc | H | MP | H | H | T | N/A | N/A | N/A | TBE | 1 | 63⁴ |

Legend to the Table;
In all cases, R₉ and R₁₀ are H.
Footnote 1: Very insoluble product - recrystallized from 2/2/1 EtOH/ACN/H₂O.
Footnote 2: Product recrystallized from EtOH.
Footnote 3: Product recrystallized from EtOAc/Hexanes.
Footnote 4: prepared from the tosyl salt (instead of the hydrochloride salt) of the backbone ester. In all cases R₂ is H; R₉ is H and R₁₀ is H.
Footnote 5: Activation of the nucleobase with HBTU proved troublesome in this case leading to a lower than typical yield.
The abbreviation “MP” refers to a miniPEG group of the formula —CH—(OC₂CH₂)₂—O-^{t}Bu.
The abbreviation “Ser” refers to a protected serine side chain of formula: —CH₂—O—C(CH₃)₃.
The abbreviation “Met” refers t the metionine side chain of formula: —CH₂CH₂—S—CH₃.
The column entitled “B-Pg” identifies the nucleobase protecting group (Pg).
The column entitled “Pos” identifies the position of the nucleobase ring to which the nucleobase protecting group is linked.
The column entitled “Group/Atom” identifies the atom or group to which the protecting group is linked.
The symbol “ea” identifies the group as an exocyclic amine.
The column entitled “R₁” identifies the ester type of the PNA Monomer Ester (e.g. TCE = 2,2,2-trichloroethyl, TBE = 2,2,2-tribromoethyl and 2-IE = 2-iodoethyl).
The column entitled “Meth” identifies the method used to prepare the PND Monomer Ester.
B refers to the nucleobase wherein nucleobases and protecting groups are attached to the compound of formula II as illustrated in FIGS. 18B.

### Example 11: Zinc-Based Reduction of PNA Monomer Esters to PNA Monomers

Method 1: The general process for reduction of PNA Monomer Esters to PNA Monomers is illustrated in FIG. 23. According to some embodiments of the method, to the PNA Monomer Ester was added THF (in a ratio of about 5-12 mL per mmol of PNA Monomer Ester). This solution was then cooled in an ice bath for about 10-30 minutes. To the ice cold stirring solution was added about one-half to one equivalent volume of ice cold TXE Buffer [TXE Buffer was made by combining (or in similar ratios) 50 mmol KH₂PO₄, 25 mmol of ethylenediaminetetraacetic acid (EDTA) and 25 mmol of ethylenediaminetetraacetic acid zinc disodium salt hydrate (EDTA-Zn.H₂O) in about 150 mL to 250 mL of deionized water and about 50 mL to 85 mL of glacial acetic acid. This mixture was permitted to stir overnight after which about 100 mL to 200 mL of THF was added and after about 30-60 minutes of additional stirring, the solids were removed by filtration and the resulting filtrate was used as TXE Buffer] and zinc dust (about 5 to 10 eq. based on the PNA Monomer Ester). If solubility of the PNA Monomer Ester was an issue or otherwise deemed prudent, additional THF, saturated KH₂PO₄, water and/or acetic acid was added. As the reaction proceeded, saturated KH₂PO₄ solution (and optionally water) was added and additional zinc dust was added until the reaction appeared complete by TLC analysis (10-20% MeOH in DCM). When deemed complete, the reaction mixture was then filtered through celite to remove the zinc and other insoluble material. Generally, the filtrate was then reduced in volume under reduced pressure until the solution began to freeze (form a slushy composition) on the rotary evaporator (no heat added to the flask). DCM or EtOAc, water and/or Extraction Buffer was then added to partition the product into the DCM or EtOAc (Extraction Buffer was prepared as: 1 g KH₂PO₄ and 0.5 g KHSO₄ per 10 mL of deionized water). In some cases the aqueous layer could be back extracted one or more times with additional DCM or EtOAc, as appropriate. The (combined) organic layer(s) (DCM or EtOAc) was/were washed one or more times (often 3×) with the Extraction Buffer and then one or more times with saturated NaCl (brine). The organic layer was then dried over MgSO₄ (granular), filtered, and evaporated. The crude product was then optionally dissolved in a minimum of DCM and precipitated by dropwise addition to a briskly stirring solution of hexanes or hexanes/diethyl ether (generally in a ratio of about 1/1 to 8/2), except that Compound 30-5 (Table 11B) required a mixture of hexanes and di-n-butyl ether to form a precipitate. The precipitated product could be (and preferably was) allowed to stir for 1-2 hours before being collected by vacuum filtration, but in any case, was collected by vacuum filtration and dried under high vacuum. The PNA Monomer was then used in some cases in PNA oligomer synthesis without further purification or was optionally purified by column chromatography on silica gel (generally in DCM/MeOH running a methanol gradient). If the material was to be purified by column chromatography, the precipitation was generally not performed until after the column purification was performed. After column chromatography, the PNA Monomer was often precipitated as described above to obtain material in a form suitable for handling and weighing.

Method 2: According to some embodiments of the method, to the PNA Monomer Ester was added THF (in a ratio of about 5-12 mL per mmol of PNA Monomer Ester). This solution was then cooled in an ice bath (or salt/ice bath) for 10-15 minutes. To the ice cold stirring solution was then added an equivalent volume of TXE Buffer and generally, this mix was allowed to cool for several minutes before proceeding. Zinc dust (about 10 eq. based on the PNA Monomer Ester) was then added, usually in ⅓ increments along with acetic acid (0.5-2 mL per mmol PNA Monomer Ester), ice cold saturated KH₂PO₄ (0.5-2 mL per mmol PNA Monomer Ester), and ice-cold water (0.5-2 mL per mmol PNA Monomer Ester), each at about 15-30 minute intervals (for TBE esters but longer intervals for TCE esters) until all the zinc was added. If solubility of the PNA Monomer Ester was an issue, additional THF, water or glacial acetic acid was added as needed to solubilize the PNA Monomer Ester. Additional zinc dust was added as needed to drive the reaction to completion. The reaction was monitored by TLC analysis (10-20% MeOH in DCM) and allowed to stir until complete. For the TBE esters (and 2-IE esters), that was generally 1-2 hours, unless the starting material exhibited limited solubility. For TCE esters, the reaction was significantly slower (3-6 hours unless the PNA Monomer Ester exhibited limited solubility)—which was observed to significantly extend the reaction time) and really never went to completion (usually >80%)). When deemed complete, the reaction mixture was then filtered through celite to remove the zinc and other insoluble material and worked up as described under Method 1, above.

Methods 1 and 2 are an adaptation of the procedure described by Just et al. (Ref. C-14). Applicants observed that performing the reactions at 0° C. and in the presence of acetic acid (which pushed the pH of the reaction below 4.2 and is not described by Just) resulted in highly specific removal of the TCE, TBE and 2-IE protecting groups generally without any significant removal of (or reaction with) other protecting groups such as Fmoc, ^{t}Bu, Boc, Bis-Boc, or Mob (sulfur protection). In Applicants' hands, the TBE esters were the most labile, followed by the 2-IE esters with the TCE esters being the least labile (i.e. most difficult to remove). In Applicants' hands, the TBE esters were found to be extremely soluble and easiest to work with. However, an exceedingly pure PNA monomer was produced with the 2-IE ester (see Table 11B, Compound 30-21, Footnote 9). Methods 1 & 2 were varied for some starting materials to improve upon conditions or to account for differing reactivities. Such variations are considered routine experimentation.

PNA Monomers that were prepared were generally examined by ¹H-NMR and exhibited spectra consistent with the expected product. PNA Monomers (i.e. 30-3 and 30-5 to 30-10 and 30-12 in precipitated but not column-purified form) were successfully used in standard synthesis protocols to prepare PNA oligomers of the expected mass. The impurity profiles of these PNA oligomers so produced were generally not significantly different from those made with other commercially available PNA Monomer used in our laboratories. Column purified monomers made from this process generally produced improved purity and yields of PNA oligomer (as compared with commercially available materials).

Certain of the Chiral PNA Monomers were also examined for chiral purity by their use in the preparation of a 6-mer oligomer of the sequence: SEQ ID No: 1: L-Phe-X-gly-gly-gly-gly, wherein X is the PNA Monomer to be examined for chiral purity. The L-enantiomer of phenylalanine (L-Phe) was used because it is relatively hydrophobic and can be obtained in near 100% optical purity. A four residue C-terminal (gly)₄ tail was used to add enough length to isolate the oligomer product by conventional methods. By substituting the chiral Phe molecule (i.e. the X-PNA Monomer) in the oligomer, a diastereomer is created by any chiral impurity (opposite enantiomer) of the X-PNA Monomer. In our experience, the diastereomers of the 6-mer oligomers of this structure are well resolved by standard HPLC protocols. By this test, all chiral PNA Monomers tested were found to have greater than 90% enantiomeric excess (ee), often exceeding 95% optical purity. Compound 30-24 was confirmed to exceed 99% optical purity and several other compounds are, based on this analysis, believed to exceed 99% optical purity.

Chiral PNA Monomers 30-3, 30-8 and 30-9 were used to prepare a 12-mer PNA oligomer of nucleobase sequence (SEQ ID No. 2) CCCTAACCCTAA. The purified 12-mer PNA oligomer was then examined in thermal melting experiments and found to exhibit various expected functional properties of a chiral gamma substituted PNA oligomer. For example, this PNA oligomer made from gamma methyl substituted PNA Monomers had essentially the same Tm (under identical conditions) as a PNA oligomer of identical nucleobase sequence made from gamma miniPEG substituted PNA Monomers.

Taken together, this data demonstrated that the procedures described herein can be used to prepare PNA Monomer Esters of a great diversity of structure (including chirally pure materials) and that these PNA Monomer Esters can be converted in high yield to PNA Monomers suitable for use in standard PNA oligomer synthesis protocols. It is noteworthy that no column purification was required of these PNA Monomers prior to their use in oligomer synthesis—but ultimately was desirable to produce very high quality PNA oligomers. In some embodiments, simple extraction and precipitation was performed to put the PNA Monomers in condition for use in oligomer synthesis.

Method 3 (t-butyl ester removal—applied to produce Compound 30-13): To the PNA Monomer Ester (tBu ester) was added dichloromethane (about 2 mL per mmol of PNA Monomer Ester). This solution was cooled in an ice bath and then trifluoroacetic acid (TFA—about 2 mL per mmol of PNA Monomer Ester) was added and the reaction proceeded in the ice bath. TLC analysis (10% MeOH/DCM) indicated a very slow reaction so the ice bath was removed and the reaction warmed to room temperature. After about 7 hrs., the solvent was removed under reduced pressure and the residue was co-evaporated once from acetonitrile. The product was then dissolved in acetonitrile (about 4 mL per mmol SM) and allowed to crystallize out upon standing overnight in a refrigerator. The solid product was collected by vacuum filtration.

General Structure of Products Generated:

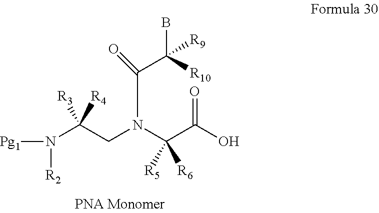

wherein, B, Pg₁, R₂, R₃, R₄, R₅, R₆, R₉ and R₁₀ are previously defined.

TABLE 11B
Table of Products Generated (including examples to be produced)

| Cpd. # | Pg₁ | R₃ | R₄ | R₅ | R₆ | B | B-Pg | Pos | Group/Atom | Ester SM | Meth | % Yield |
|---|---|---|---|---|---|---|---|---|---|---|---|---|
| 30-1 | Fmoc | H | H | H | H | C | Boc | 4 | ea | TCE | 2 | 100¹ |
| 30-2 | Fmoc | H | H | H | H | C | Boc | 4 | ea | TBE | 2 | 80⁵ |
| 30-3 | Fmoc | H | CH₃ | H | H | C | Boc | 4 | ea | TBE | 2 | 83 |
| 30-4 | Fmoc | H | CH₃ | H | H | C | Bis-Boc | 4 | ea | TBE | 2 | 0² |
| 30-5 | Fmoc | H | MP | H | H | A | Bis-Boc | 6 | ea | TBE | 1 | 61 |
| 30-6 | Fmoc | H | CH₃ | H | H | T | N/A | N/A | N/A | TCE | 1 | 54 |
| 30-7 | Fmoc | H | MP | H | H | A | Boc | 6 | ea | 2-IE | 1 | 73 |
| 30-8 | Fmoc | H | CH₃ | H | H | A | Bis-Boc | 6 | ea | TBE | 1 | 85 |
| 30-9 | Fmoc | H | CH₃ | H | H | T | N/A | N/A | N/A | TBE | 1 | 68 |
| 30-10 | Fmoc | H | CH₃ | H | H | U^{2T} | Mob | 2 | S | TBE | 2 | 76 |
| 30-11 | Fmoc | H | H | H | H | Y | N/A | N/A | N/A | TCE | 2 | 75³ |
| 30-12 | Fmoc | H | H | H | H | Y | N/A | N/A | N/A | TBE | 2 | 35^{4, 5} |
| 30-13 | Fmoc | H | H | H | H | T | N/A | N/A | N/A | t-Bu | 3 | 95⁶ |
| 30-14 | Fmoc | H | CH₃ | H | H | D | Bis-Boc | 2,6 | ea | TBE | 2 | 80 |
| 30-16 | Fmoc | H | H | H | H | G | Boc | 2 | ea | TBE | 2 | 66⁶ |
| 30-17 | Fmoc | H | H | H | H | A | Boc | 6 | ea | TBE | 2 | 65⁵ |
| 30-18 | Fmoc | H | H | H | H | D | Bis-Boc | 2,6 | ea | TBE | 2 | 63⁵ |
| 30-18 | Fmoc | H | H | H | H | D | Bis-Boc | 2,6 | ea | TBE | 2 | 92⁶ |
| 30-19 | Fmoc | H | H | H | H | U^{2T} | Mob | 2 | S | TBE | 2 | 59⁶ |
| 30-20 | Fmoc | Ser | H | H | H | T | N/A | N/A | N/A | TBE | 2 | 67^{5, 7} |
| 30-21 | Fmoc | Ser | H | H | H | C | Boc | 4 | ea | 2-IE | 2 | 75^{5, 9} |
| 30-22 | Fmoc | Ser | H | H | H | A | Bis-Boc | 6 | ea | 2-IE | 2 | 23⁵ |
| 30-23 | Fmoc | H | Ser | Met | H | T | N/A | N/A | N/A | TBE | 2 | 64⁵ |
| 30-23b | Fmoc | H | Ser | Met | H | C | Boc | 4 | ea | TBE | 2 | 40⁵ |
| 30-24 | Fmoc | H | MP | H | H | T | N/A | N/A | N/A | TBE | 2 | 74^{5, 8} |

Legend to the Table: In all cases, R₉ and R₁₀ are H.
Footnote 1: crude yield - scale was too small to workup;
Footnote 2: Applicants determined that the 5-6 double bond of the cytosine nucleobase is significantly reduced under these conditions if the exocyclic amine protecting group is Bis-Boc, whereas no significant reduction of the 5-6 double bond was observed under these conditions if the protection group of the exocyclic amine is mono-Boc (compare Compounds 30-3 & 30-4).
Footnote 3; For comparison, when the traditional LiOH saponification of this PNA Monomer Ester was performed, an 18% yield of the product was obtained; This PNA Monomer made by the traditional saponification method however did not contain any contaminate “ene” caused by reduction of the ‘yne’ whereas the product compound 30-11 contained about 10-15% contaminating ‘ene’;
Footnote 4; This material did not appear to contain any ‘ene’ contaminate.
Footnote 5: Reported yield is for column purified material.
Footnote 6: Obtained as a crystal. In all cases R₂ is H; R₉ is H and R₁₀ is H.
Footnote 7: Enantiomeric purity estimated to be greater than 99% based on LCMS analysis (but subject to confirmation once authentic samples of the other enantiomer is prepared).
Footnote 8: Enantiomeric purity determined to be greater than 99% based on LCMS analysis and comparison to authentic samples comprising the other enantiomer.
Footnote 9: Isolated purity of this column purified monomer was determined to exceed 99.5% by HPLC analysis at 260 nm.
The abbreviation “Ser” refers to a protected serine side chain of formula: —CH₂—O—C(CH₃)₃.
The abbreviation “Met” refers to the methionine side chain of formula: —CH₂CH₂—S—CH₃.
The abbreviation “MP” refers to a miniPEG group of the formula —CH₂—(OCH₂CH₂)₂—O-^{t}Bu.
The column entitled “B-Pg” identifies the nucleobase protecting group (Pg).
The column entitled “Pos” identifies the position of the nucleobase ring to which the protecting group is linked.
The column entitled “Group/Atom” identifies the atom or group of the nucleobase to which the protecting group is linked.
The symbol “ea” identifies the group as the exocyclic amine.
The column entitled “Ester SM” identifies the type of ester of the PNA Monomer Ester (TCE = 2,2,2-trichloroethyl, TBE = 2,2,2-tribromoethyl and 2-IE = 2-iodoethyl used as starting material for preparation of the PNA Monomer (as its free carboxylic acid).
The column entitled “Meth” identifies the method used to prepare the PNA Monomer from the PNA Monomer Ester.
B refers to the nucleobase wherein nucleobases and protecting groups are attached to the compound of formula 30 as illustrated in FIGS. b.

### Example 12: Reduction of Fmoc-γ-L-ala-(Bis-Boc-C)-OTBE Monomer Ester (Compound II-4) Using Tri-n-butylphosphine (TBP)

Because of the potential for unwanted side reductions as noted in Footnotes 2 to 4 of Table 11B, alternative reducing agents and related procedures were investigated. One possible alternative was to apply the transacylation methodology described by Hans et al. (Ref. C-7)) to potentially produce a free acid instead. In this example, Fmoc-γ-L-ala-(Bis-Boc-C)-OTBE PNA Monomer Ester (Compound 11-4-10.5 mg, 10.8 μmol) was dissolved in 210 μL of N,N′-dimethyl formamide (DMF). Aliquots of 50 μL of this stock solution were combined with water, N,N′-dimethyl-4-aminopyridine (DMAP), and N-methylmorpholine (NMM), and then treated lastly with tri-n-butyl-phosphine (TBP) as follows:

| | | Water | DMAP | NMM | TBP |
|---|---|---|---|---|---|
| Sample No. | Temperature | (5 uL) | (2 mg) | (2 uL) | (2 uL) |
| 1 | −41° C. | + | − | − | + |
| 2 | −41° C. | − | − | − | + |
| 3 | −41° C. | + | + | + | + |
| 4 | RT | − | − | − | + |

Reactions were equilibrated to the indicated temperature prior to addition of TBP and then maintained at the indicated temperature for 30 min whereupon about 1 μL of the reaction mixture was diluted with about 0.5 mL of acetonitrile. The acetonitrile mixture (about 10 μL) was analyzed by reversed-phase HPLC (C18 column, 5-95% acetonitrile linear gradient into 0.1% aqueous formic acid over 15 minutes). The HPLC system employed was equipped with a diode array detector and a mass detector (LC-MS) allowing simultaneous monitoring of UV absorbance and compound mass (M+H). Results of the analyses are shown in FIGS. 24a and 24b. M+H values for the brominated compounds are reported as the largest isotopic peak observed in the mass spectrum. Mass accuracy of the system was +/−˜0.5-0.75 Da.

The data indicate that the Fmoc-γ-L-ala-(Bis-Boc-C)-OTBE PNA Monomer Ester (Compound II-4) was cleanly deprotected in DMF at −41 C and RT within 30 minutes, whereas reactions which contained water led to appreciable amounts of the di-bromoethyl ester of the monomer (See: Ref. C-7)). Also noteworthy, no reduction of the 5-6 double bond of the cytosine heterocycle was detected as compared with the zinc, acetic acid and buffered phosphate conditions under which this 5-6 double bond was appreciably reduced (Footnote 2 in Table 11B) when bis-Boc protected—but not when mono-Boc protected.

### Example 13: Reduction of Fmoc-γ-L-ala-(Bis-Boc-A)-OTBE Monomer Ester (Compound II-8) Using Tri-n-butylphosphine (TBP)

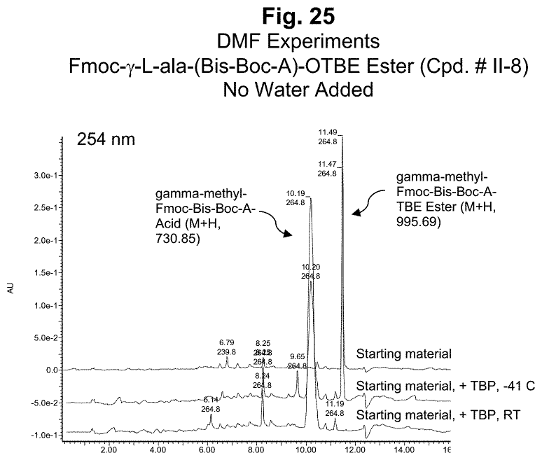

Following the procedures outlined above, the reduction of Fmoc-γ-L-ala-(Bis-Boc-A)-OTBE PNA Monomer Ester was tested in DMF at RT and −41° C. Reactions of 2.5 mg of monomer ester (Cpd. #II-8, 2.5 μmol) in 50 μL were treated with 2 μL of TBP. The results of these experiments are shown in FIG. 25.

The data indicate that Fmoc-γ-L-ala-(Bis-Boc-A)-OTBE PNA Monomer Ester (Cpd. #II-8) is only partially deprotected within 30 minutes at −41° C. whereas it is completely and cleanly deprotected within 30 minutes in DMF at room temperature.

### Example 14: Reduction Using TBP in Tetrahydrofuran (THF) as Compared to DMF

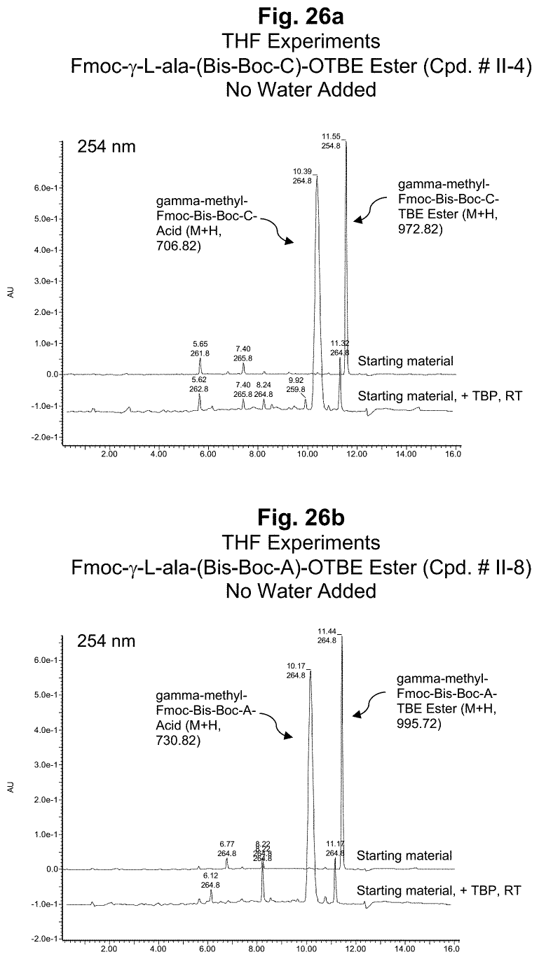

Following the procedures outlined above, the reduction of Fmoc-γ-L-ala-(Bis-Boc-C)-OTBE PNA Monomer Ester (Compound II-4) and Fmoc-γ-L-ala-(Bis-Boc-A)-OTBE PNA Monomer Ester (Compound II-8) were tested in THF at RT. The results are shown in FIGS. 26a & 26b.

The data indicate that both compounds are fully reduced yielding a majority of PNA Monomer and 10-15% of the respective dibromoethyl ester. The dibromoethyl esters of the C and A monomers have retentions of 11.32 and 11.17 minutes in the Figures, respectively. For ease of reaction work-up, THF may be a preferred solvent due to its higher volatility than the much higher boiling DMF.

### Example 15: Synthesis of N-Fmoc-N-Boc-Ethylenediamine (Compound 75)

To a 3-neck round bottomed flask equipped with a mechanical stirrer was added Fmoc-O Su and acetone (in a ratio of about 1.2 mL acetone per mmol of Fmoc-O-Su). To this stirring solution was added dropwise a mixture of N-Boc-ethylenediamine (in a ratio of about 1.1 mmol N-Boc-ethylenediamine per mmol of Fmoc-O Su) dissolved in acetone (in a ratio of about 0.72 mL of acetone per mmol of N-Boc-ethylenediamine) over 30 minutes. Then a mixture of NaHCO₃ (in a ratio of about one mmol NaHCO₃ per mmol of Fmoc-O-Su), Na₂CO₃ (in a ratio of about 0.5 mmol Na₂CO₃ per mmol of Fmoc-O-Su) and water (in a ratio of about 1.5 mL water per eq. of Fmoc-O-Su) was added dropwise over 30 minutes. The reaction was allowed to stir an additional 30 minutes and monitored by TLC (in 5% MeOH/DCM). Then 1N HCl was added dropwise to the reaction (in a ratio of about 2.2 eq. HCl per mmol of Fmoc-O-Su). After addition, the pH of the solution was in the range of 2-3 (by paper) and could be adjusted if needed by addition of more acid or base as necessary. The white solid was filtered off and the filter cake was washed well with a solution of 35/65 acetone/water. The filter cake was then washed well with neat acetonitrile to remove water and placed under high vacuum until dry. For this reaction, 200 mmol of Fmoc-O-Su produced 189 mmol of product (95% yield). Product (compound 75) was confirmed by ¹H-NMR.

### Example 16: Synthesis of N-Fmoc-ethylenediamine—Acid Salt (Compound 53a)

Example 16a: Synthesis of TFA salt (Compound 53a-TFA): To compound 75 (SM) was added DCM (in a ratio of about 1 mL DCM per mmol of SM) and this solution was placed in an ice bath with stirring. The solution was allowed to stir for 5 minutes while cooling and then TFA (in a ratio of about 1 mL TFA per mmol of SM) was added slowly. The reaction was allowed to stir for 45 minutes and monitored by TLC (in 5% MeOH/DCM). When TLC indicated the reaction was complete, the solution was then filtered through silica, and the filtrate was concentrated to yellow oil. Optionally, the yellow oil could be subject to azeotropic distillation with toluene to remove excess TFA. To the yellow oil was then added diethyl ether (in a ratio of about 3.3 mL diethyl ether per mmol of SM) and let stir for 1 hour. The solid product was collected by filtration, washed with diethyl ether and placed under high vacuum until dry. Additional crops of product could be obtained by concentration of the mother liquor.

| mmol Starting Material | mmol of Product | |
|---|---|---|
| (SM) | (53b-TFA) | % Yield |
| 89.3 | 73 | 82.4 |
| 58.7 | 51 | 87.7 |

Example 16b: Synthesis of HCl Salt (Compound 53a-HCl): The TFA Salt (Compound 53a-TFA) was dissolved in EtOAc (in a ratio of about 1.3 mL EtOAc per mmol of 53a-TFA). To this stirring solution was added 1N HCl (aqueous) slowly (in a ratio of about 3 eq. HCl per mmol of 53a-TFA). This was allowed to stir for 10 minutes, then the product was collected by filtration, washed with water, and placed under high vacuum until dry.

| mmol 53a-TFA | mmol of Product | % Yield |
|---|---|---|
| 75 | 58 | 78 |

### Example 17: Synthesis of bromoacetate esters (Compounds 52)

This procedure is generally adapted from Seuring and Seebach (Ref C-34). Generally, to an oven-dried round bottom flask equipped with an oven-dried addition funnel placed under N₂ was added bromoacetyl bromide and THF (in a ratio of about 1.6 mL THF per mmol of bromoacetyl bromide). The round bottom flask was placed in an ice bath with stirring for 15 minutes to cool. In an oven-dried Erlenmeyer flask was combined the alcohol of choice (in a ratio of about 1 mmol alcohol per mmol of bromoacetyl bromide), pyridine (in a ratio of about 1 mmol pyridine per mmol of bromacetyl bromide), and THF (in a ratio of about 0.2 to 0.4 mL per mmol of bromoacetyl bromide). If the alcohol is a liquid, then no additional THF is necessary. This mixture was then placed in the oven-dried addition funnel and added dropwise over about 20 minutes. The ice bath was removed and the reaction was allowed to stir for about 30 minutes while warming to room temperature and monitored by TLC (in 25/75 EtOAc/Hexanes). When complete by TLC, the reaction mixture was vacuum filtered to remove the solid and the filtrate was concentrated to an oil. The crude reaction product was purified by column chromatograph on silica gel running ethyl acetate/hexanes for elution. Table 17 provides a list of products and yields obtained.

TABLE 17

| | mmol Starting | mmol of | |
|---|---|---|---|
| Alcohol Used | Material (SM) | Product | % Yield |
| Allyl Alcohol | 300 | 248 | 83 |
| Tribromoethanol | 150 | 106 | 71.2 |
| Trichloroethanol | 200 | 164 | 82 |
| Bromoethanol | 150 | 48.6 | 55.3 |

### Example 18: Synthesis of Backbone Esters (Compounds 54 and 54a) and Their Conversion to Tosyl Salts (Compounds 55 & 55a)

To Compound 53a-TFA (SM) was added ethanol (in a ratio of about 4 mL ethanol per mmol of SM) and toluene (in a ratio of about 2 mL toluene per mmol of SM). This was evaporated, and then toluene was added (in a ratio of about 2 mL toluene per mmol of SM) and evaporated again. This was placed in the high vacuum for 30 minutes to dry. Then the desired bromoacetate ester (See Table 18—Compound 52a) was added (in a ratio of about 1.4 mmol bromoacetate ester per mmol of SM) and the reaction was placed under N₂. Then dry acetonitrile was added (in a ratio of about 6.5 mL ACN per mmol of SM) and the reaction was placed in an ice bath. This was allowed to stir for about 5 minutes while cooling and then DIEA was added (in a ratio of about 2.7 mmol DIEA per mmol of SM) via an addition funnel over about 5 minutes. The ice bath was removed and the reaction was allowed to stir for about 45 minutes while being monitored by TLC (in 5% MeOH/DCM). Once TLC indicated the reaction was complete (about 1 hr.), 1N HCl was added (in a ratio of about 1.2 eq. HCl per mmol of SM). After the addition, the pH was in the range 4-5 (by paper). The reaction was then concentrated to about ⅓ of its volume, and to the residue was added EtOAc (in a ratio of about 7.5 mL EtOAc per mmol of SM) and extracted 1× with H₂O, 3× with 3.33% aqueous citric acid, 1× H₂O, 2× saturated NaHCO₃, 1× 5% NaHCO₃ and finally 1× with brine (saturated NaCl). The organic layer was dried over MgSO₄ (granular) and then optionally filtered through a minimum of silica gel (i.e. a “mini column”), using ethyl acetate as the eluent in a volume sufficient to elute all UV-active material from the column. To the eluent was then added p-toluenesulfonic acid (in a ratio of about 0.7 mmol TSA per mmol of SM). The flask was agitated until the p-toluenesulfonic acid was dissolved and the product then crystallized from the solution. After standing for some time, the solution was placed in a refrigerator to finish crystallizing. Crystals of the product were collected by vacuum filtration and washed using cold EtOAc. Surprisingly, crystals of tosyl salts obtained from crude reaction products were very clean and did not generally need to be recrystallized before being used to produce PNA Monomer Esters.

TABLE 18

| Bromoacetate | mmol Starting | mmol of | |
|---|---|---|---|
| Ester (52) | Material (SM) | Product | % Yield |
| allyl bromoacetate | 10 | 4.5 (5.7) | 45 (57)¹ |
| 2,2,2-tribromoethyl bromoacetate | 30 | 14 | 47.3 |
| 2,2,2-tribromoethyl bromoacetate | 6.87 | 3.88 | 56.5 |
| t-butyl bromoacetate² | 21.5 | 11 | 51.5 |
| 2-bromoethyl bromoacetate | 57.1 | 24.1 | 42.2 |

Numbers in parentheses in Table 18 represent yield prior to recrystallization. Footnote 1: No “mini column” was run; crude product was concentrated under reduced pressure after addition of p-toluenesulfonic acid and then precipitated by stirring briskly in a mixture of diethyl ether and a minimum amount of ethyl acetate for a few hours. The product was then recrystallized from ethyl acetate. Numbers in parenthesis in Table 18 represent yield prior to recrystallization. Footnote 2: t-butyl bromoacetate was obtained from a commercial source.

### 7. References

### US Patent Literature

| Ref. | | |
|---|---|---|
| No. | Citation | Authors, Title and Dates |
| A-1 | U.S. Pat. No. | Nielsen, P. E., Buchardt, O., Berg, R. H., |
| | 6,107,470 | Egholm, M., “Histidine-containing peptide |
| | | nucleic acids”, Aug. 22, 2000 |
| A-2 | U.S. Pat. No. | Coull, J. M., Egholm, M., Hodge, R. P., Ismail, |
| | 6,133,444 | M., Rajur S. B., “Synthons For The Synthesis |
| | | And Deprotection Of Peptide Nucleic Acids |
| | | Under Mild Conditions”, Oct. 17, 2000 |
| A-3 | U.S. Pat. No. | Coull, J. M., Egholm, M., Hodge, R. P., Ismail, |
| | 6,172,226 | M., Rajur S. B., “Synthons For The Synthesis |
| | | And Deprotection Of Peptide Nucleic Acids |
| | | Under Mild Conditions”, Jan. 9, 2001 |
| A-4 | U.S. Pat. No. | Gildea, B. D., Coull, J. M., “PNA Synthons”, |
| | 6,265,559 | Jul. 24, 2001 |
| A-5 | U.S. Pat. No. | Ly, D., Rapireddy, S., Sahu, B., |
| | 9,193,759 | “Conformationally-Preorganized, MiniPEG- |
| | | Containing Gamma-Peptide Nucleic Acids”, |
| | | Nov. 24, 2015 |

### Foreign Patent Literature

| Ref. | | |
|---|---|---|
| No. | Citation | Authors, Title and Dates |
| B-1 | WO92/20702 | Buchardt, O., Egholm, M., Nielsen, P. E., Berg, |
| | | R. H., “Peptide Nucleic Acids”; May 22, 1992 |
| B-2 | WO92/20703 | Buchardt, O., Egholm, M., Nielsen, P. E., Berg, |
| | | R. H., “The use of nucleic acid analogues in |
| | | Diagnostics and Analytical Procedures”; May 22, |
| | | 1992 |
| B-3 | WO95/17403 | Coull, J. M., Hodge, R. P., “Guanine Synthons |
| | | For Peptide Nucleic Acid Synthesis and Methods |
| | | For Production” Jun. 29, 1995 |
| B-4 | WO96/40709 | Gildea, B. D., Coull, J. M., “PNA-DNA |
| | | Chimeras and PNA Synthons For Their |
| | | Preparation”; May 29, 1996 |
| B-5 | WO12/138955 | Ly, D., Rapireddy, S., Sahu, B., |
| | | “Conformationally-Preorganized, MiniPEG- |
| | | Containing Gamma-Peptide Nucleic Acids”, Oct. |
| | | 11, 2012 |

### Scientific Literature References

| Ref. No. | Citation |
|---|---|
| C-1 | Abiko, A., Masamune, S., “An Improved, Convenient Procedure for |
| | Reduction of Amino Acids to Aminoalcohols: Use of NaBH₄—H₂SO₄” Tett. |
| | Lett. 33(38): 5517-5518 (1992) |
| C-2 | Adamiak, R. W., Biata, E., Grzeskowiak, K., Kierzek, R., Kraszewski, A., |
| | Markiewicz, W. T., Stawinski, J., Wiewiorowski, M., “Nucleotide 3′- |
| | phosphotriesters as key intermediates for the oligoribonucleotide synthesis. |
| | IV. New method of removal of 2,2,2-trichloroethyl group and ³¹P NMR as a |
| | new tool for analysis of deblocking of internucleotide phosphate protecting |
| | groups”; NAR, 4(7): 2321-2330 (1977) |
| C-3 | Babu, V. V. S., Sudarshan, K., Sudarshan, N. S., “Synthesis of Fmoc- |
| | protected β-amino alcohols and peptidyl alcohols from Fmoc-amino |
| | acid/peptide acid azides”; Indian Journal of Chemistry, 45B: 1880-1886 |
| | (2006) |
| C-4 | Cook, Alan, “The Use of β,β,β-Tribromoethyl Chloroformate for the |
| | Protection of Nucleoside Hydroxyl Groups”; JOC, 33(9): 3589-3593 (1968) |
| C-5 | Falorni, M., Porcheddu, A., Taddei, M., “Mild Reduction of Carboxylic Acids |
| | to Alcohols Using Cyanuric Chloride and Sodium Borohydride”; Tett. Lett., |
| | 40: 4395-4396 (1999) |
| C-6 | Gansäuer, A., Dahmen, T., “Reductive Cleavage of 2,2,2-Trichloroethyl |
| | Esters by Titanocene Catalysis”; CHIMIA, 66: 433-434 (2012) |
| C-7 | Hans, J. J., Driver, R. W., Burke, S. D., “Direct Transacylation of 2,2,2- |
| | Trihaloethyl Esters with Amine and Alcohol Using Phosphorus (III) |
| | Reagents for Reductive Fragmentation and in Situ Activation”; JOC, 65: |
| | 2114-2121 (2000) |
| C-8 | Huang, H., Joe, G. H., Choi, S. R., Kim, S. N., Kim, Y. T., Pak, C. S., Hong, |
| | J. H., Lee, W., “Synthesis of Enantiopure γ-Glutamic Acid Functionalized |
| | Peptide Nucleic Acid Monomers”, Bull. Korean Chem. Soc., 31(7): 2054- |
| | 2056 (2010) |
| C-9 | Huang, H., Joe, G. H., Choi, S. R., Kim, S. N., Kim, Y. T., Pak, H. S., Kim, |
| | S. K., Hong, J. H., Han, H-K., Kang, J. S., Lee, W., “Preparation and |
| | Determination of Optical Purity of γ-Lysine Modified Peptide Nucleic Acid |
| | Analogues”; Arch Pharm Res, 35(3): 517-522 (2012) |
| C-10 | Huber, D. P., “Catalytic Enantioselective Synthesis of α-Fluoro α-Amino |
| | Acid Derivatives” Thesis - Doctor of Natural Sciences, Swiss Federal |
| | Institution of Technology Zurich (1977) |
| C-11 | Isidro-Llobet, A., Alvarez, M., Albericio, F., “Amino Acid-Protecting Groups”; |
| | Chem., Rev., 109: 2455-2504 (2009) |
| C-12 | Ivkovic, J., Lembacher-Fadum, C., Breinbauer, R., “A rapid and efficient |
| | one-pot method for the reduction of N-protected α-amino acids to chiral α- |
| | amino aldehydes using CDI/DIBAL-H”; Organic & Biomolecular Chemistry, |
| | 13: 10456-10460 (2015) |
| C-13 | Iyer, R. P., Nucleobase Protection of Deoxyribo- and Ribonucleotides; |
| | Current Protocols in Nucleic Acid Chemistry, 2.1.1-2.1.17 (2000) |
| C-14 | Just. G., Grozinger, K., A Selective, “Mild Cleavage of Trichloroethyl Esters, |
| | Carbamates, and Carbonates to Carboxylic Acids, Amines, and Phenol |
| | using Zinc/Tetrahydrofuran/pH 4.2-7.2 Buffer”; Synthesis, 7: 457-458 |
| | (1976) |
| C-15 | Kokotos, G., “A convenient One-Pot Conversion of N-Protected Amino |
| | Acids and Peptides into Alcohols”; Synthesis Papers, 299 (April 1990) |
| C-16 | Marinier, B., Kim, Y. C., Navarre, J-M, “The 2,2,2-Trichloroethyl Group for |
| | Carboxyl Protection During Peptide Synthesis”; Can. J. Chem., 51: 208-214 |
| | (1973) |
| C-17 | Meyer, S. D., Schreiber, S. L., “Acceleration of the Dess-Martin Oxidation by |
| | Water”; JOC, 59: 7549-7552 (1994) |
| C-18 | Myers, A. G., Zhong, B., Movassaghi, M., Kung, D. W., Lanman, B. A., Kwon, |
| | S., “Synthesis of highly epimerizable N-protected α-amino aldehydes of |
| | high enantiomeric excess”; Tett. Lett., 41: 1359-1362 (2000) |
| C-19 | Olsen, R. K., Apparao, S., Bhat, K. L., “Synthesis of a Model Analogue of the |
| | Cyclic Decapeptide Intercalating Agent Luzopeptin A (Antibiotic BBM 928A) |
| | Containing Proline, Valine and Unsubstituted Quinoline Substituents”; JOC, |
| | 51(16): 3079-3085 (1986) |
| C-20 | Ramesh, D., Anand & Vimal, “A Convenient and Mild Procedure for the |
| | Reduction of Amino Acids Using Amberlyst 15 - NaBH₄—LiCl”; Tett. Lett. |
| | 39: 917-918 (1998) |
| C-21 | Rodriguez, M., Llinares, M., Doulut, S., Heitz, A., Martinez, J., “A Facile |
| | Synthesis of Chiral N-Protected α-Amino Alcohols”; Tett. Lett., 32(7): 923- |
| | 926 (1991) |
| C-22 | Salvi, J-P., Walchshofer, N., Paris, J., “Formation of Bis (Fmoc-amino |
| | ethyl)-N-glycine derivatives by reductive amination of Fmoc-amino |
| | aldehydes with NaBH₃CN”; Tett. Lett., 35(8): 1181-1184 (1994) |
| C-23 | Somsak, L., Czifrak, K., Veres, E., “Selective removal of 2,2,2- |
| | trichloroethyl- and 2,2,2-trichloroethoxycarbonyl protecting groups with Zn—N- |
| | methylimidazole in the presence of reducible and acid-sensitive |
| | functionalities”; Tett. Lett., 45: 9095-9097 (2004) |
| C-24 | Sureshbabu, V. V., Sudarshan, N. S., Chennakrishnareddy, G., “Simple and |
| | rapid synthesis of N^{α}-urethane protected β-amino alcohols and peptide |
| | alcohols employing HATU”: Indian Journal of Chemistry, 48B: 574-579 |
| | (2009) |
| C-25 | Vellemäe, E.: Lebedev, O.: Sillard, R.: Mäeorg, U., “A selective method for |
| | cleavage of N-Troc-protected hydrazines and amines under mild conditions |
| | using mischmetal and TMSCI”, J. Chem. Res., 11: 685-687 (2006) |
| C-26 | Wen, J. J., Crews, C. M., “Synthesis of 9-fluorenylmethoxycarbonyl- |
| | protected amino aldehydes”, Tetrahedron: Asymmetry, 1998, 9: 1855-1858 |
| C-27 | Wojciechowski, F., Hudson, R. H. E., “A Convenient Route to N-[2- |
| | (Fmoc)aminoethyl]glycine Esters and PNA Oligomerization Using a Bis-N- |
| | Boc Nucleobase Protecting Group Strategy”; JOC, 73: 3807-3816 (2008) |
| C-28 | Woodward, R. B., Heusler, K., Gosteli, J., Naegeli, P., Oppolzer, W., |
| | Ramage, R., Ranganathan, S., Vorbüggen, H., “The Total Synthesis of |
| | Cephalosporin C”; JACS, 88(4): 852-853 (1966) |
| C-29 | Zhang, J., Fu, J., Si, W., Wang, X., Wang, Z., Tang, J., “A highly efficient |
| | deprotection of the 2,2,2-trichloroethyl group at the anomeric oxygen of |
| | carbohydrates”; Carbohydrate Research, 346: 2290-2293 (2011) |
| C-30 | Chen, J. J., Aduda, V., “DMSO-Aided o-lodoxybenzoic Acid (IBX) oxidation |
| | of Fmoc-Protected Amino Alcohols”, Synthetic Communications, 37: 3493- |
| | 34999 (2007) |
| C-31 | Feagin, T. A., Shah, N. I., Heemstra, J. M., “Convenient and Scalable |
| | Synthesis of Fmoc-Protected Peptide Nucleic Acid Backbone”, Journal of |
| | Nucleic Acids, Article ID 354549 (2012) |
| C-32 | Kakarla, R., Liu, J., Nadughambi, D., Chang, W., Mosley, R. T., Bao, D., |
| | Micolochick Steuer, H. M., Keilman, M., Bansal, S., Lam, A. M., Seibel, W., |
| | Neilson, S., Furman, P. A., Sofia, M. J., “Discovery of a Novel Class of |
| | Potent HCV NS4B Inhibitors: SAR Studies on Piperizinone Derivatives”, |
| | J. Med. Chem., 57: 2136-2160 (2014) |
| C-33 | SciFinder Search of Aldehydes of Fmoc Amino Acids, 15 pages, 74 |
| | structures (2017) |
| C-34 | Seuring and Seebach, Justus Liebigs Annalen der Chemie, 12: 2066 |
| | (1978) |
| C-35 | Wu, Y., Xu, J-C., “Synthesis of chiral peptide nucleic acids using Fmoc |
| | chemistry”, Tetrahedron, 57: 8107-8113 (2001) |
| C-36 | Novosjolova, I, Kennedy, S., Rozners, E., 2-Methoxypyridine as a |
| | Thymidine Mimic in Watson-Crick Base Pairs of DNA and PNA: Synthesis, |
| | Thermal Stability, and NMR Structural Studies, ChemBioChem, 18: 1-7 |
| | (2017) |

While the present teachings are described in conjunction with various embodiments, it is not intended that the present teachings be limited to such embodiments. On the contrary, the present teachings encompass various alternatives, modifications and equivalents, as will be appreciated by those of skill in the art.

I(We) claim:

1. A compound of formula VI: 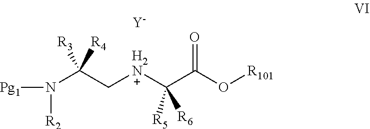
wherein: Y⁻ is a sulfonate anion;
Pg₁ is an amine protecting group;
R₁₀₁ is a branched or straight chain C₁-C₄ alkyl group or a group of formula I; 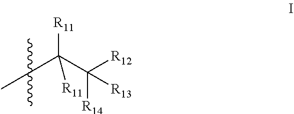
wherein, each R₁₁ is independently H, D, F, C₁-C₆ alkyl, C₃-C₆ cycloalkyl or aryl;
each of R₁₂, R₁₃ and R₁₄ is independently selected from the group consisting of:
H, D, F, Cl, Br and I, provided however that at least one of R₁₂, R₁₃ and R₁₄ is selected from Cl, Br and I;
R₂ is H, D or C₁-C₄ alkyl;
each of R₃, R₄, R₅, and R₆ is independently selected from the group consisting of:
H, D, F, and a side chain selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIy, IIIw, IIIx, IIIy, IIIz, IIIaa, and IIIab, wherein each of IIIi, IIIj, IIIk, IIIm, IIIn, IIIo, IIIp, IIIq, IIIr, IIIs, IIIt, IIIu, IIIy, IIIw, IIIx, IIIy and IIIz optionally comprises a protecting group; 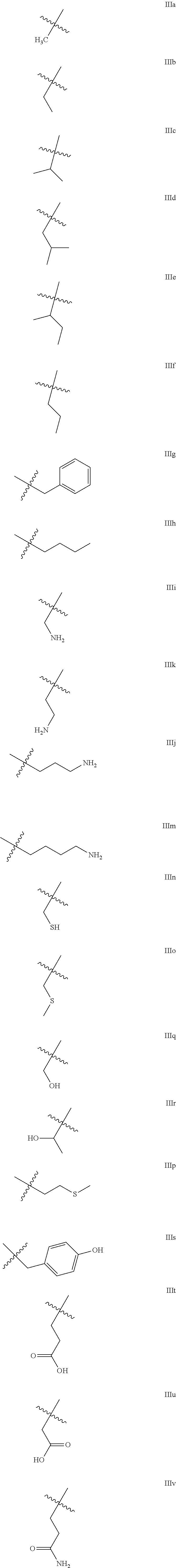 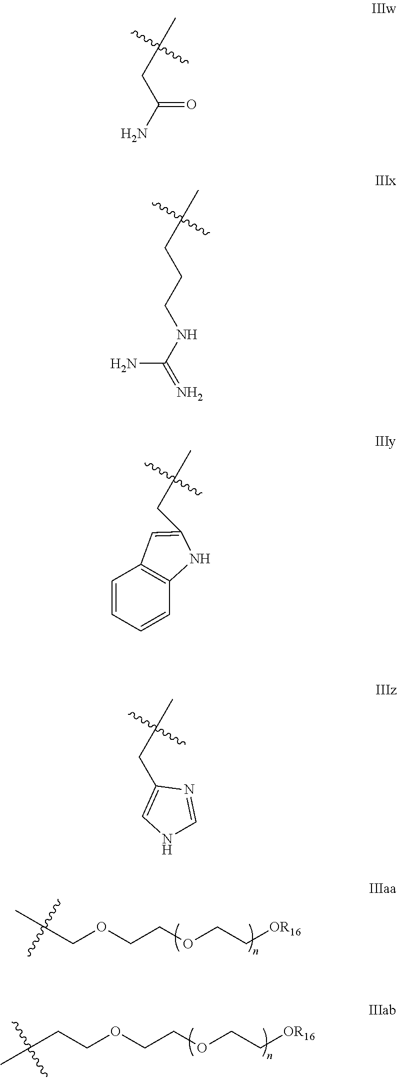
wherein, R₁₆ is selected from H, D and C₁-C₄ alkyl group; and
n is a number from 0 to 10, inclusive.

2. The compound of claim 1, wherein the sulfonate anion is produced from a sulfonic acid selected from the group consisting of: benzenesulfonic acid, naphthalenesulfonic acid, p-xylene-2-sulfonic acid, 2,4,5-trichlorobenzenesulfonic acid, 2,6-dimethylbenzenesulfonic acid, 2-mesitylenesulfonic acid, 2-mesitylenesulfonic acid dihydrate, 2-methylbenzene sulfonic acid, 2-ethylbenzenesulfonic acid, 2-isopropylbenzenesulfonic acid, 2,3-dimethylbenzenesulfonic acid, 2,4,6-trimethylbenzenesulfonic acid and 2,4,6-triisopropylbenzenesulfonic acid.

3. The compound of claim 1, wherein the sulfonate anion is produced from p-toluenesulfonic acid.

4. The compound of claim 1, wherein Y⁻ is selected from benzenesulfonate, p-toluenesulfonate, naphthalenesulfonate, p-xylene-2-sulfonate, 2,4,5-trichlorobenzenesulfonate, 2,6-dimethylbenzenesulfonate, 2-mesitylenesulfonate, 2-mesitylenesulfonate dihydrate, 2-methylbenzene sulfonate, 2-ethylbenzenesulfonate, 2-isopropylbenzenesulfonate, 2,3-dimethylbenzenesulfonate, 2,4,6-trimethylbenzenesulfonate, and 2,4,6-triisopropyl benzenesulfonate.

5. The compound of claim 1, Y⁻ is p-toluenesulfonate.

6. The compound of claim 1, wherein at least one of R₃ and R₄ is independently selected from the group of formulas IIIaa and IIIab.

7. The compound of claim 6, wherein R₁₆ is H, D, methyl or t-butyl, and n is 1, 2, 3 or 4.

8. The compound of claim 1, wherein R₂ is H or D.

9. The compound of claim 6, wherein R₂ is H, R₁₆ is methyl or t-butyl and n is 1 or 2.

10. The compound of claim 1, wherein each of R₅ and R₆ is independently H, D or F.

11. The compound of claim 1, wherein Pg₁ is selected from the group consisting of: Nsc, Bsmoc, Nsmoc, ivDde, Fmoc*, Fmoc(2F), mio-Fmoc, dio-Fmoc, TCP, Pms, Esc, Sps and Cyoc.

12. The compound of claim 1, wherein Pg₁ is Fmoc.

13. The compound of claim 1, wherein Pg₁ is selected from the group consisting of: Trt, Ddz, Bpoc, Nps, Bhoc, Dmbhoc and Floc.

14. The compound of claim 1, wherein Pg₁ is Boc.

15. The compound of claim 1, wherein R₁₀₁ is 2,2,2-trichloroethyl, 2,2,2-tribromoethyl, 2-iodoethyl or 2-bromoethyl.

16. The compound of claim 1;
wherein one of R₃, R₄, R₅ and R₆ is independently selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIy, IIIw, IIIx, IIIy, IIIz, IIIaa, and IIIab, wherein each of IIIi, IIIj, IIIk, IIIm, IIIn, IIIo, IIIp, IIIq, IIIr, IIIs, IIIt, IIIu, IIIy, IIIw, IIIx, IIIy and IIIz optionally comprises a protecting group; and
the others of R₃, R₄, R₅ and R₆ are independently H, D or F.

17. The compound of claim 1;
wherein each of R₅ and R₆ is independently H, D or F;
one of R₃ and R₄ is independently selected from the group consisting of: IIIa, IIIb, IIIc, IIId, IIIe, IIIf, IIIg, IIIh, IIIj, IIIk, IIIm, IIIn, IIIo, IIIp, IIIq, IIIr, IIIs, IIIt, IIIu, IIIy, IIIw, IIIx, IIIy, IIIz, IIIaa, and IIIab, wherein each of IIIi, IIIj, IIIk, IIIm, IIIn, IIIo, IIIp, IIIq, IIIr, IIIs, IIIt, IIIu, IIIy, IIIw, IIIx, IIIy and IIIz optionally comprises a protecting group; and
the other of R₃ and R₄ is H, D or F.

18. The compound of claim 1, wherein one of R₃ or R₄ is a group of formula IIIaa or IIIab: 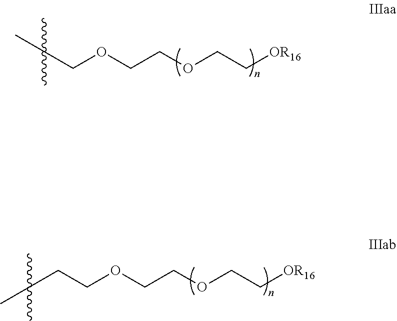
and the other of R₃ and R₄ is H, wherein, n is 0, 1, 2, 3 or 4 and R₁₆ is H, methyl or t-butyl.

19. The compound of claim 16, wherein Pg₁ is selected from the group consisting of: Nsc, Bsmoc, Nsmoc, ivDde, Fmoc*, Fmoc(2F), mio-Fmoc, dio-Fmoc, TCP, Pms, Esc, Sps and Cyoc.

20. The compound of claim 16, wherein Pg₁ is Fmoc.

21. The compound of claim 16, wherein Pg₁ is selected from the group consisting of: Trt, Ddz, Bpoc, Nps, Bhoc, Dmbhoc and Floc.

22. The compound of claim 16, wherein Pg₁ is Boc.

23. The compound of claim 16, wherein the sulfonate anion is p-toluenesulfonate.

24. The compound of claim 16, wherein R₁₀₁ is 2,2,2-trichloroethyl-, 2,2,2-tribromoethyl-, 2-iodoethyl- or 2-bromoethyl.

25. A kit comprising: a) a compound according to claim 1; and b) (i) instructions; (ii) a base acetic acid; and/or (iii) a solvent.

26. A compound of formula VI-T: 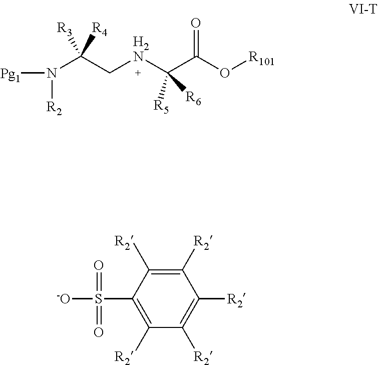
wherein, Pg₁ is an amine protecting group;
R₁₀₁ is selected from the group consisting of: methyl, ethyl, n-propyl, isopropyl, n-butyl, iso-butyl, sec-butyl, tert-butyl, allyl, 2-iodoethyl, 2-bromoethyl, 2,2,2-trichloroethyl, 2,2,2-trifluoroethyl, 2,2,2-tribromoethyl and tert-butyldimethylsilyl;
R₂ is H, D or C₁-C₄ alkyl;
each R₂′ is independently H, D, F, Cl, Br, I or C₁-C₄ alkyl; and
each of R₃, R₄, R₅, and R₆ is independently selected from the group consisting of: H, D, F, and a side chain selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIy, IIIw, IIIx, IIIy, IIIz, IIIaa, and IIIab, wherein each of IIIi, IIIj, IIIk, IIIm, IIIn, IIIo, IIIp, IIIq, IIIr, IIIs, IIIt, IIIu, IIIy, IIIw, IIIx, IIIy and IIIz optionally comprises a protecting group; 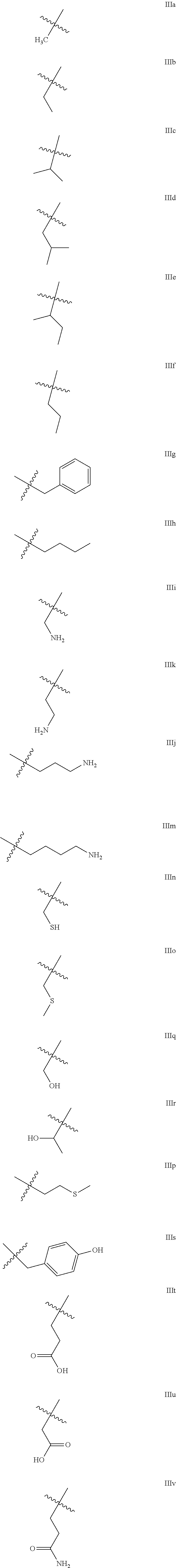 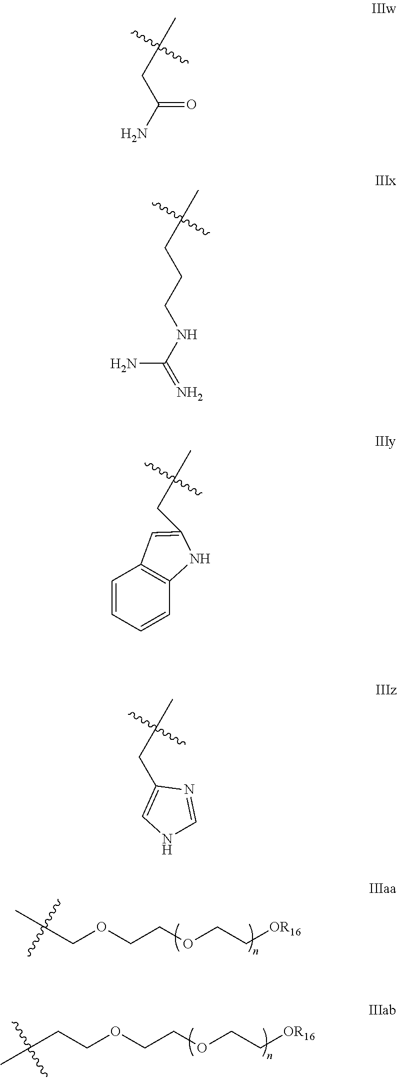
wherein, R₁₆ is selected from H, D and C₁-C₄ alkyl group; and
n is a number from 0 to 10, inclusive.

27. A compound of formula VI-Ts: 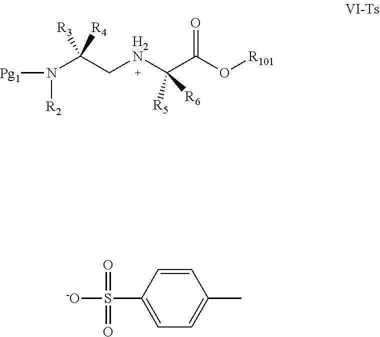
wherein, Pg₁ is an amine protecting group;
R₁₀₁ is selected from the group consisting of: methyl, ethyl, n-propyl, isopropyl, n-butyl, iso-butyl, sec-butyl, tert-butyl, allyl, 2-iodoethyl, 2-bromoethyl, 2,2,2-trichloroethyl, 2,2,2-trifluoroethyl, 2,2,2-tribromoethyl and tert-butyldimethylsilyl;
R₂ is H, D or C₁-C₄ alkyl; and
each of R₃, R₄, R₅, and R₆ is independently selected from the group consisting of: H, D, F, and a side chain selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIy, IIIw, IIIx, IIIy, IIIz, IIIaa, and IIIab, wherein each of IIIi, IIIj, IIIk, IIIm, IIIn, IIIo, IIIp, IIIq, IIIr, IIIs, IIIt, IIIu, IIIy, IIIw, IIIx, IIIy and IIIz optionally comprises a protecting group; 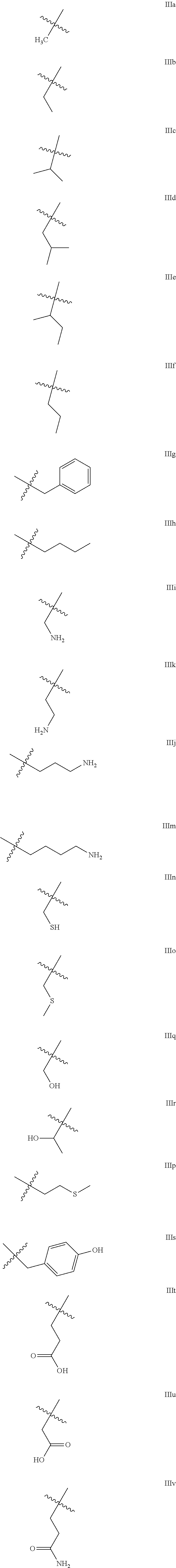 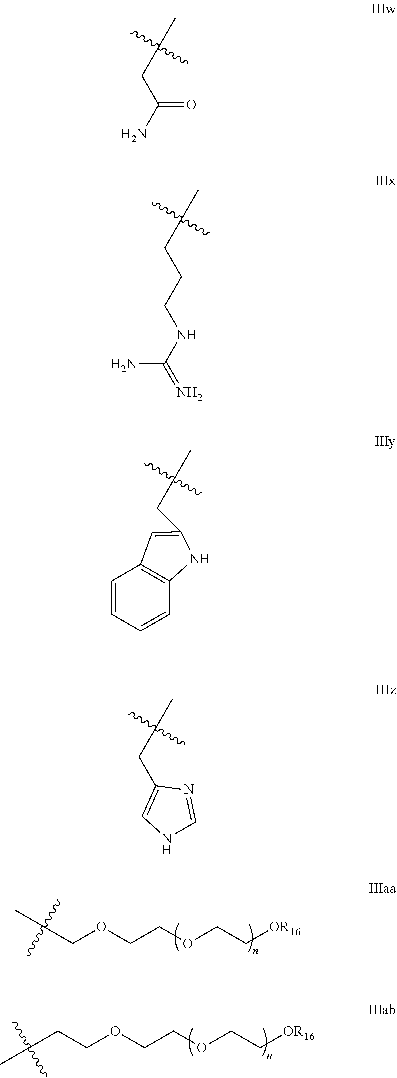
wherein, R₁₆ is selected from H, D and C₁-C₄ alkyl group; and
n is a number from 0 to 10, inclusive.

28. The compound of claim 26, wherein at least one of R₃ and R₄ is independently selected from the group consisting of formulas IIIaa and IIIab.

29. The compound of claim 28, wherein R₁₆ is H, D, methyl, or t-butyl, and n is 1, 2, 3 or 4.

30. The compound of claim 26, wherein R₂ is H or D.

31. The compound of claim 26, wherein R₂ is H, R₁₆ is methyl or t-butyl, and n is 1 or 2.

32. The compound of claim 26, wherein each of R₅ and R₆ is independently H, D, or F.

33. The compound of claim 26, wherein Pg₁ is selected from the group consisting of: Nsc, Bsmoc, Nsmoc, ivDde, Fmoc*, Fmoc(2F), mio-Fmoc, dio-Fmoc, TCP, Pms, Esc, Sps and Cyoc.

34. The compound of claim 26, wherein Pg₁ is Fmoc.

35. The compound of claim 26, wherein Pg₁ is selected from the group consisting of: Trt, Ddz, Bpoc, Nps, Bhoc, Dmbhoc and Floc.

36. The compound of claim 26, wherein Pg₁ is Boc.

37. The compound of claim 26, wherein R₁₀₁ is selected from 2,2,2-trichloroethyl, 2,2,2-tribromoethyl, 2-iodoethyl and 2-bromoethyl.

38. The compound of claim 26;
wherein one of R₃, R₄, R₅ and R₆ is independently selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIy, IIIw, IIIx, IIIy, IIIz, IIIaa, and IIIab, wherein each of IIIi, IIIj, IIIk, IIIm, IIIn, IIIo, IIIp, IIIq, IIIr, IIIs, IIIt, IIIu, IIIv, IIIw, IIIx, IIIy and IIIz optionally comprises a protecting group; and
the others of R₃, R₄, R₅ and R₆ are independently H, D or F.

39. The compound of claim 26;
wherein each of R₅ and R₆ is independently H, D or F;
one of R₃ and R₄ is independently selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIy, IIIw, IIIx, IIIy, IIIz, IIIaa, and IIIab, wherein each of IIIi, IIIj, IIIk, IIIm, IIIn, IIIo, IIIp, IIIq, IIIr, IIIs, IIIt, IIIu, IIIy, IIIw, IIIx, IIIy and IIIz optionally comprises a protecting group; and
the other of R₃ and R₄ is H, D or F.

40. The compound of claim 26, wherein one of R₃ or R₄ is a group of formula IIIaa or IIIab: 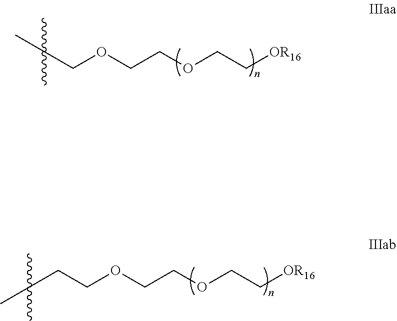
and the other of R₃ and R₄ is H, wherein, n is 0, 1, 2, 3 or 4 and R₁₆ is methyl or t-butyl.

41. The compound of claim 38, wherein Pg₁ is selected from the group consisting of: Nsc, Bsmoc, Nsmoc, ivDde, Fmoc*, Fmoc(2F), mio-Fmoc, dio-Fmoc, TCP, Pms, Esc, Sps and Cyoc.

42. The compound of claim 38, wherein Pg₁ is Fmoc.

43. The compound of claim 38, wherein Pg₁ is selected from the group consisting of: Trt, Ddz, Bpoc, Nps, Bhoc, Dmbhoc and Floc.

44. The compound of claim 38, wherein Pg₁ is Boc.

45. The compound of claim 38, wherein the sulfonate anion is produced from p-toluenesulfonic acid.

46. The compound of claim 38, wherein R₁₀₁ is 2,2,2-trichloroethyl, 2,2,2-tribromoethyl, 2-iodoethyl or 2-bromoethyl.

47. The compound of claim 26, wherein each R₃, R₄, R₅ and R₆ is independently H, D or F.

48. The compound of claim 26, wherein Pg₁ is Fmoc, R₂ is H, and each of R₃, R₄, R₅ and R₆ is H.

49. The compound of claim 26, wherein Pg₁ is Boc, R₂ is H, and each of R₃, R₄, R₅ and R₆ is H.

50. The compound of claim 26, wherein R₁₀₁ is methyl, ethyl, tert-butyl, allyl, or tert-butyldimethylsilyl.

51. The compound of claim 26, wherein R₁₀₁ is 2-iodoethyl, 2-bromoethyl, 2,2,2-trichloroethyl, or 2,2,2-tribromoethyl.

52. A compound of formula VI-Ts-A:

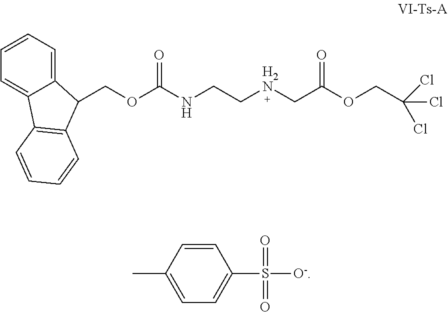

53. A compound of formula VI-Ts-B:

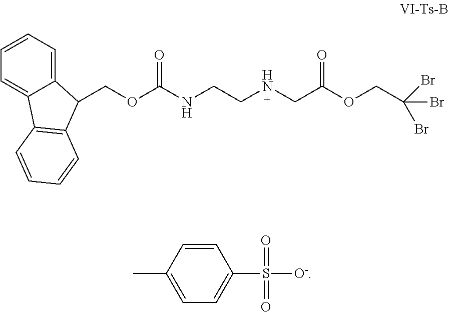

54. A compound of formula VI-Ts-C:

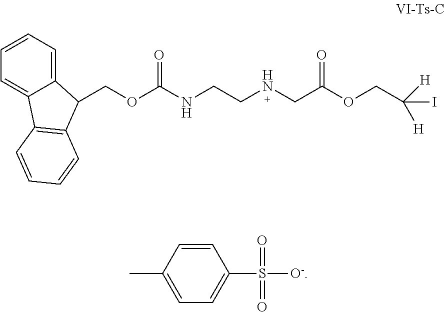

55. A compound of formula VI-Ts-D:

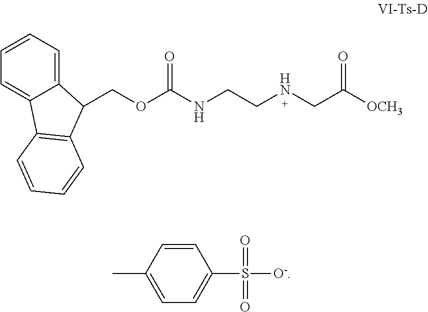

56. A compound of formula VI-Ts-E:

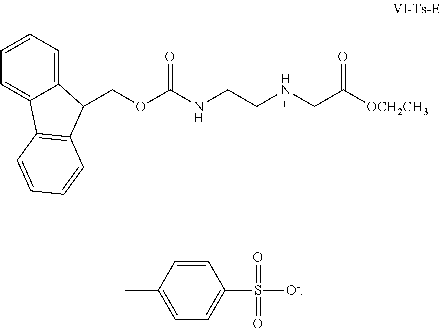

57. A compound of formula VI-Ts-F:

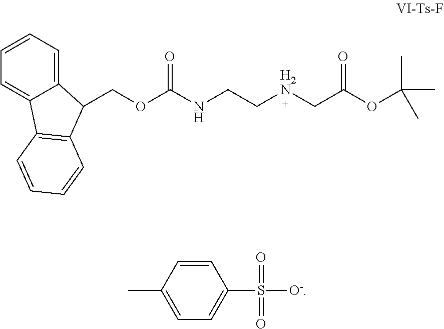

58. A compound of formula VI-Ts-G:

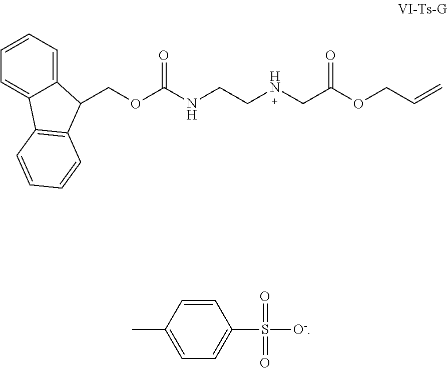

59. A compound of formula VI-Ts-H: 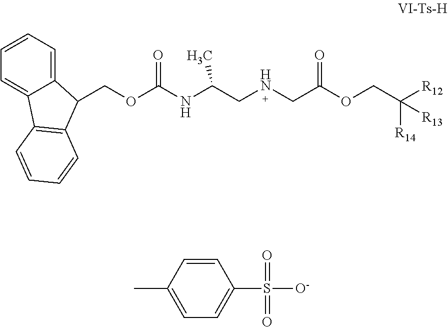
wherein, each of R₁₂, R₁₃ and R₁₄ is independently H, D, F, Cl, Br or I, provided however that at least one of R₁₂, R₁₃ and R₁₄ is selected from Cl, Br and I.

60. A compound of formula VI-Ts-I: 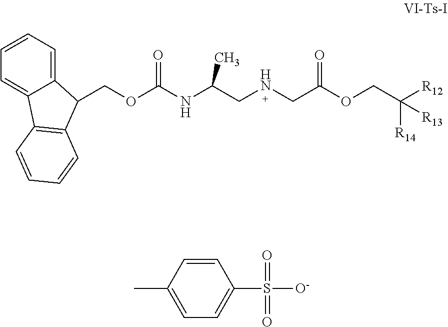
wherein, each of R₁₂, R₁₃ and R₁₄ is independently H, D, F, Cl, Br or I, provided however that at least one of R₁₂, R₁₃ and R₁₄ is selected from Cl, Br and I.

61. A compound of formula VI-Ts-J: 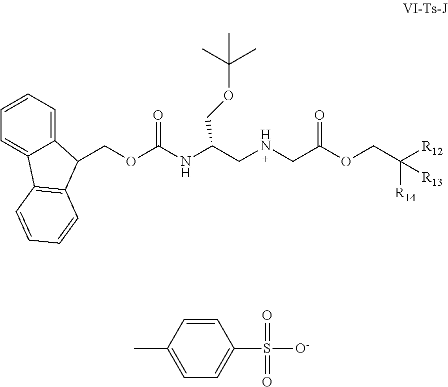
wherein, each of R₁₂, R₁₃ and R₁₄ is independently H, D, F, Cl, Br or I, provided however that at least one of R₁₂, R₁₃ and R₁₄ is selected from Cl, Br and I.

62. A compound of formula VI-Ts-K: 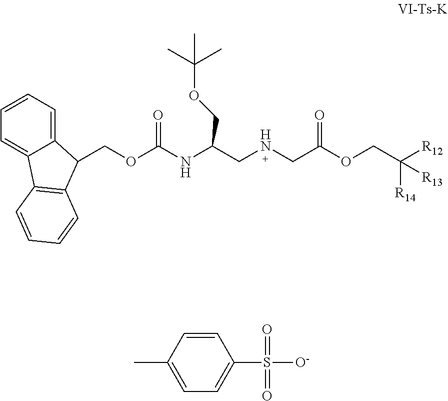
wherein, each of R₁₂, R₁₃ and R₁₄ is independently H, D, F, Cl, Br or I, provided however that at least one of R₁₂, R₁₃ and R₁₄ is selected from Cl, Br and I.

63. A compound of formula VI-Ts-L:

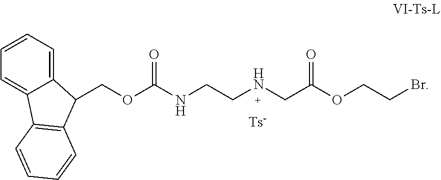 [SMILES: O=C(CNCCNC(=O)OCC1c2ccccc2-c2ccccc21)OCCBr]

64. A method comprising:
(i) reacting a compound of formula 53a: 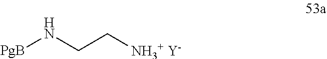
with a compound of formula 52a: 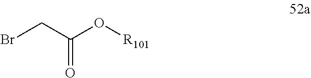
wherein PgB is a base-labile amine protecting group; R₁₀₁ is a branched or straight chain C₁-C₄ alkyl group or a group of formula I; 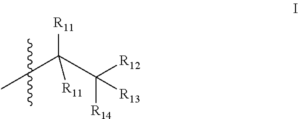
wherein,
each R₁₁ is independently H, D, F, C₁-C₆ alkyl, C₃-C₆ cycloalkyl or aryl;
each of R₁₂, R₁₃ and R₁₄ is independently H, D, F, Cl, Br or I, provided however that at least one of R₁₂, R₁₃ and R₁₄ is selected from Cl, Br and I; and
Y⁻ is an anion;
(ii) wherein the reaction proceeds in the presence of a tertiary base and wherein the reaction produces a product of formula 54a:

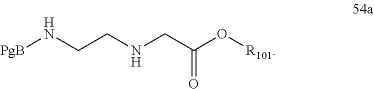

65. The method of claim 64, further comprising: contacting the compound of formula 54a with at least one equivalent of a sulfonic acid to thereby produce a compound of formula 55a: 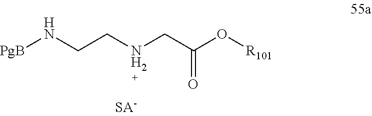
wherein, PgB is a base-labile amine protecting group; R₁₀₁ is a branched or straight chain C₁-C₄ alkyl group or a group of formula I; 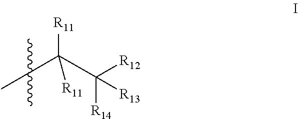
wherein, each R₁₁ is independently H, D, F, C₁-C₆ alkyl, C₃-C₆ cycloalkyl or aryl;
each of R₁₂, R₁₃ and R₁₄ is independently H, D, F, Cl, Br or I, provided however that at least one of R₁₂, R₁₃ and R₁₄ is selected from Cl, Br and I; and
SA⁻ is a sulfonate anion.

66. The method of claim 64, wherein PgB is Fmoc.

67. The method of claim 64, wherein PgB is selected from the group consisting of: Nsc, Bsmoc, Nsmoc, ivDde, Fmoc*, Fmoc(2F), mio-Fmoc, dio-Fmoc, TCP, Pms, Esc, Sps and Cyoc.

68. The method of claim 64, wherein SA⁻ is the sulfonate anion selected from the group consisting of: benzenesulfonate, naphthalenesulfonate, p-toluenesulfonate, p-xylene-2-sulfonate, 2,4,5-trichlorobenzenesulfonate, 2,6-dimethylbenzenesulfonate, 2-mesitylenesulfonate, 2-mesitylenesulfonate dihydrate, 2-methylbenzene sulfonate, 2-ethylbenzenesulfonate, 2-isopropylbenzenesulfonate, 2,3-dimethylbenzenesulfonate, 2,4,6-trimethylbenzenesulfonate and 2,4,6-triisopropyl benzenesulfonate.

69. The method of claim 64, wherein SA⁻ is p-toluenesulfonate.

70. The method of claim 64, wherein the anion Y⁻, is selected from the group consisting of: I⁻, Br⁻, Cl⁻, AcO⁻ (acetate), CF₃COO⁻ (trifluoroacetate), citrate or tosylate.

71. The method of claim 64, further comprising: contacting the compound of formula 54a with at least one equivalent of an acid to thereby produce a compound of formula 55b: 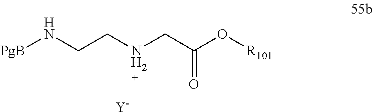
wherein, PgB is a base-labile amine protecting group; R₁₀₁ is a branched or straight chain C₁-C₄ alkyl group or a group of formula I; 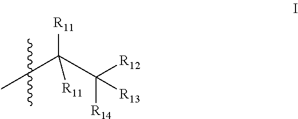
wherein,
each R₁₁ is independently H, D, F, C₁-C₆ alkyl, C₃-C₆ cycloalkyl or aryl;
each of R₁₂, R₁₃ and R₁₄ is independently H, D, F, Cl, Br or I, provided however that at least one of R₁₂, R₁₃ and R₁₄ is selected from Cl, Br and I; and
Y⁻ is an anion.

72. The method of claim 64, wherein the anion, Y⁻, is selected from the group consisting of: I⁻, Br⁻, Cl⁻, AcO⁻ (acetate), CF₃COO⁻ (trifluoroacetate), citrate and tosylate.

73. A method of preparing a PNA monomer ester of formula (II): 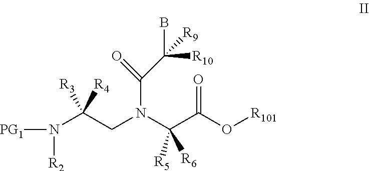
or a pharmaceutically acceptable salt thereof, wherein, B is a nucleobase, optionally comprising one or more protecting groups;
Pg₁ is an amine protecting group;
R₁₀₁ is a group of formula I; 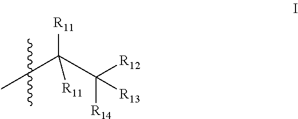
wherein,
each R₁₁ is independently H, D, F, C₁-C₆ alkyl, C₃-C₆ cycloalkyl or aryl;
each of R₁₂, R₁₃ and R₁₄ is independently H, D, F, Cl, Br or I, provided however that at least one of R₁₂, R₁₃ and R₁₄ is selected from Cl, Br and I;
R₂ is H, D or C₁-C₄ alkyl;
each of R₃, R₄, R₅, and R₆ is independently selected from the group consisting of:
H, D, F, and a side chain selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIy, IIIw, IIIx, IIIy, IIIz, IIIaa and IIIab, wherein each of IIIi, IIIj, IIIk, IIIm, IIIn, IIIo, IIIp, IIIq, IIIr, IIIs, IIIt, IIIu, IIIy, IIIw, IIIx, IIIy, and IIIz optionally comprise a protecting group; 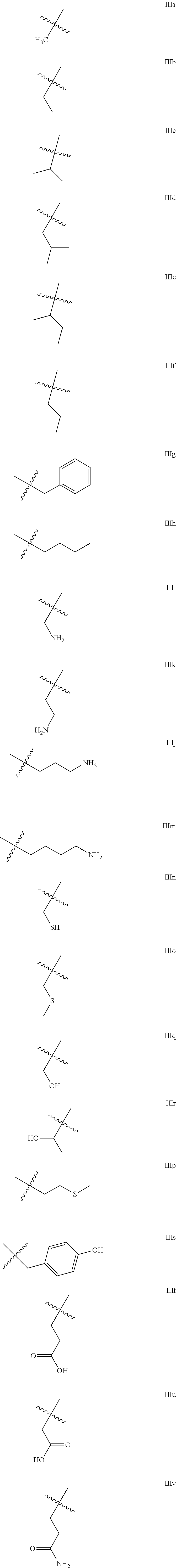 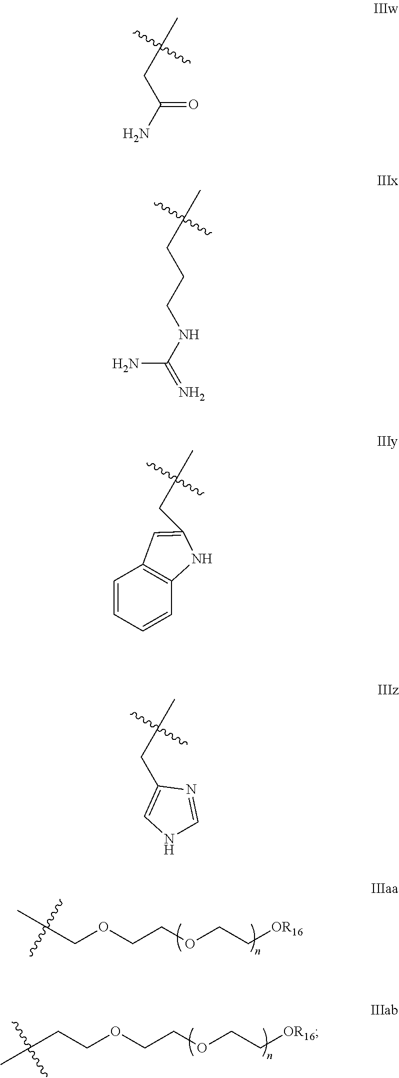
wherein, each of R₉ and R₁₀ is independently selected from the group consisting of: H, D and F;
R₁₆ is selected from H, D and C₁-C₄ alkyl group; and
n is a whole number from 0 to 10, inclusive,
comprising:
a) providing a compound of formula VI: 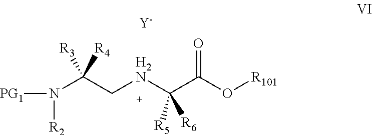
wherein each of Pg₁, R₁₀₁, R₂, R₃, R₄, R₅, and R₆ are as defined, and Y⁻ is an anion (e.g., a sulfonate anion);
b) contacting the compound of formula VI with a nucleobase acid (e.g., a nucleobase acetic acid) of formula IX: 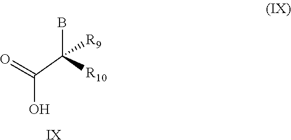
wherein each of R₉, R₁₀, and B are as defined;
in the presence of a carboxylic acid activation agent and a base to form a PNA Monomer Ester of formula (II).

74. The method of claim 73, wherein Pg1 is Fmoc, R₂ is H or methyl, each of R₉ and R₁₀ is H, each R₁₁ is independently H or D, and Y⁻ is selected from benzenesulfonate, p-toluenesulfonate, naphthalenesulfonate, p-xylene-2-sulfonate, 2,4,5-trichlorobenzenesulfonate, 2,6-dimethylbenzenesulfonate, 2-mesitylenesulfonate, 2-mesitylenesulfonate dihydrate, 2-methylbenzene sulfonate, 2-ethylbenzenesulfonate, 2-isopropylbenzenesulfonate, 2,3-dimethylbenzenesulfonate, and 2,4,6-triisopropylbenzenesulfonate.

75. The method of claim 73, wherein Y⁻ is p-toluenesulfonate.

76. The method of claim 73, wherein R₁₂, R₁₃ and R₁₄ are selected from the group consisting of: (i) each of R₁₂, R₁₃ and R₁₄ are Cl; (ii) each of R₁₂, R₁₃ and R₁₄ are Br; (iii) two of R₁₂, R₁₃ and R₁₄ are H and the other of R₁₂, R₁₃ and R₁₄ is Br; and (iv) two of R₁₂, R₁₃ and R₁₄ are H and the other of R₁₂, R₁₃ and R₁₄ is I.

77. The method of claim 73, wherein the nucleobase, B, is independently selected from the group consisting of: adenine, guanine, thymine, cytosine, uracil, pseudoisocytosine, 2-thiopseudoisocytosine, 5-methylcytosine, 5-hydroxymethyl cytosine, xanthine, hypoxanthine, 2-aminoadenine (a.k.a. 2,6-diaminopurine), 2-thiouracil, 2-thiothymine, 2-thiocytosine, 5-chlorouracil, 5-bromouracil, 5-iodouracil, 5-chlorocytosine, 5-bromocytosine, 5-iodocytosine, 5-propynyl uracil, 5-propynyl cytosine, 6-azo uracil, 6-azo cytosine, 6-azo thymine, 7-methylguanine, 7-methyladenine, 8-azaguanine, 8-azaadenine, 7-deazaguanine, 7-deazaadenine, 3-deazaguanine, 3-deazaadenine, 7-deaza-8-aza guanine, 7-deaza-8-aza adenine, 5-propynyl uracil and 2-thio-5-propynyl uracil, including tautomeric forms of any of the foregoing.

78. The method of claim 73 wherein the carboxylic acid activating agent is selected from the group consisting of TMAC, DCC, EDC, HBTU, and HATU.

79. The method of claim 73, wherein the organic base is selected from the group consisting of TEA, NMM, or DIPEA.

80. A method of evaluating a preparation of a compound of formula VI: 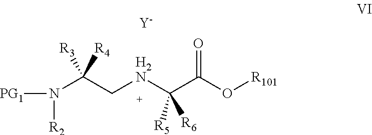
wherein each of Pg₁, R₁₀₁, R₂, R₃, R₄, R₅, R₆, and Y⁻ are as defined in claim 1; comprising:
a) acquiring, e.g., directly or indirectly, a value for the level of an impurity (e.g., a sulfonic acid), e.g., by LCMS or GCMS;
b) evaluating the level of the impurity (e.g., the sulfonic acid), e.g., by comparing the value of the level of the impurity (e.g., the sulfonic acid) with a reference value;
thereby evaluating the preparation.

81. The method of claim 80, wherein the impurity is a sulfonic acid.

82. The method of claim 81, wherein the sulfonic acid is selected from the group consisting of: p-toluenesulfonic acid, benzenesulfonic acid, naphthalenesulfonic acid, p-xylene-2-sulfonic acid, 2,4,5-trichlorobenzenesulfonic acid, 2,6-dimethylbenzenesulfonic acid, 2-mesitylenesulfonic acid, 2-mesitylenesulfonic acid dihydrate, 2-methylbenzene sulfonic acid, 2-ethylbenzenesulfonic acid, 2-isopropylbenzenesulfonic acid, 2,3-dimethylbenzenesulfonic acid, 2,4,6-trimethylbenzenesulfonic acid and 2,4,6-triisopropylbenzenesulfonic acid.

83. A method of evaluating a preparation of a compound of formula VI: 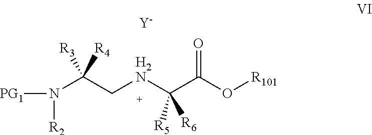
wherein each of Pg₁, R₁₀₁, R₂, R₃, R₄, R₅, R₆, and Y⁻ are as defined in claim 1; comprising:
a) acquiring, e.g., directly or indirectly, a value for the level of an impurity (e.g., a sulfonic acid), e.g., by LCMS;
b) evaluating the level of the impurity (e.g., the sulfonic acid), e.g., by comparing the value of the level of the impurity (e.g., the sulfonic acid) with a reference value;
thereby evaluating the preparation.

84. The method of claim 83, wherein the impurity comprises a sulfonic acid.

85. The method of claim 84, wherein the sulfonic acid is selected from the group consisting of: p-toluenesulfonic acid, benzenesulfonic acid, naphthalenesulfonic acid, p-xylene-2-sulfonic acid, 2,4,5-trichlorobenzenesulfonic acid, 2,6-dimethylbenzenesulfonic acid, 2-mesitylenesulfonic acid, 2-mesitylenesulfonic acid dihydrate, 2-methylbenzene sulfonic acid, 2-ethylbenzenesulfonic acid, 2-isopropylbenzenesulfonic acid, 2,3-dimethylbenzenesulfonic acid, 2,4,6-trimethylbenzenesulfonic acid and 2,4,6-triisopropylbenzenesulfonic acid.
